(19) United States
(12) Patent Application Publication
(10) Pub. No.: US 2025/0335346 A1
(43) Pub. Date: Oct. 30, 2025

(54) CODECS FOR DNA DATA STORAGE

(71) Applicant: Twist Bioscience Corporation, South San Francisco, CA (US)

(72) Inventors: Dominique TOPPANI, San Francisco, CA (US); Stefan PITSCH, Stein am Rhein (CH)

(73) Assignee: Twist Bioscience Corporation, South San Francisco, CA (US)

(21) Appl. No.: 18/858,085

(22) PCT Filed: Apr. 20, 2023
(86) PCT No.: PCT/US2023/019283

§ 371 Date: Oct. 18, 2024

Related U.S. Application Data
(60) Provisional application No. 63/333,305, filed on Apr. 21, 2022.
(60) Provisional application No. 63/338,760, filed on May 5, 2022.
(60) Provisional application No. 63/481,873, filed on Jan. 27, 2023.

(51) Int. Cl. G06F 12/02

(52) U.S. Cl. CPC G06F 12/0223; G06F 2212/20

(57) ABSTRACT

Described herein are systems and methods for encoding digital data into oligonucleotides and decoding the oligonucleotides back into digital data. The encoding and decoding schemes include an inner codec for transforming the digital data into bases, and vice versa. The encoding and decoding schemes also include an outer codec comprising an error correction scheme.

### CROSS-REFERENCE

This application claims the benefit of U.S. Provisional Application No. 63/333,305 filed Apr. 21, 2022, U.S. Provisional Application No. 63/338,760 filed May 5, 2022, and U.S. Provisional Application No. 63/481,873 filed Jan. 27, 2023, which are incorporated by reference in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

DNA is a compelling data storage medium given its superior density, stability, energy-efficiency, and longevity compared to currently used electronic media. However, errors and ambiguities can be introduced or otherwise occur at or during various stages of sequencing and sequencing-related operations and processes. Therefore, there is a need to develop methods to efficiently encode and decode DNA in the presence of such errors.

### SUMMARY

Provided herein are designs and implementations of various codecs that encode digital data (e.g., binary data) into oligo pools and decode pools back into digital data. The codecs may comprise an inner codec for transforming the digital data into bases. The codecs may also comprise an outer code for spreading the data to be stored over many oligos and build redundancy to correct for erasures. The codecs described herein may sustain loss of oligos, and high deletion, mutation and insertion rates during synthesis, storage and/or sequencing. In some embodiments, the codecs described herein are designed for low sequencing coverage. In some embodiments, the codecs described herein are designed for optimizing synthesis of a plurality of polynucleotides.

Further provided herein are methods to retrieve the digital information from the plurality of polynucleotides. The codecs may comprise a bucket-like storage system supporting storage of one or more objects comprising digital information in one or more pool. The codecs may further comprise storage strategies, such as indexing (e.g., index pools) and hashing (e.g., a hashing module) for efficient data storage. The codecs may also build redundancy in the one or more pools to correct for erasures or errors that can occur during storage or retrieval of the digital information.

In one aspect, provided herein are methods for encoding data in a plurality of polynucleotide sequences, comprising: (a) splitting data into a plurality of frames, wherein each frame in the plurality of frames comprises a frame index; (b) applying an outer codec to each frame in the plurality of frames, wherein the outer codec comprises an error correction scheme; (c) dividing each frame into a plurality of lanes, wherein each lane in the plurality of lanes comprises a lane index; (d) shuffling each lane based at least in part on the lane index; and (e) applying an inner codec to encode each lane in a polynucleotide sequence of the plurality of polynucleotide sequences. In some instances, the data comprises a plurality of symbols. In some instances, the data comprises binary data. In some instances, the binary data comprises a byte stream or a byte array. In some instances, the shuffling in (d) comprises a rotation scheme within each lane. In some instances, the shuffling in (d) comprises a pseudorandom process within each lane. In some instances, the shuffling in (d) provides resistance against errors. In some instances, the errors are nucleotide synthesis errors or sequencing errors. In some instances, the errors comprise a deletion, an insertion, or a substitution. In some instances, the error correction scheme comprises a Reed-Solomon (RS) code, a low-density parity-check (LDPC) code, a Turbo-code, a polar code, or any combination thereof. In some instances, the data comprises at least about 1 GB to about 1 TB. In some instances, the plurality of frames comprises about 100 to about 10,000 frames. In some instances, each frame comprises up to about 5000 lanes. In some instances, each lane comprises about 100 to about 300 bits. In some instances, the frame index comprises about 16 bits to about 20 bits. In some instances, the lane index comprises about 12 bits or about 16 bits. In some instances, the polynucleotide sequence is about 100 to about 300 bases in length. In some instances, the frame index and/or the lane index are prepended to each lane prior to (d). In some instances, the applying the inner codec comprises adding redundancy across the plurality of polynucleotide sequences. In some instances, the redundancy is about 5% to about 10%. In some instances, the plurality of polynucleotide sequences can be decoded in the presence of an error in part due to the redundancy across the plurality of polynucleotide sequences. In some instances, the error comprises an insertion, deletion, substitution, or any combination thereof. In some instances, applying the inner codec comprises: (a) combining symbols from a lane, a symbol history, and a symbol position; and (b) generating a base candidate using a lookup table, a hash, or both. In some instances, the methods further comprise performing a base repetition check. In some instances, the symbols are bits. In some instances, the methods further comprise updating the symbol history, incrementing the lane index, incrementing the frame index, or any combination thereof. In some instances, the updated symbol history, incremented lane index, incremented frame index, or any combination thereof is combined with symbols of a subsequent lane. In some instances, the methods further comprise performing GC filtering prior to synthesizing the plurality of the polynucleotide sequences. In some instances, the GC filtering comprises removing about 5% to about 10% of lanes in the plurality of lanes. In some instances, the plurality of polynucleotide sequences comprises about 45% to about 55% GC content. In some instances, at least 90% of the plurality of polynucleotide sequences comprises about 45% to about 55% GC content. In some instances, the applying the inner codec comprises: (a) generating a base candidate for each symbol within a lane using a lookup table; and (b) selecting a next lookup table based at least in part on the previously encoded symbol. In some instances, applying the inner codec comprises applying an encoding scheme.

In another aspect, provided herein are methods for decoding a plurality of polynucleotide sequences to generate an output comprising data, comprising: (a) determining the plurality of polynucleotide sequences; (b) applying an inner codec to the plurality of polynucleotide sequences, wherein the inner codec converts each of the plurality of polynucleotide sequences into a lane comprising a plurality of symbols, wherein the inner codec comprises a mixed decoding algorithm comprising a greedy algorithm and a maximum likelihood (ML) algorithm; (c) arranging lanes of data into frames based on a lane index and a frame index of each lane; and (d) applying an outer codec to the frames, wherein the outer codec comprises an error correction scheme, wherein the frames from the outer codec are merged to generate an output comprising the data. In some instances, the data comprises a plurality of symbols. In some instances, the data comprises binary data. In some instances, the binary data comprises a byte stream or a byte array. In some instances, the inner codec comprises a decoding scheme. In some instances, the method further comprises clustering the polynucleotide sequences prior to (b). In some instances, the clustering is based on an index. In some instances, clustering comprises partially decoding the frame index, the lane index, or both. In some instances, the clustering is performed using a hash function. In some instances, the method further comprises aligning the polynucleotide sequences prior to (b). In some instances, aligning comprises analyzing consensus of the nucleotides using an alignment algorithm. In some instances, the alignment algorithm comprises a pairwise alignment algorithm, a multi-sequence alignment algorithm, or a combination thereof. In some instances, the alignment algorithm comprises: (a) initializing a position for each read in a plurality of reads, wherein initializing comprises aligning a polynucleotide sequence to a position 0; (b) analyzing a consensus of a next one or more bases between each read; (c) determining for each read a decision comprising whether each of the next one or more bases is correct or has an error; (d) incrementing the position given the decision for each read; and (e) repeating steps (b)-(d). In some instances, the error is a deletion, substitution, or an insertion. In some instances, the plurality of reads comprises about 3 to about 10 reads. In some instances, each read is about 100 to about 300 bases in length. In some instances, the next one or more bases is about 2, 3, 4, or 5 bases. In some instances, the mixed decoding algorithm comprises decoding based on transition probabilities from one or more states. In some instances, the one or more states comprise about 100 to about 1000 most probable states. In some instances, the inner codec further comprises a drift term. In some instances, the drift term comprises an integer. In some instances, the integer is associated with a total number of insertions or deletions in a polynucleotide sequence. In some instances, the integer is calculated by summing a value for one or more insertions or a value for one or more deletions in the total number of insertions, deletions, or both. In some instances, the value for each of the one or more insertions comprises+1 and the value for each of the one or more deletions comprises-1. In some instances, (c) comprises de-shuffling the lanes based on the lane index and grouping the lanes into frames based on the frame index. In some instances, the error correction scheme comprises a Reed-Solomon (RS) code, a low-density parity-check (LDPC) code, a Turbo-code, a polar code, or any combination thereof. In some instances, at least one polynucleotide sequence in the plurality of polynucleotide sequences comprises an error. In some instances, the error comprises an insertion, deletion, substitution, or any combination thereof.

In another aspect, provided herein are apparatuses comprising (a) a memory; and (b) a processing device operatively coupled to the memory, wherein the processing device is configured to: (i) split data into a plurality of frames, wherein each frame in the plurality of frames comprises a frame index; (ii) apply an outer codec to each frame in the plurality of frames, wherein the outer codec comprising an error correction scheme; (iii) divide each frame into a plurality of lanes, wherein each lane in the plurality of lanes comprises a lane index; (iv) shuffle each lane based at least in part on the lane index; and (v) apply an inner codec to encode each lane in a polynucleotide sequence. In some instances, the inner codec adds redundancy so that the digital data can be decoded in the presence of an error in the polynucleotide sequence. In some instances, the inner codec comprises an encoding scheme. In some instances, the data comprises a plurality of symbols. In some instances, the data comprises digital data. In some instances, the apparatus further comprises a synthesizer for generating the polynucleotide sequence. In some instances, the memory, the processing device, or both are part of a computing system. In some instances, the computing system comprises a cloud computing system. In some instances, the cloud computing system comprises a private cloud, a public cloud, a hybrid cloud, a multi-cloud, or any combination thereof. In some instances, the cloud computing system comprises an infrastructure as a service (IaaS), platform as a service (PaaS), software as a service (SaaS), or any combination thereof.

In another aspect, provided herein are apparatuses comprising (a) a memory; (b) a sequencing device configured to determine sequences of a plurality of polynucleotides; and (c) a processing device operatively coupled to the memory and the sequencing device, wherein the processing device is configured to: (i) apply an inner codec to the sequences, wherein the inner codec converts each of the sequences into a lane comprising a plurality of symbols, wherein the inner codec comprises a mixed decoding algorithm comprising a greedy algorithm and at a maximum likelihood (ML) algorithm; (ii) arrange the lanes into frames based on a lane index and a frame index in each lanes; and (iii) apply an outer codec to the frames, wherein the outer codec comprises an error correction scheme, wherein the frames from the outer codec are merged to generate an output comprising the data. In some instances, the inner codec comprises a decoding scheme. In some instances, the data comprises a plurality of symbols. In some instances, the data comprises digital data. In some instances, the memory, the processing device, or both are part of a computing system. In some instances, the computing system comprises a cloud computing system. In some instances, the cloud computing system comprises a private cloud, a public cloud, a hybrid cloud, a multi-cloud, or any combination thereof. In some instances, the cloud computing system comprises an infrastructure as a service (IaaS), platform as a service (PaaS), software as a service (SaaS), or any combination thereof.

In another aspect, provided herein is a method for encoding data in polynucleotide sequences, comprising: (a) generating an inner codec comprising a codebook, wherein the codebook is optimized for one or more constraints; and (b) applying the inner codec to encode the data as a plurality of polynucleotide sequences. In some instances, the data comprises a plurality of symbols. In some instances, the data comprises binary data. In some instances, the one or more constraints are related to nucleic acid synthesis, post-processing, storage, sequencing, or any combination thereof. In some instances, the nucleic acid synthesis comprises electrochemical synthesis, enzymatic synthesis, phosphoramidite synthesis, inkjet printing, or any combination thereof. In some instances, the one or more constraints related to nucleic acid synthesis comprises a synthesis error. In some instances, the synthesis error comprises an insertion, deletion, or mutation. In some instances, post-processing comprises one or more of ligation, cleavage, hybridization, denaturation, fixation to a solid support, extension, error correction, enrichment, isolation, purification, or amplification. In some instances, storage comprises cold data storage. In some instances, storage comprises nucleic acid storage in a liquid phase or solid phase. In some instances, one or more constraints related to storage comprises temperature, humidity, pressure, salinity, pH, concentration, time, light, UV, O₂, or any combination thereof. In some instances, the temperature comprises room temperature. In some instances, sequencing comprises next generation sequencing, parallel sequencing, single-molecule real-time sequencing, nanopore sequencing, sequencing by synthesis, Sanger sequencing, or any combination thereof. In some instances, further comprising (c) synthesizing a plurality of polynucleotides comprising the plurality of polynucleotide sequences. In some instances, the codebook comprises codewords that are generated based in part on a base order. In some instances, the base order comprises predetermined base transitions. In some instances, the inner codec comprises two or more codebooks. In some instances, each of the two or more codebooks encodes a layer during synthesis of the plurality of polynucleotides. In some instances, the layer comprises extension of each polynucleotide of the plurality of polynucleotides by at least one base. In some instances, synthesis of the layer comprises one or more cycles, wherein each of the one or more cycles comprises flowing a base according to the one or more base transitions of the codebook. In some instances, a cycle of the one or more cycles comprises addition of one or more of A, T, C, or G. In some instances, each of the two or more codebooks comprises a different base order. In some instances, the codebook comprises about 12 codewords. In some instances, (b) comprises mapping the data to a plurality of polynucleotide sequences based on the codebook. In some instances, the inner codec is further optimized against one or more constraints comprising a length, GC content, repeats, errors, or any combination thereof of the plurality of polynucleotide sequences. In some instances, 40% to 60% of the plurality of polynucleotide sequences encode for redundancy. In some instances, synthesizing comprises a number of synthesis cycles. In some instances, the number of synthesis cycles is reduced compared to the number of synthesis cycles needed to synthesize polynucleotide sequences not encoded using the inner codec. In some instances, the reduced number of synthesis cycles is based in part on the flow order. In some instances, the number of synthesis cycles is reduced by at least 30%. In some instances, the number of synthesis cycles is reduced by 50%. In some instances, the number of synthesis cycles is less than 300 for a polynucleotide sequence comprising 100 bases. In some instances, the number of synthesis cycles is about 155 for a polynucleotide sequence comprising 100 bases. In some instances, the polynucleotide sequence comprises one or more of A, T, C, or G. In some instances, (c) comprises synthesizing the plurality of polynucleotides on a solid support. In some instances, the solid support comprises a plurality of features. In some instances, greater than 25% of the plurality of features are deblocked per synthesis cycle. In some instances, at least 50% of the plurality of features are deblocked per synthesis cycle. In some instances, each of the plurality of polynucleotide sequences have a same length. In some instances, 80% to 100% of the plurality of polynucleotide sequences have a same length. In some instances, further comprising sequencing the plurality of polynucleotides to generate a plurality of output sequences. In some instances, the plurality of output sequences are decoded using a greedy algorithm, a maximum likelihood (ML) algorithm, or a mixed greedy ML algorithm. In some instances, the plurality of output sequences are decoded based at least in part by calculating a probability of an error. In some instances, the error comprises a deletion, insertion, mutation, or any combination thereof.

In another aspect, provided herein is hybrid organic-in silico platform for encoding data, the platform composing: (a) a computing system comprising at least one processor and instructions executable by the at least one processor to perform operations comprising: (i) generating an inner codec comprising a codebook, wherein the codebook is optimized for one or more constraints; and (ii) applying the inner codec to encode the data as a plurality of polynucleotide sequences; and (b) a synthesizer for generating a plurality of polynucleotides comprising the plurality of polynucleotide sequences. In some instances, the data comprises a plurality of symbols. In some instances, the one or more constraints are related to nucleic acid synthesis, post-processing, storage, sequencing, or any combination thereof. In some instances, the nucleic acid synthesis comprises electrochemical synthesis, enzymatic synthesis, phosphoramidite synthesis, inkjet printing, or any combination thereof. In some instances, the one or more constraints related to nucleic acid synthesis comprises a synthesis error. In some instances, the synthesis error comprises an insertion, deletion, or mutation. In some instances, post-processing comprises one or more of ligation, cleavage, hybridization, denaturation, fixation to a solid support, extension, error correction, enrichment, isolation, purification, and amplification. In some instances, storage comprises cold data storage. In some instances, storage comprises nucleic acid storage in a liquid phase or solid phase. In some instances, one or more constraints related to storage comprises temperature, humidity, pressure, salinity, pH, concentration, time, light, UV, O₂, or any combination thereof. In some instances, the temperature comprises room temperature. In some instances, sequencing comprises next generation sequencing, parallel sequencing, single-molecule real-time sequencing, nanopore sequencing, sequencing by synthesis, Sanger sequencing, or any combination thereof. In some instances, the computing system comprises a cloud computing system. In some instances, the cloud computing system comprises a private cloud, a public cloud, a hybrid cloud, a multi-cloud, or any combination thereof. In some instances, the cloud computing system comprises an infrastructure as a service (IaaS), platform as a service (PaaS), software as a service (Saas), or any combination thereof. In some instances, the codebook comprises codewords that are generated based in part on the base order. In some instances, the base order comprises predetermined base transitions. In some instances, the inner codec comprises two or more codebooks. In some instances, each of the two or more codebooks encodes a layer during synthesis of the plurality of polynucleotides. In some instances, the layer comprises extension of each polynucleotide of the plurality of polynucleotides by at least one base. In some instances, synthesis of the layer comprises one or more cycles, wherein each of the one or more cycles comprises flowing a base according to the one or more base transitions of the codebook. In some instances, a cycle of the one or more cycles comprises addition of one or more of A, T, C, or G. In some instances, each of the two or more codebooks comprises a different base order. In some instances, the instructions further cause the synthesizer to generate the plurality of polynucleotides. In some instances, further comprising a sequencer for sequencing the plurality of polynucleotides to generate a plurality of output sequences. In some instances, the instructions further cause the computing system to receive the plurality of output sequences. In some instances, the computing system further performs operations comprising: (iii) decoding the plurality of output sequences. In some instances, the plurality of output sequences are decoded using a greedy algorithm, a maximum likelihood (ML) algorithm, or a mixed greedy ML algorithm. In some instances, the plurality of output sequences are decoded based at least in part by calculating a probability of a deletion, insertion, mutation, or any combination thereof. In some instances, further comprising a storage unit for storing the plurality of polynucleotides. In some instances, the operations further comprise transferring the plurality of polynucleotides between the synthesizer, the sequencer, the storage unit, or any combination thereof. In some instances, the specific base transitions allow for synthesis according to a flow order. In some instances, the codebook comprises about 12 codewords. In some instances, wherein (a) (ii) comprises mapping the data to a plurality of polynucleotide sequences based on the codebook. In some instances, the inner codec is further optimized against constraints comprising a length, GC content, repeats, or any combination thereof of the plurality of polynucleotide sequences. In some instances, 40% to 60% of the plurality of polynucleotide sequences encode for redundancy. In some instances, generating the plurality of polynucleotides comprises a number of synthesis cycles. In some instances, the number of synthesis cycles is reduced compared to the number of synthesis cycles needed to synthesize polynucleotide sequences not encoded using the inner codec. In some instances, the reduced number of synthesis cycles is based in part on the flow order. In some instances, the number of synthesis cycles is reduced by at least 30%. In some instances, the number of synthesis cycles is reduced by 50%. In some instances, the number of synthesis cycles is less than 300 for a polynucleotide sequence comprising 100 bases. In some instances, the number of synthesis cycles is about 155 for a polynucleotide sequence comprising 100 bases. In some instances, the polynucleotide sequence comprises one or more A, T, C, or G. In some instances, generating the plurality of polynucleotides comprises base-by-base synthesis. In some instances, the synthesizer comprises a solid-support comprising a plurality of features. In some instances, each of the plurality of features are independently addressable through one or more electrodes of the solid-support. In some instances, each of the plurality of features are addressable through masking. In some instances, the masking comprises a physical barrier. In some instances, the masking comprises controlling reactivity at one or more of the plurality of features. In some instances, controlling reactivity comprises deprotection at one or more of the plurality of features. In some instances, the deprotection comprises acid-generation. In some instances, the deprotection electrochemical deprotection. In some instances, greater than 25% of the plurality of features are deblocked per synthesis cycle. In some instances, at least 50% of the plurality of features are deblocked per synthesis cycle. In some instances, each of the plurality of polynucleotide sequences have a same length. In some instances, 80% to 100% of the plurality of polynucleotide sequences have a same length.

In one aspect, provided herein are systems for storing data in DNA comprising: one or more processing units; a memory in communication with the one or more processing units, instructions stored in the memory and executed on the one or more processing units that cause the system to: generate a plurality of pools, wherein each of the plurality of pools comprises a pool descriptor, a pool item comprising a payload of the data, and an end descriptor; determine a first one or more hashes of the payload for each pool item; and apply an encoding scheme to encode the plurality of pools as sequences of a plurality of polynucleotides. In some embodiments, the encoding scheme comprises an inner codec, an outer codec, or both that is described herein. In some embodiments, the data comprises an item of information or digital information described herein. In some embodiments, the data comprises one or more objects. In some embodiments, the one or more processing units, the memory, or both are part of a computing system. In some instances, the computing system comprises a cloud computing system. In some instances, the cloud computing system comprises a private cloud, a public cloud, a hybrid cloud, a multi-cloud, or any combination thereof. In some instances, the cloud computing system comprises an infrastructure as a service (IaaS), platform as a service (PaaS), software as a service (SaaS), or any combination thereof. In some embodiments, instructions stored in the memory and executed on the one or more processing units that cause the system to determine a second one or more hashes of each of the one or more objects. In some embodiments, the one or more objects comprises a file or metadata associated with the file. In some embodiments, the pool descriptor comprises a version, a pool ID, a list of pool item descriptors, or any combination thereof. In some embodiments, the pool ID comprises a unique ID. In some embodiments, the unique ID comprises a universal unique identifier (UUID) or a content ID. In some embodiments, the list of pool item descriptors comprises a path of an object, a size of an object, a range of the pool item within an object, offset of the pool item in a pool, or any combination thereof. In some embodiments, each of the one or more pool items further comprises a hash of the pool item from the first one or more hashes. In some embodiments, the end pool descriptor comprises a list of object descriptors. In some embodiments, the list of object descriptors comprises a path of an object, a hash of an object from the first one or more hashes, or a combination thereof. In some embodiments, each of the plurality of pools is about 1 GB to about 1 TB. In some embodiments, the plurality of pools comprise redundant pools. In some embodiments, the first one or more hashes, the second one or more hashes, or both are determined using a hashing module. In some embodiments, the hashing module is executed on the one or more processing units. In some embodiments, the first one or more hashes require less memory than the one or more objects. In some embodiments, the second one or more hashes require less memory than the one or more pool items. In some embodiments, the hashing module comprises a hash function. In some embodiments, the hash function comprises SHA-224, SHA-256, SHA-384, SHA-512, SHA-512/224, or SHA-512/256. In some embodiments, the instructions further cause the system to generate one or more index pools. In some embodiments, the one or more index pools comprise an index pool descriptor and a list of object indexing. In some embodiments, the index pool descriptor comprises a version, a pool ID, a size of a pool, and a timestamp. In some embodiments, the pool ID comprises a unique ID. In some embodiments, the unique ID comprises a UUID or a content ID. In some embodiments, the list of object indexing comprises a path of an object, a hash of an object, a list of object fragments, a list of object metadata, or any combination thereof. In some embodiments, the list of object fragments comprises a pool ID of a pool containing a fragment, a range of a fragment, or a combination thereof. In some embodiments, the list of object metadata comprises the type of metadata, the metadata payload, or a combination thereof. In some embodiments, the type of metadata comprises, a list of keywords attached to an object, a thumbnail picture, a text summary, an ID range for a sorted key-value database, a timestamp, a version, or any combination thereof. In some embodiments, each of the one or more index pools is about 1 GB to about 1 TB. In some embodiments, the instructions stored in the memory and executed on the one or more processing units that cause the system to retrieve the data stored in the DNA. In some embodiments, the instructions comprise: applying a decoding scheme to decode the sequences of the plurality of polynucleotides in each of the plurality of pools; and verifying at least the payload of each pool item using the first one or more hashes.

In one aspect, provided herein are devices for storing information in DNA comprising: one or more compartments, wherein each compartment comprises: (a) a library comprising a plurality of polynucleotides, wherein the library encodes a pool comprising information corresponding to one or more objects; and (b) a medium for storing the plurality of polynucleotides. In some embodiments, the information comprises an item of information or digital information described herein. In some embodiments, the information comprises a plurality of symbols. In some embodiments, the one or more compartments are in communication. In some embodiments, the one or more compartments are not in communication. In some embodiments, the medium comprises a solid, a liquid, a gas, or any combination thereof. In some embodiments, a medium comprises a salt solution at a molar ratio of less than 20:1 salt cation to phosphate groups in the DNA. In some embodiments, the salt solution is dried to create a dried product. In some embodiments, the device further comprises a solid support comprising a surface. In some embodiments, the device further comprises a plurality of structures located on the surface, wherein the plurality of polynucleotide are extended from the plurality of structures. In some embodiments, the one or more objects comprises a file or metadata associated with the file. In some embodiments, the pool comprises a pool descriptor, one or more pool items, and an end pool descriptor. In some embodiments, the pool descriptor comprises a version, a pool ID, a list of pool item descriptors, or any combination thereof. In some embodiments, the pool ID comprises a unique ID. In some embodiments, the unique ID comprises a universal unique identifier (UUID) or a content ID. In some embodiments, the list of pool item descriptors comprises a path of an object, a size of an object, a range of the pool item within an object, offset of the pool item in a pool, or any combination thereof. In some embodiments, each of the one or more pool items comprises a data payload, a hash of the pool item, or a combination thereof. In some embodiments, the end pool descriptor comprises a list of object descriptors. In some embodiments, the list of object descriptors comprises a path of an object, a hash of an object, or a combination thereof. In some embodiments, the pool comprises about 1 GB to about 1 TB of digital information. In some embodiments, the device further comprises one or more second compartments, wherein each of the one or more second compartments comprises a second library encoding an index pool. In some embodiments, the one or more index pools comprise an index pool descriptor and a list of object indexing. In some embodiments, the index pool descriptor comprises a version, a pool ID, a size of a pool, and a timestamp. In some embodiments, the pool ID comprises a unique ID. In some embodiments, the unique ID comprises a UUID or a content ID. In some embodiments, the list of object indexing comprises a path of an object, a hash of an object, a list of object fragments, a list of object metadata, or any combination thereof. In some embodiments, the list of object fragments comprises a pool ID of a pool containing a fragment, a range of a fragment, or a combination thereof. In some embodiments, the list of object metadata comprises the type of metadata, the metadata payload, or a combination thereof. In some embodiments, the type of metadata comprises, a list of keywords attached to an object, a thumbnail picture, a text summary, an ID range for a sorted key-value database, a timestamp, a version, or any combination thereof. In some embodiments, each of the one or more index pools is about 1 GB to about 1 TB.

In another aspect, provided herein are methods for storing data in a plurality of polynucleotides, comprising: generating a plurality of pools, wherein each of the plurality of pools comprises a pool descriptor, a pool item comprising a payload of the data, and an end descriptor; determining a first one or more hashes of the payload for each pool item; and applying an encoding scheme to encode the plurality of pools as sequences of a plurality of nucleotides. In some embodiments, the encoding scheme comprises an inner codec, an outer codec, or both that is described herein. In some embodiments, the data comprises an item of information or digital information described herein. In some instances, the data comprises a plurality of symbols. In some embodiments, the data comprises one or more objects. In some embodiments, the method further comprises determining a second one or more hashes of each of the one or more objects. In some embodiments, further comprising storing the plurality of polynucleotides. In some embodiments, polynucleotides of the plurality of polynucleotides corresponding to each pool of the plurality of pools are stored in separate containers of a data storage system. In some embodiments, further comprising generating the plurality of polynucleotides. In some embodiments, generating the plurality of polynucleotides comprises phosphoramidite-based synthesis of deoxyribonucleic acid (DNA). In some embodiments, a reagent for the phosphoramidite-based synthesis comprises a nucleoside phosphoramidite, an oxidizer, an activator, or a deblocker or the solvent comprises acetonitrile. In some embodiments, generating the plurality of polynucleotides comprises enzymatic DNA synthesis. In some embodiments, a reagent for enzymatic DNA synthesis comprises terminal deoxynucleotidyl transferase (TdT) or a deblocker or the solvent comprises water. In some embodiments, the one or more objects comprises a file or metadata associated with the file. In some embodiments, the pool descriptor comprises a version, a pool ID, a list of pool item descriptors, or any combination thereof. In some embodiments, the pool ID comprises a unique ID. In some embodiments, the unique ID comprises a universal unique identifier (UUID) or a content ID. In some embodiments, the list of pool item descriptors comprises a path of an object, a size of an object, a range of the pool item within an object, offset of the pool item in a pool, or any combination thereof. In some embodiments, each of the one or more pool items further comprises a hash of the pool item from the first one or more hashes. In some embodiments, the end pool descriptor comprises a list of object descriptors. In some embodiments, the list of object descriptors comprises a path of an object, a hash of an object from the first one or more hashes, or a combination thereof. In some embodiments, each of the plurality of pools is about 1 GB to about 1 TB. In some embodiments, the plurality of pools comprise redundant pools. In some embodiments, the first one or more hashes, the second one or more hashes, or both are determined using a hashing module. In some embodiments, the second one or more hashes require less memory than the one or more objects. In some embodiments, the first one or more hashes require less memory than the one or more pool items. In some embodiments, the hashing module comprises a hash function. In some embodiments, the hash function comprises SHA-224, SHA-256, SHA-384, SHA-512, SHA-512/224, or SHA-512/256. In some embodiments, further comprising creating one or more index pools. In some embodiments, the one or more index pools comprise an index pool descriptor and a list of object indexing. In some embodiments, the index pool descriptor comprises a version, a pool ID, a size of a pool, and a timestamp. In some embodiments, the pool ID comprises a unique ID. In some embodiments, the unique ID comprises a UUID or a content ID. In some embodiments, the list of object indexing comprises a path of an object, a hash of an object, a list of object fragments, a list of object metadata, or any combination thereof. In some embodiments, the list of object fragments comprises a pool ID of a pool containing a fragment, a range of a fragment, or a combination thereof. In some embodiments, the list of object metadata comprises the type of metadata, the metadata payload, or a combination thereof. In some embodiments, the type of metadata comprises, a list of keywords attached to an object, a thumbnail picture, a text summary, an ID range for a sorted key-value database, a timestamp, a version, or any combination thereof. In some embodiments, each of the one or more of index pools is about 1 GB to about 1 TB.

In another aspect, provided herein are methods for retrieving data stored in a plurality of polynucleotides, comprising: determining sequences of the plurality of polynucleotides, wherein the plurality of polynucleotides are in a plurality of pools; applying a decoding scheme to decode the sequences of the plurality of polynucleotides in each of the plurality of pools, wherein each pool comprises a pool descriptor, a pool item comprising a payload of the data, and end descriptor; and verifying at least the payload of each pool item using a first one or more hashes. In some embodiments, the decoding scheme comprises an inner codec, an outer codec, or both that is described herein. In some embodiments, the data comprises an item of information or digital information described herein. In some embodiments, the data comprises one or more objects. In some embodiments, the one or more objects comprises a file or metadata associated with the file. In some embodiments, the method further comprises verifying the one or more objects using a second one or more hashes. In some embodiments, verifying at least the payload comprises verifying the first one or more hashes using a hash function. In some embodiments, the method further comprises combining the payload from each pool item to retrieve the data. In some embodiments, method further comprises storing the data on a memory. In some embodiments, each of the plurality of pools is about 1 GB to about 1 TB. In some embodiments, verifying the one or more objects comprises verifying the second one or more hashes using a hash function. In some embodiments, the hash function comprises SHA-224, SHA-256, SHA-384, SHA-512, SHA-512/224, or SHA-512/256. In some embodiments, determining the sequences comprises sequencing the plurality of polynucleotides. In some embodiments, sequencing comprises next generation sequencing, parallel sequencing, single-molecule real-time sequencing, nanopore sequencing, sequencing by synthesis, Sanger sequencing, or any combination thereof. In some embodiments, the method further comprises accessing an index pool of one or more index pools to determine a plurality of pools comprising the one or more objects. In some embodiments, the index pool comprise an index pool descriptor and a list of object indexing. In some embodiments, the index pool descriptor comprises a version, a pool ID, a size of a pool, and a timestamp. In some embodiments, the pool ID comprises a unique ID. In some embodiments, the unique ID comprises a UUID or a content ID. In some embodiments, the list of object indexing comprises a path of an object, a hash of an object, a list of object fragments, a list of object metadata, or any combination thereof. In some embodiments, the list of object fragments comprises a pool ID of a pool containing a fragment, a range of a fragment, or a combination thereof. In some embodiments, the list of object metadata comprises the type of metadata, the metadata payload, or a combination thereof. In some embodiments, the type of metadata comprises, a list of keywords attached to an object, a thumbnail picture, a text summary, an ID range for a sorted key-value database, a timestamp, a version, or any combination thereof. In some embodiments, each of the one or more of index pools is about 1 GB to about 1 TB.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of an encoding scheme for a low level codec according to some embodiments.

FIG. 2 shows a non-limiting example of a decoding scheme for a low level codec according to some embodiments.

FIG. 3 shows a non-limiting example of an encoding scheme including an outer codec, according to some embodiments.

FIG. 4 shows a non-limiting example of an encoding scheme including shuffling lanes of data, according to some embodiments.

FIG. 5 shows a shows a non-limiting example of an encoding scheme including a first inner codec, according to some embodiments.

FIG. 6 shows a non-limiting example of an encoding scheme including a second inner codec, according to some embodiments.

FIG. 7 shows a non-limiting example of a decoding scheme including an inner codec and an outer codec, according to some embodiments.

FIG. 8 shows a non-limiting example of a greedy algorithm for decoding according to some embodiments.

FIG. 9 shows a non-limiting example of a maximum likelihood (ML) algorithm for decoding according to some embodiments.

FIG. 10 shows a non-limiting example of a computing device; in this case, a device with one or more processors, memory, storage, and a network interface.

FIG. 11A shows a non-limiting example of a “lift-off” process for fabrication of a polynucleotide synthesis surface according to some embodiments.

FIG. 11B shows a non-limiting example of a wet etch process for fabrication of a polynucleotide synthesis surface according to some embodiments. The process may also be adapted to a dry etch process.

FIG. 12 shows a non-limiting example of an encoding scheme for a high level codec according to some embodiments.

FIG. 13 shows a non-limiting example of a decoding scheme for a high level codec according to some embodiments.

FIG. 14 shows a non-limiting example of digital information storage according to some embodiments.

FIG. 15 shows a non-limiting example of generating a hash according to some embodiments.

FIG. 16 shows a non-limiting example of system for synthesizing, storing, and sequencing a plurality of polynucleotides according to some embodiments.

FIGS. 17A-17G show non-limiting examples of a structure or a compartment for storing a plurality of polynucleotides according to some embodiments. FIG. 17A shows a structure that is substantially tubular. FIG. 17B shows a structure comprising a cap and a body that are flush-welded together. FIG. 17C shows structure comprising a removable screw-cap. FIG. 17D shows a structure comprising a septum. FIG. 17E shows a structure comprising two rounded, pill-shaped halves that form a seal when one half is inserted into the other. FIG. 17F shows a structure comprising a substantially flat, disc container with sealable lid. FIG. 17G shows a structure comprises a box with an optionally attached lid.

### DETAILED DESCRIPTION

Provided herein are methods and systems for storing digital information in nucleic acids. As in many storage mediums, synthetic DNA can have inherent errors such as deletions, insertions, mutations, or fragmentations, which can lead to erasure of complete oligonucleotides. There may also be loss of some oligonucleotides due to aging or sample processing. Typical techniques used in computer science and telecommunication only address erasure and/or mutations, but do not address specific behavior of oligonucleotide pools. For example, sequencing oligonucleotide pools provide oligos in random order, whereas typical storage mediums like hard drives provide a stream of data in a known and expected order that is created during writing. Moreover, many codecs for storing digital information focus on encoding digital information in nucleic acids, but may not provide a way to store and retrieve a structured list of files. As such, provided herein are codecs and implementations that can take a number of “objects” and efficiently store them as or retrieve them from one or more pools. An object may comprise a file or metadata associated with the file. Such codec implementation can be combined with a low level codec for encoding digital information in nucleic acids and/or outer codecs, for example comprising error correction codes, such as, but not limited to, those described herein.

In some instances, the methods encode data in a plurality of polynucleotide sequences. The data may be represented as a plurality of symbols. In some instances, methods comprise one or more step of: splitting data into a plurality of frames; applying an outer codec to each frame in the plurality of frames; dividing each frame into a plurality of lanes; shuffling each lane based at least in part on the lane index; and applying an inner codec (e.g., encoding scheme) to encode each lane in a polynucleotide sequence of the plurality of polynucleotide sequences. In some instances, each frame in the plurality of frames comprises a frame index. In some instances, the outer codec comprises an error correction scheme. In some instances, each lane in the plurality of lanes comprises a lane index.

In some instances, methods decode a plurality of polynucleotide sequences to generate an output comprising data. The data may be represented as a plurality of symbols. In some instances, methods comprise one or more step of: determining the plurality of polynucleotide sequences; applying an inner codec (e.g., decoding scheme) to the plurality of polynucleotide sequences; arranging the lanes of data into frames based on a lane index and a frame index in each of the lanes of data; and applying an outer codec to the frames. In some instances, the inner codec converts each of the plurality of polynucleotide sequences into a lane comprising a plurality of symbols. In some instances, the inner codec comprises a mixed decoding algorithm comprising a greedy algorithm and a maximum likelihood (ML) algorithm. In some instances, the outer codec comprises an error correction scheme. In some instances, the frames from the outer codec are merged to generate an output comprising the data.

In some instances, the systems encode data in a plurality polynucleotide sequences. In some instances, systems comprise an apparatus comprising one or more of: a memory; and a processing device operatively coupled to the memory. In some instances, the processing device is configured to perform one or more of the steps comprising: split the data into a plurality of frames; apply an outer codec to each frame in the plurality of frames; divide each frame into a plurality of lanes; shuffle each lane based at least in part on the lane index; and apply an inner codec to encode each lane in a polynucleotide sequence. In some instances, each frame in the plurality of frames comprises a frame index. In some instances, each lane in the plurality of lanes comprises a lane index. In some instances, the outer codec comprising an error correction scheme. In some instances, the inner codec adds redundancy so that the data can be decoded in the presence of an error in the polynucleotide sequence. In some instances, the inner codec comprises an encoding scheme.

In some instances, the systems decode a plurality of polynucleotide sequences to generate an output comprising data. In some instances, systems comprise an apparatus comprising one or more of: a memory; a sequencing device configured to determine the plurality of polynucleotide sequences; and a processing device operatively coupled to the memory. In some instances, the processing device is configured to perform one or more of the steps comprising: apply an inner codec to the plurality of polynucleotide sequences; arrange the lanes of data into frames based on a lane index and a frame index in each of the lanes of data; and apply an outer codec to the frames. In some instances, inner codec converts each of the sequences into a lane comprising a plurality of symbols. In some instances, the inner codec comprises a mixed decoding algorithm comprising a greedy algorithm and a maximum likelihood (ML) algorithm. In some instances, the outer codec comprises an error correction scheme. In some instances, the frames from the outer codec are merged to generate an output comprising the data. In some instances, the inner codec comprises a decoding scheme.

In some instances, methods encode data in polynucleotide sequences. In some instances, methods comprise one or more steps of: (a) generating an inner codec comprising a codebook, wherein the codebook is optimized for one or more constraints; and (b) applying the inner codec to encode the data as a plurality of polynucleotide sequences. In some instances, the method further comprises generating the plurality of polynucleotides comprising the plurality of polynucleotide sequences.

In some instances, provided herein are hybrid organic-in silico platforms for encoding data. The platform comprising one or more of: a computing system comprising at least one processor and instructions executable by the at least one processor to perform operations; and a synthesizer for generating a plurality of polynucleotides comprising the plurality of polynucleotide sequences. In some instances, the operations comprise one or more of: generating an inner codec comprising a codebook, wherein the codebook is optimized for one or more constraints; and applying the inner codec to encode the data as a plurality of polynucleotide sequences.

In some instances, the systems store information in DNA. In some instances, the system comprises any one of or a combination of: one or more processing units; a memory in communication with the one or more processing units, and instructions stored in the memory and executed on the one or more processing units. In some instances, the instructions cause the system to do any one of or a combination of: split digital information of one or more objects into a plurality of pools; generate a pool descriptor, one or more pool items, and an end pool descriptor in each of the plurality of pools; determine a first one or more hashes of a data payload of each of the one or more pool items and a second one or more hashes of each of the one or more objects; and apply an encoding scheme to encode the digital information in the plurality of pools as a plurality of polynucleotides.

In some instances, the devices for storing information in DNA. In some instances, the device comprises one or more compartments. In some instances, each compartment comprises any one of or a combination of: a library comprising a plurality of polynucleotides; and a medium for storing the plurality of polynucleotides. In some instances, the library encodes a pool comprising the information corresponding to one or more objects.

In some instances, the methods store data in a plurality of polynucleotides. In some instances, the method comprises any one of or a combination of: generating a plurality of pools; determining a first one or more hashes of the payload for each pool item; and applying an encoding scheme to encode the plurality of pools as sequences of a plurality of nucleotides. In some instances, each of the plurality of pools comprises a pool descriptor, a pool item comprising a payload of the data, and an end descriptor.

In some instances, the methods retrieve data stored in a plurality of polynucleotides. In some instances, the method comprises any one of or a combination of: determining sequences of the plurality of polynucleotides; applying a decoding scheme to decode the sequences of the plurality of polynucleotides in each of the plurality of pools; and verifying at least the payload of each pool item using a first one or more hashes. In some instances, the plurality of polynucleotides are in a plurality of pools. In some instances, each pool comprises a pool descriptor, a pool item comprising a payload of the data, and end descriptor.

Further provided herein are methods and systems for optimizing synthesis of polynucleotides. In some instances, synthesis is optimized using a synthesis optimized codec, such as those provided herein. The polynucleotides may be synthesized according to a device provided herein. Electronic synthesis typically comprises deblocking specific sites (e.g., features or loci on a surface for polynucleotide synthesis) and flowing a specific base (e.g., nucleic acid monomer), which are repeated for each base. This implies that polynucleotides without specific base ordering can require 4 cycles per layer (e.g., A, T, C, G), especially when synthesizing millions of polynucleotides together as the chance of sections of polynucleotides matching in synthesis order is very low. For example, a surface is masked to protect specific sites (wherein each site comprises a unique polynucleotide, and is independently addressable) from base addition, a base is coupled to unprotected sites, and then the mask is changed to allow for coupling bases at different sites. A layer generally comprises an extension of each polynucleotide by at least one base. For example, if the polynucleotides are M bases long, then synthesis can require 4×M cycles assuming 4 cycles per addition of a single nucleic acid to a polynucleotide. This approach can be more costly as it can take more time, more reagents, or both. It can also increase chances of DNA damage as each cycle requires an oxidation step and deblocking step, which can result in higher error rates.

Methods, systems, and platforms to optimize synthesis can comprise an inner codec optimized to generate polynucleotides following a specific order of base synthesis. This can allow synthesis of polynucleotides with less than 4×M cycles, where M is the number of bases of a polynucleotide. This approach can also provide redundancy for error correction, such as using an outer codec or error correction code (ECC). This approach may also accelerate synthesis of polynucleotides relative to a synthesis approach that is not optimized (e.g., requires 4×M cycles), when the polynucleotides being synthesized encode the same amount of data. In some instances, a mixtures of bases (e.g., two or three) are flowed across the surface in a single cycle. In some instances, the synthesis method is configured for use with one or more codebooks provided herein. An unoptimized synthesis approach as described herein may generally refer to synthesis of polynucleotides without base ordering. In some instances, the synthesis rate is accelerated about 1.5 times, 2 times, 2.5 times, 3 times, 3.5 times, or 4 times relative to an unoptimized synthesis approach. In some instances, the synthesis rate is accelerated up to 2 times, 2.5 times, 3 times, 3.5 times, or 4 times relative to an unoptimized synthesis approach. In some instances, the synthesis rate is accelerated at most about 1.5 times, 2 times, 2.5 times, 3 times, or 3.5 times relative to an unoptimized synthesis approach. In some instances, the synthesis rate is accelerated while improving DNA quality, as less oxidation steps are required. In some instances, the synthesis rate is accelerated while reducing errors.

In some instances, the methods provided herein encode data. The data may be digital information or an item of information. The data may be represented as one or more symbols. In some examples, the one or more symbols comprise numerical values, such as binary data. In some instances, the data represented as a set of symbols is encoded as a different set of symbols using a codec. In some instances, such codec is referred to as an inner codec. In some instances, the different set of symbols comprises a sequence of symbols, such as a polynucleotide sequence.

Methods described herein may comprise use or generation of inner codecs. In some instances, the method comprises generating an inner codec comprising a codebook. In some instances, a codebook comprises the contents, structure, and layout of a data collection (e.g., digital information encoded in nucleic acids). In some instances, the inner codec comprises two or more codebooks. In some instances, each of the two or more codebooks encodes a layer during synthesis of the polynucleotides. In some instances, the codebook is optimized for one or more constraints. In some instances, the one or more constraints are related to nucleic acid synthesis, post-processing, storage, sequencing, or any combination thereof.

In some instances, the codebook is generated with a base order. In some instances, the codebook is optimized to for one or more base transitions. In some instances, the base order generates the one or more base transitions. Such one or more base transitions may be referred to as specific base transitions or predetermined base transitions. In some instances, each of the two or more codebooks comprises a different base order. In some instances, each of the two or more codebooks comprises a different one or more base transitions. In some instances, the codebook is optimized for specific base transitions at a given layer, cycle index, history, or any combination thereof. In some examples, the history comprises one or more of the previous layers, the one or more codebooks encoding the previous one or more layers, the cycle indices of the one or more previous layers, or any combination thereof. In some examples, the method comprises applying the inner codec to encode the data as a plurality of polynucleotide sequences.

Methods provided herein may be carried out on a platform. In some instances, a platform comprise a hybrid organic-in silico platform. In some instances, the platform encodes data (e.g., binary data). In some instances, a platform comprises a computing system comprising at least one processor and instructions executable by the at least one processor to perform operations. In some instances, the operations comprise generating an inner codec comprising a codebook. In some instances, the codebook is generated with a base order. In some instances, the base order generates codewords with one or more base transitions. In some instances, the operations comprise applying the inner codec to encode the data as a plurality of polynucleotide sequences. In some instances, the platform comprises a synthesizer. In some instances, the platform comprises a synthesizer for generating a plurality of polynucleotides comprising the plurality of polynucleotide sequences. In some instances, the synthesizer generates a plurality of polynucleotide sequences by synthesis, ligation, assembly, or any combination thereof. In some instance a platform is integrated into one or more additional systems, such as traditional magnetic or tape storage devices.

### Nucleic Acid Based Information Storage

Provided herein are devices, compositions, systems and methods for nucleic acid-based information (data) storage. A biomolecule such as a DNA molecule provides a suitable host for information storage in-part due to its stability over time and capacity for enhanced information coding, as opposed to traditional binary information coding. In a first step, data comprising a first plurality of symbols, for example, a digital sequence encoding an item of information (i.e., digital information in a binary code for processing by a computer), is received. An encryption scheme is applied to convert the first plurality of symbols to a second plurality of symbols. The second plurality of symbols can comprise nucleic acid sequences. For example, an encryption scheme is applied to convert digital sequence from a binary code to a polynucleotide sequence. A surface material for nucleic acid extension, a design for loci for nucleic acid extension (aka, arrangement spots), and/or reagents for nucleic acid synthesis are selected. The surface of a structure is prepared for nucleic acid synthesis. De novo polynucleotide synthesis is then performed. The synthesized polynucleotides are stored and available for subsequent release, in whole or in part. Once released, the polynucleotides, in whole or in part, are sequenced, subject to decryption to convert nucleic sequence back to digital sequence. The digital sequence is then assembled to obtain an alignment encoding for the original item of information.

### Items of Information

Optionally, an early step of data storage process disclosed herein includes obtaining or receiving data comprising one or more items of information in the form of an initial code. Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, . PDF, .doc, .docx, .ppt, .pptx, .xls, .xlsx, .rtf, .jpg, .gif, .psd, .bmp, .tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes. In some instances, the digital information does not contain genomic data acquired from an organism. Items of information in some instances are encoded. Non-limiting encoding method examples include 1 bit/base, 2 bit/base, 4 bit/base or other encoding method.

### Methods and Systems for Information Storage

Provided herein are methods and systems for storing information (e.g., digital information). In some instances, provided herein are methods and systems for encoding. In some cases, the information comprises one or more objects. In some cases, the one or more objects comprises an item of information, such as, but not limited to, those described herein. In some cases, the one or more objects comprises a file or a metadata associated the file. In some cases, the methods and systems encode digital data, such as binary data. In some instances, the methods and systems comprise an inner codec, an outer codec, or a combination thereof. In some cases, the binary data comprises a byte stream or a byte array. In some cases, the data or the one or more objects is about 1 GB to about 1 TB. In some cases, the data is about 1 GB to about 1 TB. In some cases, the data or the one or more objects is about 1 GB to about 10 GB, about 1 GB to about 50 GB, about 1 GB to about 100 GB, about 1 GB to about 500 GB, about 1 GB to about 1 TB, about 10 GB to about 50 GB, about 10 GB to about 100 GB, about 10 GB to about 500 GB, about 10 GB to about 1 TB, about 50 GB to about 100 GB, about 50 GB to about 500 GB, about 50 GB to about 1 TB, about 100 GB to about 500 GB, about 100 GB to about 1 TB, or about 500 GB to about 1 TB. In some cases, the data is about 1 GB, about 10 GB, about 50 GB, about 100 GB, about 500 GB, or about 1 TB. In some cases, the data or the one or more objects is at least about 1 GB, about 10 GB, about 50 GB, about 100 GB, or about 500 GB. In some cases, the data or the one or more objects is at most about 10 GB, about 50 GB, about 100 GB, about 500 GB, or about 1 TB.

A system of storing digital information can comprise one or more processing units, a memory in communication with the one or more processing units, instructions stored in the memory and executed on the one or more processing units, or any combination thereof. In some cases, the one or more processing units and memory are distributed across one or more physical or logical locations. In some cases, the one or more processing units include any combination of central processing units (CPUs), graphical processing units (GPUs), single core processors, multi-core processors, processor clusters, application-specific integrated circuits (ASICs), programmable circuits such as Field Programmable Gate Arrays (FPGA), an AI-accelerator and variations thereof. In some cases, the one or more of the processing units comprise a Single Instruction Multiple Data (SIMD) or Single Program Multiple Data (SPMD) parallel architectures. As an example, the one or more processing units include one or more GPUs or CPUs that implement SIMD or SPMD. In some instances, an AI-accelerator comprise Google-TPU, Graphcore, Cerebras, SambaNova, or a combination thereof. In some embodiments, one or more of the processing units is implemented in software and/or firmware, in addition to hardware implementations. Software or firmware implementations of the processing units can include computer- or machine-executable instructions written in any suitable programming language to perform the various functions described herein. Software implementations of the one or more processing units can be stored in whole or part in the memory. Alternatively or additionally, the system can comprise one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. In some cases, the memory comprises removable storage, non-removable storage, local storage, and/or remote storage to provide storage of instructions, data structures, program modules (e.g., hashing module), and any other data described herein. In some instances, the memory is used to store information related to the algorithms described herein (e.g., software code, parameters, executable instructions, etc.).

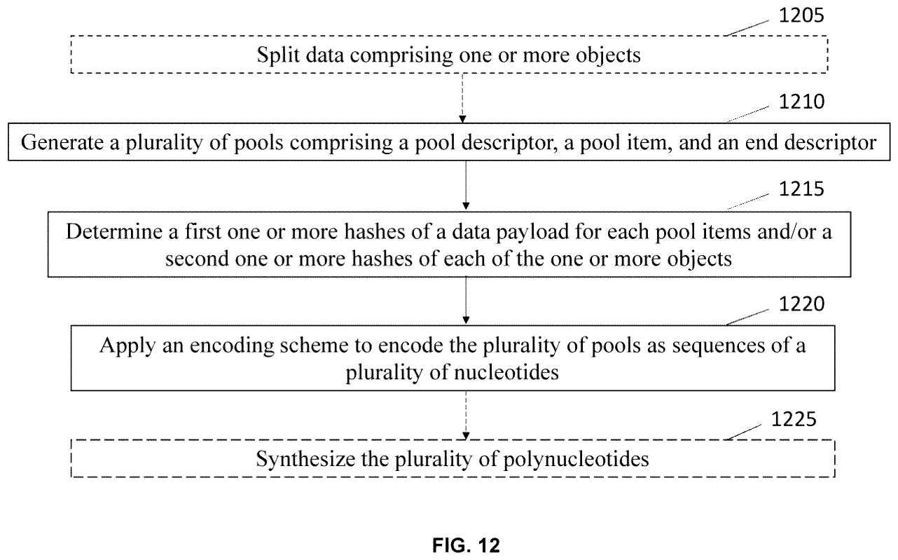

The instructions stored on the memory can comprise one or more steps for storing digital information. One or more operations for storing digital information is exemplary illustrated in FIG. 12. The dotted operations may be performed in some embodiments, but not in others. In some cases, the one or more steps comprises splitting digital information of one or more objects into a plurality of pools 1205. In some instances, an object of the one or more objects are split across more than one pool. In some cases, each of the plurality of pools is about 1 GB to about 1 TB. In some cases, each of the plurality of pools is about 1 GB to about 1 TB. In some cases, each of the plurality of pools is about 1 GB to about 10 GB, about 1 GB to about 50 GB, about 1 GB to about 100 GB, about 1 GB to about 500 GB, about 1 GB to about 1 TB, about 10 GB to about 50 GB, about 10 GB to about 100 GB, about 10 GB to about 500 GB, about 10 GB to about 1 TB, about 50 GB to about 100 GB, about 50 GB to about 500 GB, about 50 GB to about 1 TB, about 100 GB to about 500 GB, about 100 GB to about 1 TB, or about 500 GB to about 1 TB. In some cases, each of the plurality of pools is about 1 GB, about 10 GB, about 50 GB, about 100 GB, about 500 GB, or about 1 TB. In some cases, each of the plurality of pools is at least about 1 GB, about 10 GB, about 50 GB, about 100 GB, or about 500 GB. In some cases, each of the plurality of pools is at most about 10 GB, about 50 GB, about 100 GB, about 500 GB, or about 1 TB.

In some instances, the one or more objects comprises an item of information, such as a file, as previously described herein. In some instances, the one or more objects comprises a metadata associated with an item of information (e.g., metadata associated with a file). Non-limiting examples of metadata associated with an object include a list of keywords attached to an object, an object size, a thumbnail picture, a text summary, an ID range for a sorted key-value database, a timestamp, a version, or any other data providing information about one or more aspects of an object, or any combination thereof. In some examples, the metadata is customizable. In some examples, the metadata is used to search for an object in the plurality of pools.

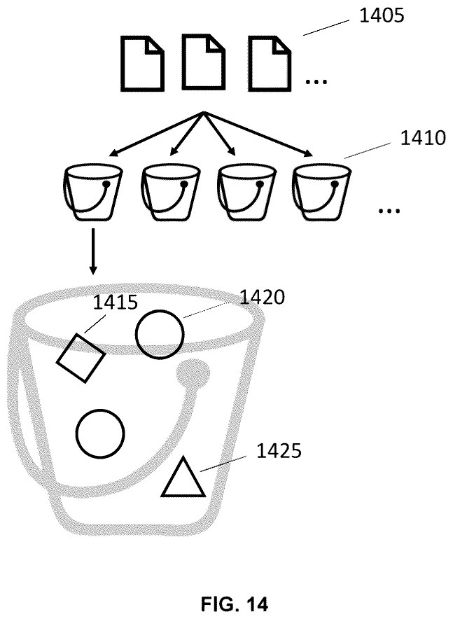

An exemplary diagram of digital information storage is illustrated in FIG. 14. As shown, one or more objects 1405 can be split into a plurality of pools 1410. In some cases, one object is split into a plurality of pools. In some cases, one object is split into a plurality of pools based in part on the size. In some cases, one object is split into two, three, four, five, six, seven, eight, nine, or ten pools. In some cases, more than one object is split into a plurality of pools. In some cases, one or more objects is in a pool. In some cases, one, two, three, four, five, six, seven, eight, nine, or ten objects are in a pool. In some cases, the plurality of pools are duplicated. In some cases, the plurality of pools comprise redundant pools, where two or more pools comprise the same one or more objects. In some cases, two, three, four, five, six, seven, eight, nine, or ten pools comprise the same one or more objects.

Each pool in the plurality of pools can comprise any one of or a combination of a pool descriptor, a pool item, or an end descriptor. In some cases, a pool comprises at least one pool item. In some cases, a pool comprises more than one pool item. In some cases, a pool comprises at least one pool descriptor. In some cases, a pool comprises more than one pool descriptor. In some cases, a pool comprises at least one end descriptor. In some cases, a pool comprises more than one end descriptor. As an example, each pool comprises a pool descriptor 1415, one or more pool items 1420, and an end descriptor 1425. In some cases, a pool comprises redundant pool items, pool descriptors, end pool descriptors, or a combination thereof. In such cases, two or more pool items, pool descriptors, end pool descriptors, or a combination thereof are identical. In some instances, two, three, four, five, six, seven, eight, nine, or ten, pool descriptors, end pool descriptors, or a combination thereof are identical.

Referring to FIG. 12, in some cases, the one or more operations in the instructions comprise generating a plurality of pools comprising a pool descriptor, a pool item, and an end descriptor 1210. In some instances, the data is divided into pools and the instructions comprise generating a pool descriptor, a pool item, an end descriptor, or any combination thereof in each pool of the plurality of pools. In such instances, the generated a pool descriptor, a pool item, an end descriptor are added to each of the pools. In some cases, the pool descriptor comprises a version, a pool ID, a list of pool item descriptors, or any combination thereof. In some instances, the version comprises the version of information (e.g., if information is updated). In some instances, the version is the version of the structure of the pool. In some instances, the version enables changing the overall pool structure for different file systems.

In some instances, the pool ID comprises a unique ID of the pool. In some examples, the unique ID comprises a universal unique identifier (UUID). In some examples, the unique ID comprises a content ID. In some examples, the content ID comprises a digital fingerprinting system, which can be used to identify and/or manage copyright or ownership of a content. In some instances, the list of pool item descriptors comprises a path of an object, a size of an object (e.g., a total size of an object), a range of the pool item within an object, offset of the pool item in a pool, or any combination thereof. In some examples, the range of the pool item within an object comprises one or more locations of a payload in the pool item within an object. In some examples, the one or more locations comprises a start and/or an end range of a payload in a pool item (e.g., line 1-6 in pool item 1, line 7-13 in pool item 2, . . , etc., in a pool). In some examples, the offset of the pool item comprises a payload location of the first byte of each of the one or more pool items in the payload of a pool. For example, the offset of the first pool item is 0 bytes. If the range of the first pool item is 1000-2000, then its size is 1000 bytes. In such an example, the offset of the next pool item will be 1000 bytes. In some cases, the pool item comprises a data payload and/or a hash of the pool item. In some instances, the data payload comprises the object or a portion of the object that is being stored. In some instances, the hash of the pool item comprises a hashed value of the object or a portion of the object that is being stored. In some cases, the end pool descriptor comprises a list of object descriptors. In some instances, the list of object descriptors comprises a path of the object and/or a hash of the object. In some examples, the path of the object comprises a unique path. In some examples, the path of the object comprises a hierarchy (e.g., directory hierarchy). In some examples, the path of the object does not comprise a hierarchy.

The systems and methods for storing digital information can comprise one or more hashes. In some cases, the one or more hashes are determined using a hashing module. In some cases, the hashing module is executed on the one or more processing units, such as those described herein. In some cases, the hashing module comprises instructions for determining the one or more hashes (e.g., a hash function). In some cases, the instructions (e.g., a hash function) are stored on a memory, such as those described herein. In some cases, information comprising an object, a part of an object, or a pool item is stored using a hash. In some cases, a first one or more hashes of data payloads of each of a one or more pool items is determined and/or a second one or more hashes of each of a one or more objects is determined 1215. In some instances, the data payload comprises an object or part of an object. In some instances, a hash of a pool item is appended to the data payload. In some instances, a hash of an object is appended to the end pool descriptor.

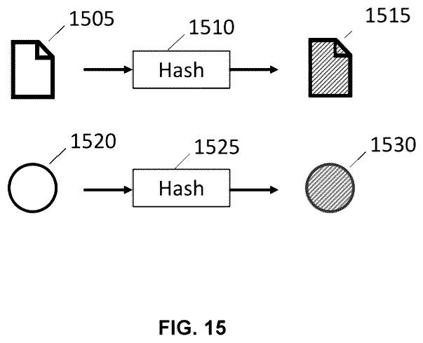

A hash may be determined a hash function (FIG. 15). A hash function generally comprises a function that turns an input of arbitrary length into an output with a fixed length (e.g., 224, 256, 384, 512 bits or characters). In some cases, the hash function comprises a cryptographic hash function. In some cases, the hash function comprises MD-5, SHA-1, SHA-2, SHA-3, RIPEMD-160, Whirlpool, BLAKE, BLAKE2, BLAKE3, or a variation thereof. In some instances, the hash function comprises SHA-2. In some examples, SHA-2 comprises SHA-224, SHA-256, SHA-384, SHA-512, SHA-512/224, or SHA-512/256. The output of a hash function can be deterministic and infeasible to reverse-engineer. Further, generating an output of fixed length can increase security, since any party involved in decrypting a hash would not be able to tell the length of the input. In some examples, a hash is generated upon inputting an identification code, encryption key, password, or any variation thereof. In some examples, the hash allows verification of the content (e.g., item of information or digital information stored in a pool) during decoding.

In some cases, the input 1505 comprises an object. In some examples, a hash function 1510 is used to determine a hashed output (or hash) 1515. In some cases, the input 1520 comprises an object. In some examples, a hash function 1525 is used to determine a hashed output (or hash) 1530. In some examples, the hash function 1510 and hash function 1525 are the same hash function. In some examples, the hash function 1510 and hash function 1525 are both SHA-256. In some examples, the hash function 1510 and hash function 1525 are different hash functions. In some examples, the output 1515 and the output 1530 are the same length. In some examples, the output 1515 and the output 1530 are both 256 bits. In some examples, the output 1515 and the output 1530 are different lengths.

A hash function can comprise one or more operations to generate a hash. In some cases, the one or more steps in a hash function comprises padding bits. In some instances, extra bits are added to the digital information (or the message) being hashed. In some examples, extra bits are added to the message such that the length of the digital message is a modulus value less than a total number of bits. In some examples, the modulus value is 64 bits. In some examples, the number of bits is 512 bits and the length of the digital information is 448 bits (e.g., for SHA-256). In some examples, the first extra bit comprises a binary digit of 1. In some examples, the subsequently added extra bits comprise a binary digit of 0s.

In some cases, the one or more steps in a hash function comprises padding a length. In some instances, padding the length comprises adding a modulus value to the digital information (e.g., also referred to as a bi-endian (BE) integer). The modulus value or the BE integer generally represents the length of the original input comprising the original digital information in binary. In some examples, the modulus value is 64 bits. In some examples, 64 bits are added to the digital message of 448 bits, and the total number of bits is 512 bits (e.g., for SHA-256). In some instances, the modulus value is calculated by applying a modulus to the original digital information. As an example, if the original digital information is “hello world” in binary, the length of the original input is 88 bits, which is “1011000” in binary. As such, 0s followed by “1011000” are added to the end of the 448 bits of digital information such that the total number of bits is 512.

In some cases, the one or more steps in the hash function comprises initializing one or more hash values or buffers. In some instances, 8 hash values or buffers are initialized. In some instances, the initialized hash values are hard-coded (e.g., constants). In some instances, the initialized hash values represent a first 32 bits of fractional part of the square roots of the first 8 primes (e.g, 2, 3, 5, 7, 11, 13, 17, 19). In some cases, the one or more steps in the hash function further comprises initializing round constants (or keys). In some instances, 64 round constants are initialized. In some examples, each of the 64 round constants represent the first 32 bits of the fractional parts of the cube roots of the first 64 primes (e.g., 2-311). In some instances, the 64 different round constants are stored in an array.

In some cases, the one or more steps in the hash function comprises compression. In some instances, each block of information (e.g., every 512 bits) undergoes compression. During compression, each block of information undergoes a fixed number of rounds. In some instances, the number of rounds in 64. In some instances, compression is performed by a one-way compression function. In some instances, the one-way compression function is single block-length compression function. In some examples the compression function is a Davies-Meyer, Matyas-Meyer-Oseas, or Miyaguchi-Preneel compression function. In some instances, the one-way compression function is double block-length compression function. In some examples the compression function is a MDC-2/Meyer-Schilling, MDC-4, or Hirose compression function. In some instances, the output from the compression function is less than the block of information. In some examples, the output has a length of 256 bits.

In some cases, one or more of the hashes (e.g., hashes of pool item(s), hashes of object(s)) are calculated during storage of information. In some cases, all of the hashes (e.g., hashes of pool item(s), hashes of object(s)) are calculated during storage of information. In some examples, this allows stable low memory usage regardless of the size of the objects. In some cases, the first one or more hashes of data payloads of each pool item requires less memory than the one or more objects. In some cases, the second one or more hashes of each of the one or more objects require less memory than one or more pool items. In some cases, the source data (e.g., item of information) is read only once. In some cases, each of the pools are written once without seeks. In some examples, this minimizes data transfers and latency.

In some cases, the hashes described herein can serve one or more purposes. The one or more purposes can comprise, by way of non-limiting example, one or more of: verifying the integrity of one or more items of information (e.g., an object), signature generation and verification (e.g., for digital signatures), password verification, proof-of-work, or identifier for item of information.

In some cases, an encryption and/or compression can further be added. In some examples, the encryption and/or compression is implemented with streaming application programmable interface (API). In some examples, this avoids the need to store intermediate results. In some cases, the digital information to be stored is already compressed, for example, to reduce data transfer costs. In some cases, the digital information to be stored is already encrypted, for example, for security reasons.

The one or more operations in the instructions stored on the memory can further comprise creating a plurality of index pools. In some cases, the plurality of index pools contain only indices. In some cases, the index pools are used when retrieving the objects stored in the plurality of pools encoded in a plurality of polynucleotides. In some instances, index pools are sequenced and temporarily stored in digital storage systems (e.g. flash drives) to search for objects. In some examples, once a pool is identified, the plurality of polynucleotides encoding the pool is sequenced.

In some cases, the one or more index pools comprise an index pool descriptor and/or a list of object indexing. In some instances, the index pool descriptor comprises a version, a pool ID, a size of a pool, a timestamp, or a combination thereof. In some examples, the pool ID comprises a unique ID of the pool. In some examples, the unique ID comprises a universal unique identifier (UUID). In some examples, the unique ID comprises a content ID. In some examples, the content ID comprises a digital fingerprinting system, which can be used to identify and/or manage copyright or ownership of a content. In some examples, the size of each of the plurality of index pools is about 1 GB to about 1 TB. In some instances, the list of an object indexing comprises a path of an object, a hash of an object, a list of object fragments, a list of object metadata, or any combination thereof. In some examples, the path of the object comprises a unique path. In some examples, the path of the object comprises a hierarchy (e.g., directory hierarchy). In some examples, the path of the object does not comprise a hierarchy. In some examples, the hash of the object is a hash as previously described herein (e.g., SHA-256). In some examples, the list of object fragments comprises a pool ID of a pool containing a fragment, a range of a fragment, or a combination thereof. In some examples, the list of object metadata comprises the type of metadata, the metadata payload, or a combination thereof. In some examples, the type of metadata comprises, a list of keywords attached to an object, a thumbnail picture, a text summary, an ID range for a sorted key-value database, a timestamp, a version, or any combination thereof. In some examples, the metadata is customizable. In some examples, the metadata is used to search for an object in the plurality of pools.

In some cases, an index pool can store information of about 1 to about 1 million pools. In some cases, an index pool can store information of about 1 pool to about 10 pools, about 1 pool to about 100 pools, about 1 pool to about 1,000 pools, about 1 pool to about 5,000 pools, about 1 pool to about 10,000 pools, about 1 pool to about 50,000 pools, about 1 pool to about 100,000 pools, about 1 pool to about 500,000 pools, about 1 pool to about 1 million pools, about 10 pools to about 100 pools, about 10 pools to about 1,000 pools, about 10 pools to about 5,000 pools, about 10 pools to about 10,000 pools, about 10 pools to about 50,000 pools, about 10 pools to about 100,000 pools, about 10 pools to about 500,000 pools, about 10 pools to about 1 million pools, about 100 pools to about 1,000 pools, about 100 pools to about 5,000 pools, about 100 pools to about 10,000 pools, about 100 pools to about 50,000 pools, about 100 pools to about 100,000 pools, about 100 pools to about 500,000 pools, about 100 pools to about 1 million pools, about 1,000 pools to about 5,000 pools, about 1,000 pools to about 10,000 pools, about 1,000 pools to about 50,000 pools, about 1,000 pools to about 100,000 pools, about 1,000 pools to about 500,000 pools, about 1,000 pools to about 1 million pools, about 5,000 pools to about 10,000 pools, about 5,000 pools to about 50,000 pools, about 5,000 pools to about 100,000 pools, about 5,000 pools to about 500,000 pools, about 5,000 pools to about 1 million pools, about 10,000 pools to about 50,000 pools, about 10,000 pools to about 100,000 pools, about 10,000 pools to about 500,000 pools, about 10,000 pools to about 1 million pools, about 50,000 pools to about 100,000 pools, about 50,000 pools to about 500,000 pools, about 50,000 pools to about 1 million pools, about 100,000 pools to about 500,000 pools, about 100,000 pools to about 1 million pools, or about 500,000 pools to about 1 million pools. In some cases, an index pool can store information of about 1 pool, about 10 pools, about 100 pools, about 1,000 pools, about 5,000 pools, about 10,000 pools, about 50,000 pools, about 100,000 pools, about 500,000 pools, or about 1 million pools. In some cases, an index pool can store information of at least about 1 pool, about 10 pools, about 100 pools, about 1,000 pools, about 5,000 pools, about 10,000 pools, about 50,000 pools, about 100,000 pools, or about 500,000 pools. In some cases, an index pool can store information of at most about 10 pools, about 100 pools, about 1,000 pools, about 5,000 pools, about 10,000 pools, about 50,000 pools, about 100,000 pools, about 500,000 pools, or about 1 million pools.

In some cases, each of the one or more index pools is about 1 GB to about 1 TB. In some cases, each of the plurality of pools is about 1 GB to about 1 TB. In some cases, each of the one or more index pools is about 1 GB to about 10 GB, about 1 GB to about 50 GB, about 1 GB to about 100 GB, about 1 GB to about 500 GB, about 1 GB to about 1 TB, about 10 GB to about 50 GB, about 10 GB to about 100 GB, about 10 GB to about 500 GB, about 10 GB to about 1 TB, about 50 GB to about 100 GB, about 50 GB to about 500 GB, about 50 GB to about 1 TB, about 100 GB to about 500 GB, about 100 GB to about 1 TB, or about 500 GB to about 1 TB. In some cases, each of the one or more index pools is about 1 GB, about 10 GB, about 50 GB, about 100 GB, about 500 GB, or about 1 TB. In some cases, each of the one or more index pools is at least about 1 GB, about 10 GB, about 50 GB, about 100 GB, or about 500 GB. In some cases, each of the one or more index pools is at most about 10 GB, about 50 GB, about 100 GB, about 500 GB, or about 1 TB.

An encoding scheme can be applied to each of the plurality of pools and/or index pools. In some cases, the encoding scheme encodes the digital information in the plurality of pools as a plurality of polynucleotides 1220. In some cases, the encoding scheme encodes the digital information in the index pools as a plurality of polynucleotides. In some instances, the encoding scheme comprises codecs for encoding binary data as polynucleotide sequences (e.g., inner codec). In some instances, the encoding scheme comprises an error correction code (ECC). In some cases, the encoding scheme (e.g., inner codec or low-level codec) is also designed and implemented to allow streaming read and write API access. In some cases, the encoding scheme (e.g., inner codec or low-level codec) is also designed and implemented to match the streaming of the systems and methods for digital storage (e.g., high-level codec) described herein.

The encoding scheme can generally comprise one or more operations. The one or more operations can comprise one or more operation to manipulate or transform data (e.g., digital information). The one or more operations can comprise by way of non-limiting example, splitting, shuffling, concatenating, transposing, translating, duplicating, labeling (e.g., using an index) data or a part of the data, or any combination thereof.

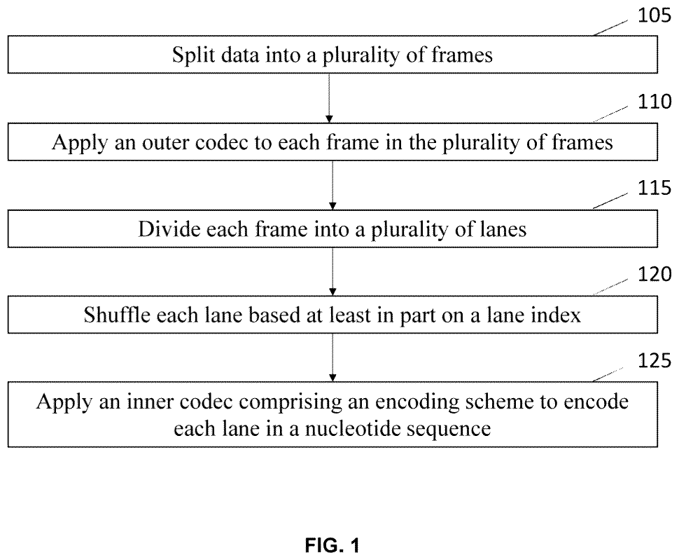

A method of encoding digital information (e.g., binary data) in a plurality of polynucleotide sequences is schematically illustrated in FIG. 1. In some instances, methods for encoding digital or data in a plurality of polynucleotide sequences comprises splitting the data. In some instances, the data is split into a plurality of frames 105. In some instances, the plurality of frames comprise about 100 to about 10,000 frames. In some instances, the plurality of frames comprise about 100 frames to about 250 frames, about 100 frames to about 500 frames, about 100 frames to about 750 frames, about 100 frames to about 1,000 frames, about 100 frames to about 2,500 frames, about 100 frames to about 5,000 frames, about 100 frames to about 7,500 frames, about 100 frames to about 10,000 frames, about 250 frames to about 500 frames, about 250 frames to about 750 frames, about 250 frames to about 1,000 frames, about 250 frames to about 2,500 frames, about 250 frames to about 5,000 frames, about 250 frames to about 7,500 frames, about 250 frames to about 10,000 frames, about 500 frames to about 750 frames, about 500 frames to about 1,000 frames, about 500 frames to about 2,500 frames, about 500 frames to about 5,000 frames, about 500 frames to about 7,500 frames, about 500 frames to about 10,000 frames, about 750 frames to about 1,000 frames, about 750 frames to about 2,500 frames, about 750 frames to about 5,000 frames, about 750 frames to about 7,500 frames, about 750 frames to about 10,000 frames, about 1,000 frames to about 2,500 frames, about 1,000 frames to about 5,000 frames, about 1,000 frames to about 7,500 frames, about 1,000 frames to about 10,000 frames, about 2,500 frames to about 5,000 frames, about 2,500 frames to about 7,500 frames, about 2,500 frames to about 10,000 frames, about 5,000 frames to about 7,500 frames, about 5,000 frames to about 10,000 frames, or about 7,500 frames to about 10,000 frames. In some instances, the plurality of frames comprise about 100 frames, about 250 frames, about 500 frames, about 750 frames, about 1,000 frames, about 2,500 frames, about 5,000 frames, about 7,500 frames, or about 10,000 frames. In some instances, the plurality of frames comprise at least about 100 frames, about 250 frames, about 500 frames, about 750 frames, about 1,000 frames, about 2,500 frames, about 5,000 frames, or about 7,500 frames. In some instances, the plurality of frames comprise at most about 250 frames, about 500 frames, about 750 frames, about 1,000 frames, about 2,500 frames, about 5,000 frames, about 7,500 frames, or about 10,000 frames. In some cases, the frames each comprise the same amount of data. In alternative cases, the frames may each comprise a different amount of data. In some instances, each frame is assigned a frame index. In some examples, the frame index increases for each frame index (e.g., 0, 1, 2, 3, 4, 5, . . . , etc.). In some examples, the frame index monotonically increases for each frame index.

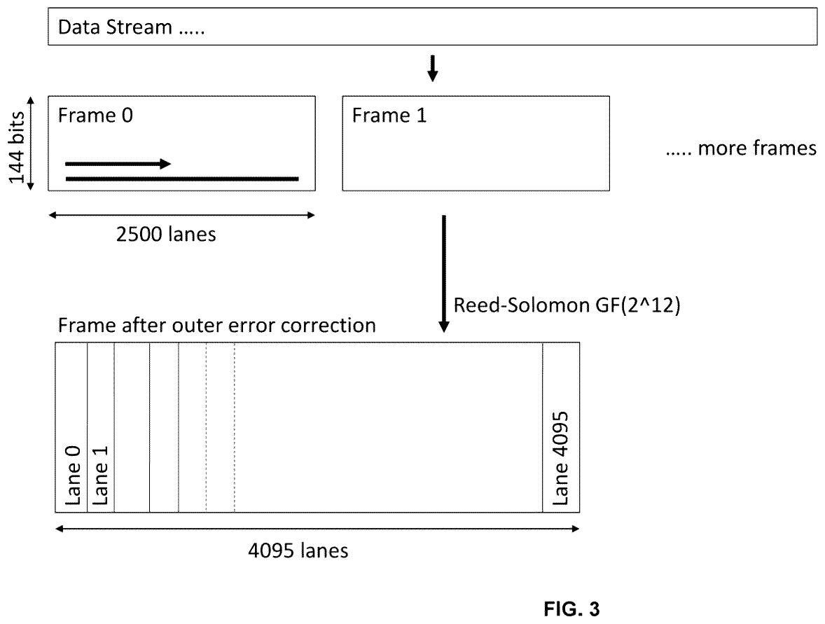

Methods for encoding digital or binary data comprise an outer codec. In some instances, methods for encoding digital or binary data in a plurality of polynucleotide sequences comprise an outer codec. In some instances, an outer codec is applied to the data (e.g., binary data). In some instances, an outer codec is applied to the data once the data is split into a plurality of frames 110. In such instances, outer codec is applied to each of the plurality of frames. An exemplary diagram of splitting a data stream into frames and applying an outer codec is exemplary illustrated in FIG. 3.

In some instances, the outer codec comprises an error correction scheme or an error correction code (ECC), such as a Reed-Solomon (RS) code. This outer codec is used for spreading the digital or binary data to be stored over many oligonucleotides. In some instances, spreading the data builds redundancy, which can be used to correct for erasures (e.g., lost oligos). In some further instances, spreading the data also builds redundancy to correct errors from an inner codec.

In some instances, the error correction scheme comprises Reed-Solomon (RS) code. In such instances, a RS encoder is used to encode the binary data or plurality of frames comprising binary data. Generally the RS codes operates on a block of data treated as a set of finite-field elements. In some instances, the RS code comprises mapping data, e.g., x=(x₁, . . . , xₖ) ∈ F^{k}, to a polynomial pₓ, where

$${p}_{x} ( a ) = {\sum }_{i = 1}^{k} {x}_{i} {a}^{i - 1} .$$

The encoded data C(x) is obtained by evaluating pₓ at various different n points a₁, . . . , aₙ in the field F (e.g., C(x)=pₓ(a₁), . . . , pₓ (aₙ)).

In some further embodiments, the RS code comprises an encoding scheme in which each codeword contains the message as a prefix, and error correcting symbols are appended as a suffix. In some instances, the RS code is specified as RS (n, k) with m-bit symbols. In such instances, the encoder takes k data symbols of m-bits each, and adds parity symbols (error correcting symbols or check symbols) to make an n symbol codeword. Here, there is n-k parity symbols (or check symbols, t) of m bits each. In some cases, the RS decoder corrects up to t symbols that contain errors in a codeword, where 2t=n−k. The codeword C(x) comprises the parity check information CK(x) which is systematically appended to the message information M(x). The codeword C(x) can be calculated as: C (x)=x^{n-k}M(x)+CK(x)=x^{n-k}M(x)+x^{n-k}M(x) mod g(x). Here, k refers to the message length (e.g., symbols), t refers to the number of errors to be corrected, n refers to the block length (e.g., message length n plus the correction length t), and m refers to the symbol width, where given the symbol size, m, the maximum codeword length n for RS code is n=2^{m}−1. Further, x^{n-k} refers to the displacement shift in the message, and g(X) refers to the generator polynomial, which is defined as the polynomial whose roots are sequential powers of the Galois field (GF) primitive a (e.g., g(x)=(x−aⁱ)(x−^{a+1}) . . . (x−a^{i+n-k-1})=g₀+g₁x+ . . . +gₙ₋ₖ₋₁xⁿ⁻¹⁻¹+x^{n-k}).

For example, in RS (255, 223) with 8-bit symbols, the block length n is 255 codeword bytes, the message length k is 223 bytes, and the parity 2t is 32 bytes. In such an example, the RS decoder corrects up to 16 symbol errors in the codeword, meaning errors up to 16 bytes can be corrected by the decoder. The RS code can also be denoted based on the Galois Field as GF (2^{m}). For example, in an RS GF (2¹²) encoding scheme, as shown in FIG. 3, n is 4095 (e.g., n=2¹²−1=4096−1=4095). If k is, for example 2499, then 2t=4095-2499=1596 and t is thus 798.

In some instances, the error correction scheme comprises a linear error correction code (or linear block code), such as a low-density parity-check (LDPC) code. In some cases, the error correction scheme comprises a linear block error-correcting code, such as polar code. In some further embodiments, the error correction scheme comprises a high-performance forward error correction (FEC), such as a Turbo-code. In some instances, the error correction scheme comprises an RS code, an LDPC code, a Turbo-code, a polar code, or any combination thereof (e.g., RS-based LDPC codes).

In some instances, the error correction scheme comprises low density parity check (LDPC) code. In such instances, the LDPC code is used to encode the binary data or plurality of frames comprising binary data. Generally the structure of a LDPC code is defined by a parity check matrix containing 0s at most entries and 1s elsewhere. For instance, an (N, K) LDPC code for K information bits is a linear block code with a block size of N, defined by a sparse (N−K)×N parity check matrix in which all elements other than 1 s are 0s. The number of 1s in a row or a column is referred to as the degree of the row or the column. In some instances, a codeword of length Nis represented as a vector C and for information bits of length K, an (N, K) code with 2K codewords is used. In some instances, the (N, K) LDPC code is defined by an (N−K)×N parity check matrix H, satisfying the condition: HC^{T}=0.

In some instances, the LDPC code is regular when each row and each column of the parity check matrix has a constant degree and irregular otherwise. In some instances, an irregular LDPC code outperforms a regular LDPC code. In some instances, due to different degrees among rows and among columns, the irregular LDPC code promises improved performance only if the row degrees and the column degrees are appropriately adjusted.

In some instances, the error correction scheme comprises a polar code. In some instances, a polar code can achieve Shannon capacity by theoretical proof. In some instances, a polar code comprises low encoding and decoding complexity. A polar code generally comprises a generator matrix G_{N}, and information can be encoded according to xI^{N}=ul^{N}G^{N}, where xₗ^{N} is an output bit after encoding, uₗ^{N} is an input bit before encoding, and the generator matrix is defined as G^{N}=B_{N}F^{⊗n}. The code length N is defined as N=2n, where n≥0. B_{N} comprises a transposed matrix such as, for example, a bit reversal matrix. F^{⊗n} comprises a Kronecker power of F, which is defined as F^{⊗n}=F^{⊗}F^{⊗n-1}, where

$$F = [ \begin{matrix}1 & 0 \\ 1 & 1\end{matrix} ] .$$

In some instances, the polar code is represented as (N, K, A, U_{A}^{c}) with a cosec code, and the encoding process is defined as xI^{N}=U_{A}G_{N}(A)⊗u_{A}^{c}G_{N}(A^{c}), where A is an information bit index set, G_{N} (A) is a submatrix obtained from a row, which corresponds to the index in the set A, in G_{N}. Further, G_{N} (A^{c}) is a submatrix obtained from a row, which corresponds to the index in the set A^{c}, in G_{N}, and u_{A}^{c} is frozen bits the number of which is (N−K), with N being the code length and K being the length of information bits. In some instances, the frozen bit is set to 0, and the above encoding process is described as xₗ^{N}=u_{A}G_{N}(A).

In some instances, the error correction scheme comprises a turbo code. A turbo code generally comprises the parallel concatenation of two or more component codes applied to different interleaved versions of the same information sequence. Generally, recursive systematic convolutional (RSC) codes are used as the component codes. The structure of a turbo code, for example, comprises two RSC encoders (e.g., M=2), concatenated in parallel, and the code rate R is R=⅓, since R=1/(M+1) (approximately). The input to the first RSC encoder is the original information sequence. The original information sequence d is also applied to an interleaver to produce an interleaved version d′. The interleaved version d′ of the information sequence is the input to the second RSC encoder. The outputs from the turbo encoder comprise systematic sequences of u and redundant parts x₍₁₎ (output from the first RSC encoder) and x₍₂₎ (output from the second encoder). Therefore, the output of the encoder comprises u₁, x₁₍₁₎, x₁₍₂₎, u₂, x₂₍₁₎, x₂₍₂₎, where uₖ is the k^{th} systematic bit (i.e., data bit), xₖ₍₁₎ is the parity output from the first RSC encoder associated with the k^{th} systematic bit uₖ; and xₖ₍₂₎ is the parity output from the second RSC encoder associated with the k^{th} systematic bit uₖ. The decoding procedure for the turbo codes generally comprises iterative decoding. The turbo code decoding procedure can comprise two component decoders (corresponding to two RSC encoders), an interleaver; and a de-interleaver. In some instances, the two component decoders are soft-input and soft-output (SISO) decoders. In some instances, outputs of the two component decoders comprise likelihood information concerning the coded data sequence.

In some instances, the size of the data is increased once an outer codec is applied. In some instances, the frame sizes are increased once an outer codec is applied to each of the frame comprising data. In some instances, the frames are divided into a plurality of lanes 115. In some instances, each lane comprises a lane index. In some cases, each frame comprises about 1000 to about 10,000 lanes. In some cases, each frame comprises about 5000 lanes. In some cases, each frame comprises about 1,000 lanes to about 2,500 lanes, about 1,000 lanes to about 5,000 lanes, about 1,000 lanes to about 7,500 lanes, about 1,000 lanes to about 10,000 lanes, about 2,500 lanes to about 5,000 lanes, about 2,500 lanes to about 7,500 lanes, about 2,500 lanes to about 10,000 lanes, about 5,000 lanes to about 7,500 lanes, about 5,000 lanes to about 10,000 lanes, or about 7,500 lanes to about 10,000 lanes. In some cases, each frame comprises about 1,000 lanes, about 2,500 lanes, about 5,000 lanes, about 7,500 lanes, or about 10,000 lanes. In some cases, each frame comprises at least about 1,000 lanes, about 2,500 lanes, about 5,000 lanes, or about 7,500 lanes. In some cases, each frame comprises at most about 2,500 lanes, about 5,000 lanes, about 7,500 lanes, or about 10,000 lanes. Each lane can further comprise about 100 to about 300 bits. In some cases, each lane comprises about 100 bits to about 150 bits, about 100 bits to about 200 bits, about 100 bits to about 250 bits, about 100 bits to about 300 bits, about 150 bits to about 200 bits, about 150 bits to about 250 bits, about 150 bits to about 300 bits, about 200 bits to about 250 bits, about 200 bits to about 300 bits, or about 250 bits to about 300 bits. In some cases, each lane comprises about 100, 110, 120, 130, 140, 150, 160, 170, 180, 190, 200, 210, 220, 230, 240, 250, 260, 270, 280, 290, 300 bits. In some cases, each lane comprises at least about 100, 110, 120, 130, 140, 150, 160, 170, 180, 190, 200, 210, 220, 230, 240, 250, 260, 270, 280, 290, 300 bits. In some cases, each lane comprises at most about 100, 110, 120, 130, 140, 150, 160, 170, 180, 190, 200, 210, 220, 230, 240, 250, 260, 270, 280, 290, 300 bits. While the methods for encoding provided herein are illustrated for example using binary data, in some instances, the methods may be generally applied to data comprising a plurality of symbols.

In some instances, the methods for encoding data in a plurality of polynucleotide sequences comprise shuffling the data. In some instances, each lane is shuffled base at least in part on the lane indices 120. In some instances, each lane is shuffled after applying an outer codec to the binary data. In some cases, shuffling each lane allows resistance against errors that can occur during synthesis or sequencing, such as those affecting a whole oligonucleotide pool. The errors can comprise an insertion, a deletion, a substitution, or a combination thereof. In some instances, the shuffling comprises a rotation scheme within each lane based partly on each lane index. For example, each bit in a lane may be shifted by each lane index (e.g., no shuffling in lane 0, 1 bit shift in lane 1, 2 bit shift in lane 2, etc.).

In some further instances, the shuffling comprises a pseudorandom process within each lane. In this pseudorandom shuffling process, a random seed are used to initialize a pseudorandom number generator. In some instances, a number generated by the pseudorandom number generator is determined by the random seed. Therefore, the same sequence of numbers are generated by the pseudorandom number generator using the same seed. As an example, using shuffling comprises a pseudorandom process, each bit in a lane is be shifted according to the numbers generated by the pseudorandom number generator.

In some further instances, the lane index is used as a seed to create a permutation of some or all the bits for that lane. In some instances, the permutation of the some or all the bits is created by sampling from a random number generator. In some instances, the permutation is stored in a pre-compiled form. In some instances, the use of a pseudo random generator allows for a smaller implementation source code.

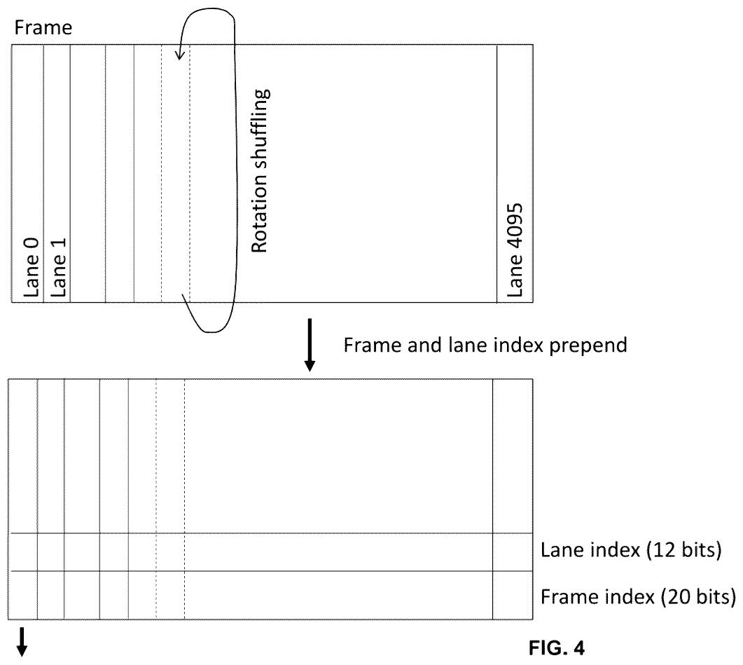

In some instances, the frame index and the lane index are prepended. In some instances, the frame index and the lane index are prepended to each lane once each lane is shuffled. An exemplary diagram of shuffling the lanes and prepending the frame index and the lane index is shown in FIG. 4. In some cases, the frame index comprises about 12 bits to about 20 bits. In some cases, the frame index comprises about 12 bits to about 14 bits, about 12 bits to about 16 bits, about 12 bits to about 18 bits, about 12 bits to about 20 bits, about 14 bits to about 16 bits, about 14 bits to about 18 bits, about 14 bits to about 20 bits, about 16 bits to about 18 bits, about 16 bits to about 20 bits, or about 18 bits to about 20 bits. In some cases, the frame index comprises about 12 bits, about 14 bits, about 16 bits, about 18 bits, or about 20 bits. In some cases, the frame index comprises at least about 12 bits, about 14 bits, about 16 bits, or about 18 bits. In some cases, the frame index comprises at most about 14 bits, about 16 bits, about 18 bits, or about 20 bits. In some cases, the lane index comprises about 12 bits to about 16 bits. In some cases, the lane index comprises about 12 bits to about 14 bits, about 12 bits to about 16 bits, or about 14 bits to about 16 bits. In some cases, the lane index comprises about 12 bits, about 14 bits, or about 16 bits. In some cases, the lane index comprises at least about 12 bits, or about 14 bits. In some cases, the lane index comprises at most about 14 bits, or about 16 bits. As shown in FIG. 4, in some instances, the lane index is 12 bits and the frame index is 20 bits. In some cases, the lane index is the symbol width m from the RS code.

In some instances, the methods for encoding data in a plurality of polynucleotide sequences comprise an inner codec. In some instances, the inner codec is applied to the data (e.g., binary data). In some instances, the inner codec is applied to the data from the outer codec. In some instances, the inner codec is applied to the lanes of the data. In some instances, the inner codec is applied to the lanes of the data once the lanes have been shuffled.

In some instances, the inner codec comprises an encoding scheme. In some instances, an inner codec comprising an encoding scheme is applied to each lane to encode the data as a polynucleotide sequence 125. The inner codec is used to transform data (e.g., digital or binary data) into nucleotide bases. In some instances, the inner codec is capable of correcting errors such as deletion, substitution, or insertion errors, or any combination thereof. In some further embodiments, the inner codec is used to validate oligos and discard any suspicious oligos to avoid contaminating the outer decoding. The inner codec further encodes the indices (frame index and lane index), which can allow for efficient clustering during decoding.

In some instances, the encoding scheme adds redundancy across the plurality of polynucleotide sequences. In some instances, the redundancy is about 5% to about 10%. In some instances, the redundancy is about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some instances, the redundancy is about 5%, about 6%, about 7%, about 8%, about 9%, or about 10%. In some instances, the redundancy is at least about 5%, about 6%, about 7%, about 8%, or about 9%. In some instances, the redundancy is at most about 6%, about 7%, about 8%, about 9%, or about 10%. In some cases, this redundancy allows a pool of oligos to be decoded in the presence of errors in the individual oligos, such as insertions, deletions, substitutions, or any combination thereof.

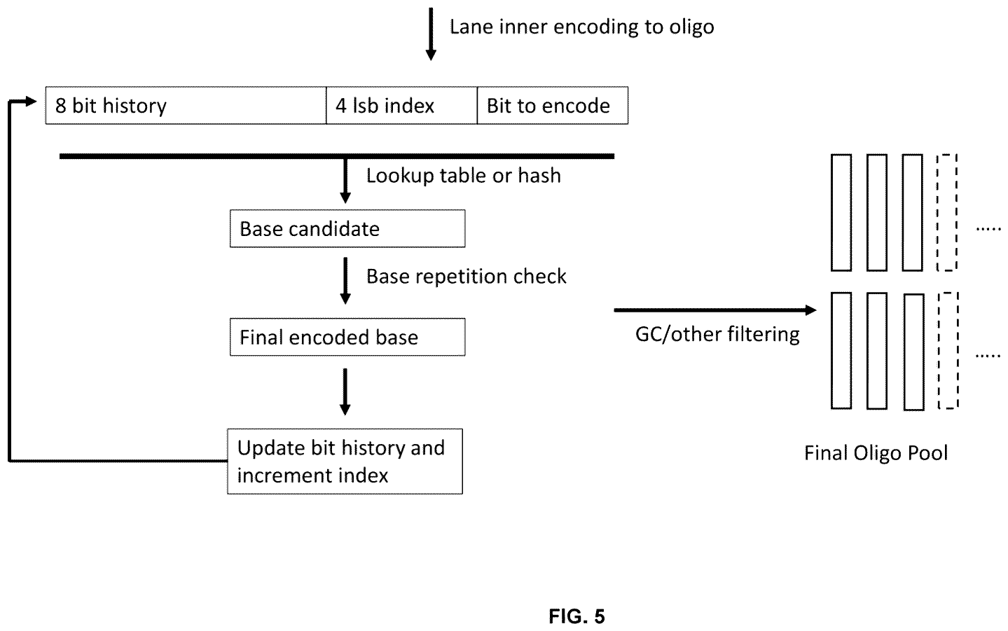

An exemplary diagram of an encoding scheme is shown in FIG. 5. In this exemplary diagram, the encoding scheme in the inner codec combines two or more of: bits from each lane, a bit history, and a bit position. In some instances, a model (e.g., adaptive model) is used to partition known bits into a context, and each context is mapped to a bit history. In some cases, the bit history is represented by an 8-bit state. In some instances, the bit history is updated each time a context is encountered, for example, through the use of a lookup table. A bit position comprises a fixed number of least significant bits (LSBs). In some instances, the LSBs comprise a bit index of the bits to encode. For example, if 100 bits encode a 100-mer oligonucleotide, a “bit index” refers to an index from 0 to 99 in the bits to encode. For example, if 100 bits encode a 100-mer oligonucleotide, a “bit index” refers to an index from 0 to 99 in the bits to encode. The LSB comprises the bit position in a binary integer representing the binary 1s place of the integer. In some instances, the LSB index is any length. In some instances, the LSB index is represented by a 2-bit state, a 3-bit state, or a 4-bit state. As an example, an index 0, 1, 2, 3, 4, 5, 6, 7, . . . can be represented as a 2-bit state 00, 01, 10, 11, 00, 01, 10, 11, . . . , respectively. While the encoding scheme illustrated in FIG. 5 comprises binary data, in some instances, the encoding scheme may be generally applied to data comprising a plurality of symbols.

In some instances, the inner codec comprises generating base candidates for bits of the binary data. Base candidates are generated for the binary data using a lookup table, a hash, or a combination thereof. In some instances, the hash is determined using methods previously described herein. In some instances, the binary data comprises two or more of: bits from each lane, bit history, and a bit position. In some instances, the bit rate for encoding is about 1 bit per base to about 2 bits per base. In some instances, the bit rate for encoding is about 1 bit per base to about 1.1 bits per base, about 1 bit per base to about 1.2 bits per base, about 1 bit per base to about 1.3 bits per base, about 1 bit per base to about 1.4 bits per base, about 1 bit per base to about 1.5 bits per base, about 1 bit per base to about 1.6 bits per base, about 1 bit per base to about 1.7 bits per base, about 1 bit per base to about 1.8 bits per base, about 1 bit per base to about 1.9 bits per base, about 1 bit per base to about 2 bits per base, about 1.1 bits per base to about 1.2 bits per base, about 1.1 bits per base to about 1.3 bits per base, about 1.1 bits per base to about 1.4 bits per base, about 1.1 bits per base to about 1.5 bits per base, about 1.1 bits per base to about 1.6 bits per base, about 1.1 bits per base to about 1.7 bits per base, about 1.1 bits per base to about 1.8 bits per base, about 1.1 bits per base to about 1.9 bits per base, about 1.1 bits per base to about 2 bits per base, about 1.2 bits per base to about 1.3 bits per base, about 1.2 bits per base to about 1.4 bits per base, about 1.2 bits per base to about 1.5 bits per base, about 1.2 bits per base to about 1.6 bits per base, about 1.2 bits per base to about 1.7 bits per base, about 1.2 bits per base to about 1.8 bits per base, about 1.2 bits per base to about 1.9 bits per base, about 1.2 bits per base to about 2 bits per base, about 1.3 bits per base to about 1.4 bits per base, about 1.3 bits per base to about 1.5 bits per base, about 1.3 bits per base to about 1.6 bits per base, about 1.3 bits per base to about 1.7 bits per base, about 1.3 bits per base to about 1.8 bits per base, about 1.3 bits per base to about 1.9 bits per base, about 1.3 bits per base to about 2 bits per base, about 1.4 bits per base to about 1.5 bits per base, about 1.4 bits per base to about 1.6 bits per base, about 1.4 bits per base to about 1.7 bits per base, about 1.4 bits per base to about 1.8 bits per base, about 1.4 bits per base to about 1.9 bits per base, about 1.4 bits per base to about 2 bits per base, about 1.5 bits per base to about 1.6 bits per base, about 1.5 bits per base to about 1.7 bits per base, about 1.5 bits per base to about 1.8 bits per base, about 1.5 bits per base to about 1.9 bits per base, about 1.5 bits per base to about 2 bits per base, about 1.6 bits per base to about 1.7 bits per base, about 1.6 bits per base to about 1.8 bits per base, about 1.6 bits per base to about 1.9 bits per base, about 1.6 bits per base to about 2 bits per base, about 1.7 bits per base to about 1.8 bits per base, about 1.7 bits per base to about 1.9 bits per base, about 1.7 bits per base to about 2 bits per base, about 1.8 bits per base to about 1.9 bits per base, about 1.8 bits per base to about 2 bits per base, or about 1.9 bits per base to about 2 bits per base. In some instances, the bit rate for encoding is about 1 bit per base, about 1.1 bits per base, about 1.2 bits per base, about 1.3 bits per base, about 1.4 bits per base, about 1.5 bits per base, about 1.6 bits per base, about 1.7 bits per base, about 1.8 bits per base, about 1.9 bits per base, or about 2 bits per base. In some instances, the bit rate for encoding is at least about 1 bit per base, about 1.1 bits per base, about 1.2 bits per base, about 1.3 bits per base, about 1.4 bits per base, about 1.5 bits per base, about 1.6 bits per base, about 1.7 bits per base, about 1.8 bits per base, or about 1.9 bits per base. In some instances, the bit rate for encoding is at most about 1.1 bits per base, about 1.2 bits per base, about 1.3 bits per base, about 1.4 bits per base, about 1.5 bits per base, about 1.6 bits per base, about 1.7 bits per base, about 1.8 bits per base, about 1.9 bits per base, or about 2 bits per base. In some instances, the lookup table is used to map bits to nucleotides (e.g., A=00, T=10, C=01, G=11). In some instances, a hash comprises a function that can be used to map data of an arbitrary size (e.g., arbitrary number bits) to a fixed size value (e.g., a nucleotide or hashed value). In some examples, the hashed value is mapped to polynucleotide sequences.

In some instances, the inner codec comprises a base repetition check. In some instances, the base repetition check is performed once the base candidates are selected. In some instances, the base repetition check checks for repetitions in two or more sequential bases. In some instances, the base repetition check substitutes one base for another if there are repetition in two or more sequential bases. In some instances, the lookup table or the hash is updated based on bases that were updated during the base repetition check. Further, after the base repetition check, the bit history is updated. In some instances, the frame index and/or lane index are incremented. In some instances, this process is repeated until sequences of all of the plurality of polynucleotide sequences are determined.

In some instances, the inner codec further comprises performing GC filtering prior to synthesizing the plurality of the polynucleotide sequences. In some cases, the GC filtering removes about 1% to about 10% of lanes in the plurality of lanes. In some cases, the GC filtering removes about 5% to about 10% of lanes in the plurality of lanes. In some cases, the GC filtering removes no lanes in the plurality of lanes. In some cases, the GC filtering removes about 1%, about 2%, about 3%, about 4%, about 5%, about 6%, about 7%, about 8%, about 9%, or about 10%. In some cases, the GC filtering removes at least about 1%, about 2%, about 3%, about 4%, about 5%, about 6%, about 7%, about 8%, or about 9%. In some cases, the GC filtering removes at most about 2%, about 3%, about 4%, about 5%, about 6%, about 7%, about 8%, about 9%, or about 10%. In some cases, the plurality of polynucleotide sequences comprises about 40% to about 60% GC content. In some cases, the plurality of polynucleotide sequences comprises about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60% GC content. In some cases, the plurality of polynucleotide sequences comprises about 40%, about 45%, about 50%, about 55%, or about 60% GC content. In some cases, the plurality of polynucleotide sequences comprises at least about 40%, about 45%, about 50%, or about 55% GC content. In some cases, the plurality of polynucleotide sequences comprises at most about 45%, about 50%, about 55%, or about 60% GC content. In some cases, at least 90% of the plurality of polynucleotide sequences comprises about 40% to about 60% GC content. In some cases, at least 90% of the plurality of polynucleotide sequences comprises about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60% GC content. In some cases, at least 90% of the plurality of polynucleotide sequences comprises about 40%, about 45%, about 50%, about 55%, or about 60% GC content. In some cases, at least 90% of the plurality of polynucleotide sequences comprises at least about 40%, about 45%, about 50%, or about 55% GC content. In some cases, at least 90% of the plurality of polynucleotide sequences comprises at most about 45%, about 50%, about 55%, or about 60% GC content. In some cases, the output from the inner codec comprises a final oligonucleotide pool.

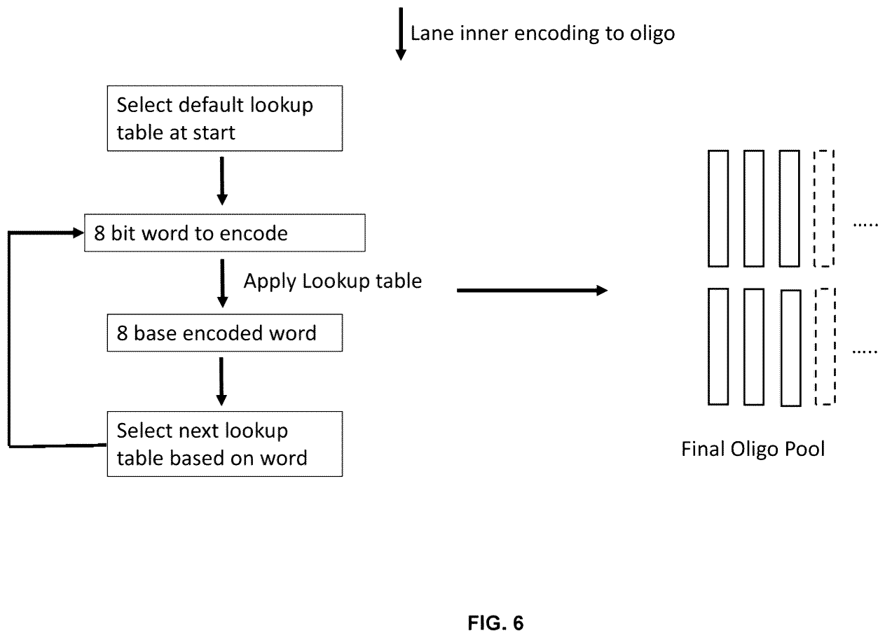

An exemplary diagram of an alternative encoding scheme is shown in FIG. 6. In some instances, the encoding scheme in the inner codec comprises starting with a default lookup table. The default lookup table is used to select a word to encode within each lane. In some instances, the word comprises a plurality of symbols. In some examples, the word is an 8 bit word or a byte. The lookup table is applied to generate base candidates for each word or byte) within each lane. A next lookup table is selected based on the previously encoded word or byte. In some instances, the encoding scheme further comprises performing a base repetition check, GC filtering, or a combination thereof, as previously described herein. In some instances, this process is repeated until sequences of all of the plurality of polynucleotide sequences may be determined. In some cases, the output from the inner codec comprises a final oligonucleotide pool or a final oligonucleotide library.

In some cases, the length of each of the oligonucleotides (or polynucleotides) in a library is about 20 to about 500 bases. In some cases, the length of each of the oligonucleotides (or polynucleotides) in a library is about 20 bases to about 50 bases, about 20 bases to about 100 bases, about 20 bases to about 200 bases, about 20 bases to about 300 bases, about 20 bases to about 400 bases, about 20 bases to about 500 bases, about 50 bases to about 100 bases, about 50 bases to about 200 bases, about 50 bases to about 300 bases, about 50 bases to about 400 bases, about 50 bases to about 500 bases, about 100 bases to about 200 bases, about 100 bases to about 300 bases, about 100 bases to about 400 bases, about 100 bases to about 500 bases, about 200 bases to about 300 bases, about 200 bases to about 400 bases, about 200 bases to about 500 bases, about 300 bases to about 400 bases, about 300 bases to about 500 bases, or about 400 bases to about 500 bases. In some cases, the length of each of the oligonucleotides (or polynucleotides) in a library is about 20 bases, about 50 bases, about 100 bases, about 200 bases, about 300 bases, about 400 bases, or about 500 bases. In some cases, the length of each of the oligonucleotides (or polynucleotides) in a library is at least about 20 bases, about 50 bases, about 100 bases, about 200 bases, about 300 bases, or about 400 bases. In some cases, the length of each of the oligonucleotides (or polynucleotides) in a library is at most about 50 bases, about 100 bases, about 200 bases, about 300 bases, about 400 bases, or about 500 bases.

In some instances, the methods to encode data in a plurality polynucleotide sequences, as described herein, are performed on a system. In some instances, such a system comprise an apparatus comprising a memory, a processing device operatively coupled to the memory, or a combination thereof. In some instances, the memory is used to store information of the binary data, the polynucleotide sequences, or the combination thereof. In some instances, the information of the data (e.g., binary data), the polynucleotide sequences, or the combination thereof is from one or more step in the encoding methods described herein. In some instances, the memory is used to store information related to the algorithms described herein (e.g., software code, parameters, executable instructions, etc.). In some examples, the memory can comprise any suitable memory described herein. In some examples, the memory can be configured according to embodiments described herein.

In some instances, the processing device is configured to perform one or more encoding steps. In some instances, the processing device is configured to perform one or more operations comprising: split the data into a plurality of frames; apply an outer codec to each frame in the plurality of frames; divide each frame into a plurality of lanes; shuffling each lane based at least in part on the lane index; and apply an inner codec comprising an encoding scheme to encode each lane in a polynucleotide sequence. In some instances, each frame in the plurality of frames comprises a frame index. In some instances, each lane in the plurality of lanes comprises a lane index. In some instances, the outer codec comprising an error correction scheme. In some instances, the encoding scheme adds redundancy so that the binary data can be decoded in the presence of an error in the polynucleotide sequence.

Methods, systems, and platforms for encoding data can comprise an inner codec optimized for one or more constraints. The one or more constraints can be related, by way of non-limiting example, nucleic acid synthesis, post-processing, storage, or sequencing. In some instances, nucleic acid synthesis comprises electrochemical synthesis, enzymatic synthesis, phosphoramidite synthesis, inkjet printing, or any combination thereof. In some instances, the one or more constraints related to nucleic acid synthesis comprises a synthesis error, such as an insertion, deletion, or mutation. In some instances, post-processing comprises one or more of ligation, cleavage, hybridization, denaturation, fixation to a solid support, extension, error correction, enrichment, isolation, purification, and amplification. In some instances, storage comprises cold data storage. Cold data storage may generally refer to storage of data, for example, in nucleic acids, that is rarely accessed. Cold data storage may be the opposite of “hot storage” referring to data that is frequently accessed. In some examples, storage comprises hot storage, in which data stored in nucleic acids are frequently accessed. In some instances, storage comprises nucleic acid storage in a liquid phase or solid phase. In some examples, one or more constraints related to storage comprises temperature (e.g., room temperature), humidity, pressure, salinity, pH, concentration, time, light, UV, O₂, or any combination thereof. In some instances, sequencing comprises next generation sequencing, parallel sequencing, single-molecule real-time sequencing, nanopore sequencing, sequencing by synthesis, Sanger sequencing, or any combination thereof.

Methods, systems, and platforms for encoding data can comprise an inner codec optimized for generation of polynucleotides. In some instances, generation of polynucleotides comprises assembly of polynucleotides. In some instances, generation of polynucleotides comprises synthesis of polynucleotides. Synthesis may comprise methods and system described herein, or any suitable methods and systems known in the art. In some instances, the data comprises one or more symbols. In some instances, the data comprises a string of symbols or a sequence of symbols. In some instances, the one or more symbols comprise binary data. In some instances, an inner codec is applied to the data. In some instances, an inner codec is applied to data from an outer codec (e.g., error correction scheme), such as those provided herein. In some instances, the inner codec is applied to unencrypted data. In some instances, the inner codec is applied to encrypted data. An inner codec may be optimized to generate polynucleotides following a specific order of bases. In some instances, this allows for more efficient synthesis of polynucleotides as the total number of synthesis cycles is reduced compared to the number of synthesis cycles needed to synthesize polynucleotides whose sequences are not encoded using an inner codec provided herein (e.g., unoptimized synthesis approach). In some instances, this allows for lower error rates as the number of oxidation step and deprotection steps during synthesis is reduced.

Provided herein is a method for encoding data. In some instances, the method comprises generating an inner codec comprising a codebook. The codebook may be optimized based on an application, manipulation, operation, or usage, of nucleic acids encoding data. The codebook may be optimized based on one of more constraints (e.g., related to nucleic acid synthesis, post-processing, storage, sequencing, etc.), as described herein. The codebook may be generated with a base order. In some instances, the codebook comprises codewords that are generated based in-part on the base order. In some instances, the base order comprises predetermined base transitions. In some instances, the codebook generates a polynucleotide sequence by mapping data represented by one or more symbols (e.g., binary “0”s and “1” s) to another one or more symbols, such as nucleic acids (e.g., A, T, C, G), using the codewords. In some instances, specific or predetermined base transitions allow for synthesis according to a base order. In some examples, pattern repeats are reduced by varying the synthesis order at each layer. Non-limiting examples of a synthesis order at a given layer can comprise [A, G, C, T], [C, A, T, G], [T, G, A, C], or any other combination of bases, A, T, G, C. In such examples, the codebook is varied for each layer. In some examples, two consecutive layers do not have the same codebook. In some examples, each layer comprises a unique codebook. In some examples, two or more layers comprise the same codebook.

In some examples, pattern repeats are reduced by only allowing for specific base transitions at each base. For example, after adenine (A), only guanine (G), cytosine (C), or thymine (T) can be selected as the next base in a sequence. Alternatively, no base is selected after A. In some examples, if G is selected, only C or T can be selected, or alternatively no base is selected. In some examples, if C is selected, only T can be selected, or alternatively no base is selected.

In some instances, the codebook comprises one, two, three, four, or five nucleotides. In some instances, the codebook comprises at least one, two, three, or four nucleotides. In some instances, the codebook comprises at most two, three, four, or five nucleotides. In some instances, the codebook comprises four nucleotides (e.g., adenine (A), thymine (T), cytosine (C), guanine (G)). For example, the specific base transitions for one or more layers comprise any one of: (a) [A, T, C, G], (b) [A, T, G, C], (c) [A, G, T, C], (d) [A, G, C, T], (e) [A, C, G, T], (f) [A, C, T, G], (g) [T, C, G, A], (h) [T, C, A, G], (i) [T, G, A, C], (j) [T, G, C, A], (k) [T, A, G, C], (1) [T, A, C, G], (m) [C, G, A, T], (n) [C, G, T, A], (o) [C, A, G, T], (p) [C, A, T, G], (q) [C, T, A, G], (r) [C, T, G, A], (s) [G, A, T, C], (t) [G, A, C, T], (u) [G, T, A, C], (v) [G, T, C, A], (w) [G, C, T, A], (x) [G, C, A, T], or (y) any combination thereof. In some instances, the specific base transitions for one or more layers comprise natural or canonical bases. In some instances, the specific base transitions for one or more layers comprise nucleotides with natural or canonical bases and one or more nucleotides with unnatural or non-canonical bases. As an example, a codebook can comprise a synthesis order according to repeats of [A, G, C, T] (e.g., A, G, C, T, A, G, C, T, . . . ). In such an example, the codebook can comprise the following codewords: A, G, C, T, AG, AC, AT, GC, GT, AGC, ACT, and AGCT. In some instances, the codewords in the codebook can be synthesized with a number of cycles equivalent to the number of nucleotides in the codebook. In some instances, the codewords in the codebook can be synthesize with 1, 2, 3, 4, or 5 cycles of synthesis. In some instances, the codewords in the codebook can be synthesize with at least 1, 2, 3, 4, or 5 cycles of synthesis. In some instances, the codewords in the codebook can be synthesize with at most 1, 2, 3, 4, or 5 cycles of synthesis. In some instances, transitions associated with a codebook are non-random or pseudo non-random. In some instances, transitions associated with a codebook are defined by a pre-defined mathematical algorithm or statistical algorithm.

In some instances, a synthesis order can be varied for one or more layers. A layer can generally comprise a flow of each base in a specific or predetermined order. For example, if the base transition is [A, T, C, G], a layer comprises a flow of A, followed by a flow of T, C, and then G during synthesis. In some instances, the one or more layers can comprise any one of: (a) [A, T, C, G], (b) [A, T, G, C], (c) [A, G, T, C], (d) [A, G, C, T], (e) [A, C, G, T], (f) [A, C, T, G], (g) [T, C, G, A], (h) [T, C, A, G], (i) [T, G, A, C], (j) [T, G, C, A], (k) [T, A, G, C], (1) [T, A, C, G], (m) [C, G, A, T], (n) [C, G, T, A], (o) [C, A, G, T], (p) [C, A, T, G], (q) [C, T, A, G], (r) [C, T, G, A], (s) [G, A, T, C], (t) [G, A, C, T], (u) [G, T, A, C], (v) [G, T, C, A], (w) [G, C, T, A], (x) [G, C, A, T], or (y) any combination thereof. In some instances, one or more of the specific base transitions of a layer can be repeated more than once. As an example, the synthesis order can comprise [A, G, C, T], [C, A, T, G], [T, G, A, C] . . . and the sequence can comprise AGCTAGCTCATGTGAC . . . , where the first layer is repeated twice. In some instances, varying the one or more layers reduces pattern repeats in the sequence (e.g., repetitive bases, high GC/AT, or secondary structures).

In some instances, the inner codec comprises one or more codebooks. In some instances, the inner codec comprises one, two, three, four, five, six, seven, eight, nine, or ten codebooks. In some instances, the inner codec comprises at least one, two, three, four, five, six, seven, eight, nine, or ten codebooks. In some instances, the inner codec comprises at most one, two, three, four, five, six, seven, eight, nine, or ten codebooks. In some instances, each codebooks encodes a layer during synthesis of the polynucleotides. In some instances, each codebook is generated with a unique base order. In some instances, each codebook is optimized for one or more base transitions. In some instances, a unique base order generates one or more unique base transitions. In some instances, each codebook is optimized for specific base transitions at a given layer, cycle index, history, or any combination thereof. In some examples, the history comprises one or more of the previous layers, the one or more codebooks encoding the previous one or more layers, the cycle indices of the one or more previous layers, or any combination thereof. In some instances, each codebook is generated by a pre-defined mathematical algorithm or statistical algorithm.

In some instances, the codebook comprises one or more nucleotide analogs or unnatural/non-canonical nucleotides. A nucleotide analog, or unnatural nucleotide, comprises a nucleotide which contains some type of modification. A nucleotide analog, or unnatural nucleotide, comprises a nucleotide which contains some type of modification to either the base, sugar, or phosphate moieties. A modification can comprise a chemical modification. Modifications may be, for example, of 3′OH or 5′OH group, of the backbone, of the sugar component, or of the nucleotide base. Modifications may include addition of non-naturally occurring linker molecules and/or of interstrand or intrastrand cross links. In one aspect, the modified nucleic acid comprises modification of one or more of 3′H or 5′OH group, the backbone, the sugar component, or the nucleotide base, and/or addition of non-naturally occurring linker molecules. In one aspect a modified backbone comprises a backbone other than a phosphodiester backbone. In one aspect a modified sugar comprises a sugar other than deoxyribose (in modified DNA) or other than ribose (modified RNA). In one aspect a modified base comprises a base other than adenine, guanine, cytosine or thymine (in modified DNA) or a base other than adenine, guanine, cytosine or uracil (in modified RNA).

The nucleic acid may comprise at least one modified base. Modifications to the base moiety include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA). Further examples of modified bases may be found for example in WO2019/014267 and US2022/0243244, which are incorporated herein by reference in its entirety.

In some embodiments, the codebook comprises one or more canonical nucleotides and one or more non-canonical nucleotides. In some instances, the canonical nucleotides comprise one or more of A, T, C, G, or U. In some instances, the non-canonical nucleotides comprise one or more nucleotide analogs or unnatural nucleotides provided herein. In some instances, the non-canonical nucleotides comprise one or more canonical nucleotides with a modification. In some instances, the codebook comprises about one, two, three, four, or five canonical nucleotides. In some instances, the codebook comprises about one, two, three, four, or five non-canonical nucleotides. In some instances, the codebook comprises about at least one, two, three, four, or five canonical nucleotides. In some instances, the codebook comprises about at least about one, two, three, four, or five non-canonical nucleotides. In some instances, the codebook comprises at most about one, two, three, four, or five canonical nucleotides. In some instances, the codebook comprises about at most about one, two, three, four, or five non-canonical nucleotides. In some instances, the codebook comprises any combination of canonical and non-canonical nucleotides, such as those provided herein.

In some instances, a codebook comprises about 1 to about 30 codewords. In some instances, the codebook comprises about 1 to about 5, about 1 to about 10, about 1 to about 12, about 1 to about 15, about 1 to about 18, about 1 to about 20, about 1 to about 22, about 1 to about 25, about 1 to about 28, about 1 to about 30, about 5 to about 10, about 5 to about 12, about 5 to about 15, about 5 to about 18, about 5 to about 20, about 5 to about 22, about 5 to about 25, about 5 to about 28, about 5 to about 30, about 10 to about 12, about 10 to about 15, about 10 to about 18, about 10 to about 20, about 10 to about 22, about 10 to about 25, about 10 to about 28, about 10 to about 30, about 12 to about 15, about 12 to about 18, about 12 to about 20, about 12 to about 22, about 12 to about 25, about 12 to about 28, about 12 to about 30, about 15 to about 18, about 15 to about 20, about 15 to about 22, about 15 to about 25, about 15 to about 28, about 15 to about 30, about 18 to about 20, about 18 to about 22, about 18 to about 25, about 18 to about 28, about 18 to about 30, about 20 to about 22, about 20 to about 25, about 20 to about 28, about 20 to about 30, about 22 to about 25, about 22 to about 28, about 22 to about 30, about 25 to about 28, about 25 to about 30, or about 28 to about 30 codewords. In some instances, the codebook comprises about 1, about 5, about 10, about 12, about 15, about 18, about 20, about 22, about 25, about 28, or about 30 codewords. In some instances, the codebook comprises at least about 1, about 5, about 10, about 12, about 15, about 18, about 20, about 22, about 25, or about 28 codewords. In some instances, the codebook comprises at most about 5, about 10, about 12, about 15, about 18, about 20, about 22, about 25, about 28, or about 30 codewords.

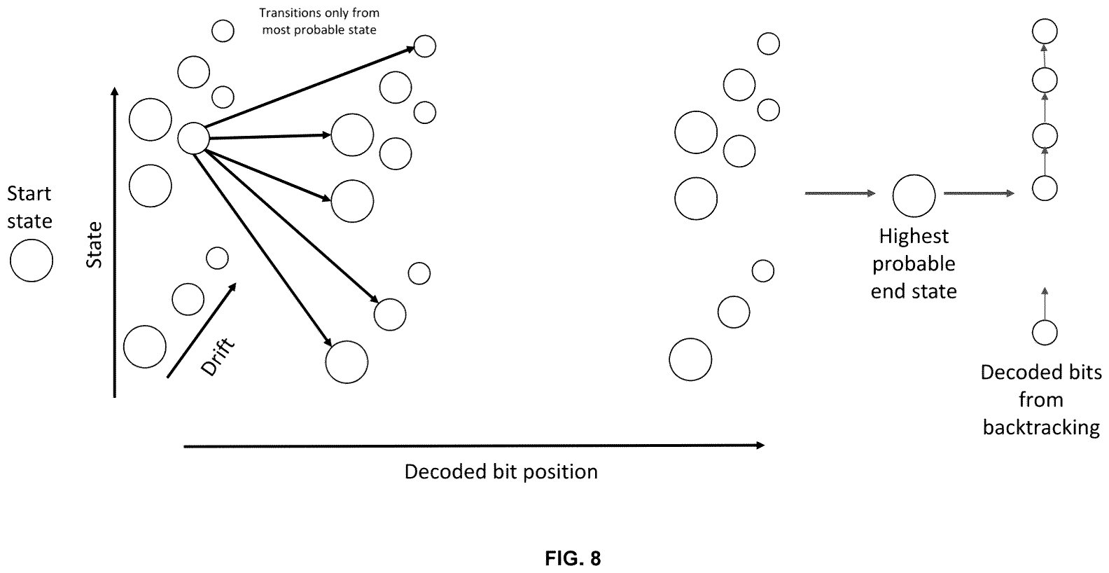

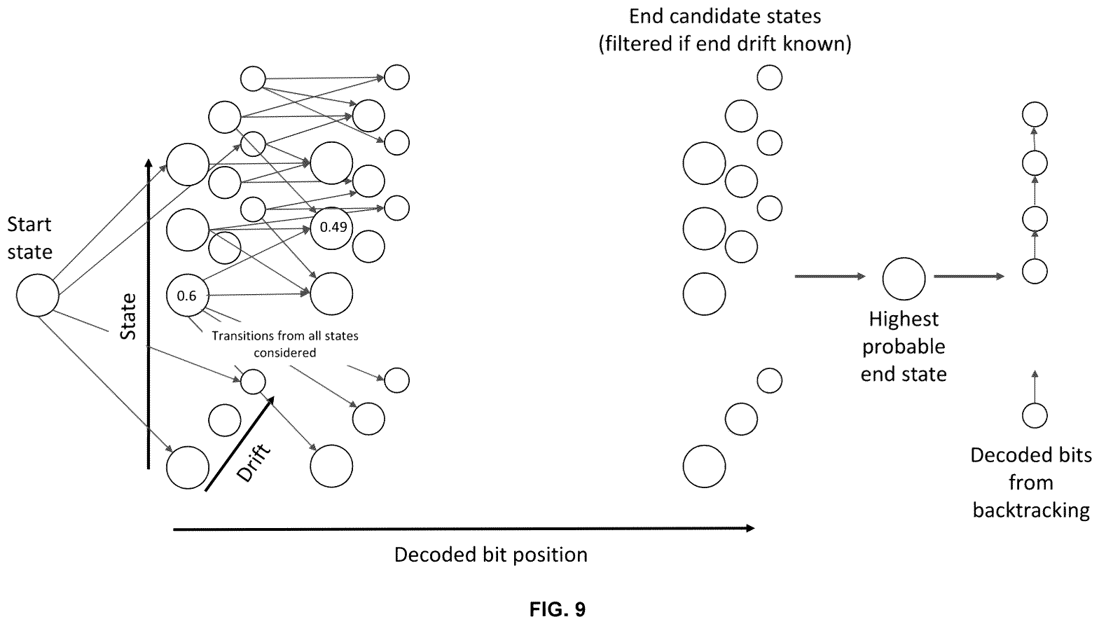

The inner codec comprising the codebook can be applied to encode the data as a plurality of polynucleotide sequences. In some instances, the data comprises digital data. In some instances, the data comprises one or more symbols. In some instances, the one or more symbols are mapped to a plurality of polynucleotide sequences based on the codebook. For example, a numerical value, such as binary digits (e.g., sequence(s) of 0 or 1), can be mapped to a codeword in the codebook. In some instances, the inner codec is further optimized against one or more constraints. The one or more constraints can comprise a constraint related to the plurality of polynucleotide sequences. In some examples, the one or more constraints comprise a length of the plurality of polynucleotide sequences. In some examples, the one or more constraints comprise GC content of the plurality of polynucleotide sequences. In some examples, the one or more constraints comprise base repeats of the plurality of polynucleotide sequences. In some examples, the one or more constraints comprise one or more errors, such as an insertion, mutation, or deletion. In some instances, mapping binary data to codewords creates a graph of transitions. The transitions can comprise one or more transitions between codewords and codebooks based on the values of binary data and the location (e.g., index). In some instances, one or more probabilities are calculated based on estimated deletion, insertion, and/or mutation rates during decoding. In some instances, a decoding algorithm finds one or more solutions to maximize the transition probability, as provided herein (e.g., FIG. 8 and FIG. 9).

In some instances, a portion of the plurality of polynucleotide sequences encode for redundancy. In some instances, the portion of the plurality of polynucleotide sequences that encode for redundancy is about 20% to about 80%. In some instances, the portion of the plurality of polynucleotide sequences that encode for redundancy is about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80%. In some instances, the portion of the plurality of polynucleotide sequences that encode for redundancy is about 20%, about 30%, about 40%, about 50%, about 60%, about 70%, or about 80%. In some instances, the portion of the plurality of polynucleotide sequences that encode for redundancy is at least about 20%, about 30%, about 40%, about 50%, about 60%, or about 70%. In some instances, the portion of the plurality of polynucleotide sequences that encode for redundancy is at most about 30%, about 40%, about 50%, about 60%, about 70%, or about 80%.

In some instances, the plurality of polynucleotide sequences are the same length. In some-instances, about 70% to about 100% of the plurality of polynucleotide sequences have a same length. In some instances, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100% of the plurality of polynucleotide sequences have a same length. In some instances, about 70%, about 75%, about 80%, about 85%, about 90%, about 95%, or about 100% of the plurality of polynucleotide sequences have a same length. In some instances, at least about 70%, about 75%, about 80%, about 85%, about 90%, or about 95% of the plurality of polynucleotide sequences have a same length. In some instances, at most about 75%, about 80%, about 85%, about 90%, about 95%, or about 100% of the plurality of polynucleotide sequences have a same length. In some instances, the plurality of polynucleotide sequences are different lengths. In some instances, the plurality of polynucleotide sequences differ by 1% to about 30%. In some instances, the plurality of polynucleotide sequences differ by about 1% to about 5%, about 1% to about 10%, about 1% to about 15%, about 1% to about 20%, about 1% to about 25%, about 1% to about 30%, about 5% to about 10%, about 5% to about 15%, about 5% to about 20% about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%. In some instances, the plurality of polynucleotide sequences differ by about 1%, about 5%, about 10%, about 15%, about 20%, about 25%, or about 30%. In some instances, the plurality of polynucleotide sequences differ by at least about 1%, about 5%, about 10%, about 15%, about 20%, or about 25%. In some instances, the plurality of polynucleotide sequences differ by at most about 5%, about 10%, about 15%, about 20%, about 25%, or about 30%.

A plurality of polynucleotides comprising the plurality of polynucleotide sequences can be generated. In some instances, the plurality of polynucleotides are synthesized. In some instances, synthesis comprises base-by-base synthesis. In some instances, synthesis comprises a synthesis cycle. A synthesis cycle generally refers to one or more steps performed to achieve a nucleotide coupling. A synthesis cycle can comprise one or more of: deblocking (or deprotecting), coupling, oxidation, and capping. In some instances, the synthesis comprises a number of synthesis cycles. The inner codec may allow for more efficient synthesis by reducing the number of synthesis cycles required. In some instances, the number of synthesis cycles required to synthesize a plurality of polynucleotides comprising a plurality of polynucleotide sequence encoded by the inner codec is reduced compared to the number of synthesis cycles required to synthesize a plurality of polynucleotides with sequences not encoded by the inner codec. In some instances, the number of synthesis cycles is reduced by about 5 to about 80%. In some instances, the number of synthesis cycles is reduced by about 5% to about 10%, about 5% to about 20%, about 5% to about 30%, about 5% to about 40%, about 5% to about 50%, about 5% to about 60%, about 5% to about 70%, about 5% to about 80%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80%. In some instances, the number of synthesis cycles is reduced by about 5%, about 10%, about 20%, about 30%, about 40%, about 50%, about 60%, about 70%, or about 80%. In some instances, the number of synthesis cycles is reduced by at least about 5%, about 10%, about 20%, about 30%, about 40%, about 50%, about 60%, or about 70%. In some instances, the number of synthesis cycles is reduced by at most about 10%, about 20%, about 30%, about 40%, about 50%, about 60%, about 70%, or about 80%. As an example, an inner codec with 12 codewords using 4 nucleotides encodes a plurality of polynucleotide sequences with about 50% redundancy. Therefore, in this example, 6 values of the binary data are mapped to the 12 codewords, which are equivalent to log 2 (12)=3.58 bits of information. However, taking into account redundancy, for example, 2× redundancy, the correspond to 3.58/2=1.79 bits of information per codeword. If the payload in each of the plurality of polynucleotide sequences is about 100 bits, this requires about 100 bits/1.79 bits per codeword=55.8 codewords. With the optimized inner codec and cycle ordering, as described herein, a codeword requires 4 cycles, resulting in about 55.8 codewords×4 cycles per codeword=about 224 cycles of synthesis. However, without the inner codec, the synthesis would require 400 cycles (e.g., 4×100). As a further example, if the payload in each of the plurality of polynucleotide sequences is about 300 bits, this requires about 447 cycles of synthesis (e.g., 200/1.79×4). However, without the inner codec, the synthesis would require 800 cycles (e.g., 4×200). As an additional example, if the payload in each of the plurality of polynucleotide sequences is about 300 bits, this requires about 670 cycles of synthesis (e.g., 300/1.79×4). However, without the inner codec, the synthesis would require 1200 cycles (e.g., 4×300).

In some instances, the plurality of polynucleotides are the same length. In some instances, about 70% to about 100% of the plurality of polynucleotides have a same length. In some instances, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100% of the plurality of polynucleotides have a same length. In some instances, about 70%, about 75%, about 80%, about 85%, about 90%, about 95%, or about 100% of the plurality of polynucleotides have a same length. In some instances, at least about 70%, about 75%, about 80%, about 85%, about 90%, or about 95% of the plurality of polynucleotides have a same length. In some instances, at most about 75%, about 80%, about 85%, about 90%, about 95%, or about 100% of the plurality of polynucleotides have a same length. In some instances, the plurality of polynucleotides are different lengths. In some instances, the plurality of polynucleotides differ by 1% to about 30%. In some instances, the plurality of polynucleotides differ by about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%. In some instances, the plurality of polynucleotides differ by about 1%, about 5%, about 10%, about 15%, about 20%, about 25%, or about 30%. In some instances, the plurality of polynucleotides differ by at least about 1%, about 5%, about 10%, about 15%, about 20%, or about 25%. In some instances, the plurality of polynucleotides differ by at most about 5%, about 10%, about 15%, about 20%, about 25%, or about 30%. In some instances, the efficiency of PCR is related to the amount of polynucleotides having the same length. In some instances, the plurality of polynucleotides having the same length ensures PCR does not change the distribution of the polynucleotides. In some instances, 90% or more the plurality of polynucleotides having the same length ensures PCR does not change the distribution of the polynucleotides.

In some instances, the number of synthesis cycles is less than 400 for a polynucleotide sequence comprising 100 bases. In some instances, the number of synthesis cycles is less than 300 for a polynucleotide sequence comprising 100 bases. In some instances, the number of synthesis cycles is less than 200 for a polynucleotide sequence comprising 100 bases. In some instances, the number of synthesis cycles is about 300 for a polynucleotide sequence comprising 100 bases. In some instances, the number of synthesis cycles is about 200 for a polynucleotide sequence comprising 200 bases. In some instances, the number of synthesis cycles is about 224 for a polynucleotide sequence comprising 100 bases. In some instances, the number of synthesis cycles is about 100 for a polynucleotide sequence comprising 100 bases. In some instances, the number of synthesis cycles is less than 800 for a polynucleotide sequence comprising about 200 bases. In some instances, the number of synthesis cycles is less than 600 for a polynucleotide sequence comprising 200 bases. In some instances, the number of synthesis cycles is less than 500 for a polynucleotide sequence comprising 200 bases. In some instances, the number of synthesis cycles is less than 400 for a polynucleotide sequence comprising 200 bases. In some instances, the number of synthesis cycles is less than 300 for a polynucleotide sequence comprising 200 bases. In some instances, the number of synthesis cycles is about 500 for a polynucleotide sequence comprising 200 bases. In some instances, the number of synthesis cycles is about 400 for a polynucleotide sequence comprising 200 bases. In some instances, the number of synthesis cycles is about 300 for a polynucleotide sequence comprising 200 bases. In some instances, the number of synthesis cycles is about 200 for a polynucleotide sequence comprising 200 bases. In some instances, the number of synthesis cycles is less than 1200 for a polynucleotide sequence comprising 300 bases. In some instances, the number of synthesis cycles is less than 1000 for a polynucleotide sequence comprising 300 bases. In some instances, the number of synthesis cycles is less than 800 for a polynucleotide sequence comprising 300 bases. In some instances, the number of synthesis cycles is less than 600 for a polynucleotide sequence comprising 300 bases. In some instances, the number of synthesis cycles is less than 400 for a polynucleotide sequence comprising 300 bases. In some instances, the number of synthesis cycles is about 600 for a polynucleotide sequence comprising 300 bases. In some instances, the number of synthesis cycles is about 500 for a polynucleotide sequence comprising 300 bases. In some instances, the number of synthesis cycles is about 450 for a polynucleotide sequence comprising 300 bases. In some instances, the number of synthesis cycles is about 450 for a polynucleotide sequence comprising 300 bases. In some instances, the polynucleotide sequence comprises four nucleotides. In some instances, the polynucleotide sequence comprises one or more of A, T, C, and G. In some instances, the polynucleotide sequence comprises one, two, three, four, or five nucleotides. In some instances, the polynucleotide sequence comprises at least one, two, three, four, or five nucleotides. In some instances, the polynucleotide sequence comprises at most one, two, three, four, or five nucleotides. In some instances, about 10%, 20%, 25%, 30%, 33%, 40%, 50%, 60%, 66%, 70%, 75%, 80%, or 90% of the polynucleotide sequence encodes for redundancy. In some instances, up to about 10%, 20%, 25%, 30%, 33%, 40%, 50%, 60%, 66%, 70%, 75%, 80%, or 90% of the polynucleotide sequence encodes for redundancy. In some instances, at most about 10%, 20%, 25%, 30%, 33%, 40%, 50%, 60%, 66%, 70%, 75%, 80%, or 90% of the polynucleotide sequence encodes for redundancy. In some examples, the polynucleotide sequence comprises about 1.5×, 2×, 2.5×, 3×, 3.5×, or 4× redundancy.

In some instances, the plurality of polynucleotides are synthesized on a solid support, such as those provided herein. The solid support can be a substrate as provided herein. In some instances, the solid support comprises a plurality of features (or loci). The plurality of polynucleotides can be synthesized on the plurality of features. In some instances, about 25% to about 80% of the plurality of features are deblocked per synthesis cycle. In some instances, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 40% to about 45%, about 40% to about 50%, about 40% to about 55%, about 40% to about 60%, about 40% to about 65%, about 40% to about 70%, about 40% to about 75%, about 40% to about 80%, about 45% to about 50%, about 45% to about 55%, about 45% to about 60%, about 45% to about 65%, about 45% to about 70%, about 45% to about 75%, about 45% to about 80%, about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or about 75% to about 80% of the plurality of features are deblocked per synthesis cycle. In some instances, about 25%, about 30%, about 35%, about 40%, about 45%, about 50%, about 55%, about 60%, about 65%, about 70%, about 75%, or about 80% of the plurality of features are deblocked per synthesis cycle. In some instances, at least about 25%, about 30%, about 35%, about 40%, about 45%, about 50%, about 55%, about 60%, about 65%, about 70%, or about 75% of the plurality of features are deblocked per synthesis cycle. In some instances, at most about 30%, about 35%, about 40%, about 45%, about 50%, about 55%, about 60%, about 65%, about 70%, about 75%, or about 80% of the plurality of features are deblocked per synthesis cycle.

The plurality of features on a solid support can be independently addressable. In some instances, the plurality of features are independently addressable by controlling access of reagents to certain sections. In some instances, the plurality of features are independently addressable by controlling reactivity of polynucleotides at each feature of the plurality of features. In some instances, the plurality of features are independently addressable through one or more electrodes of the solid-support. An example of a device comprising a solid support comprising an addressable locus (e.g., feature) is described in U.S. Pat. No. 10,936,953 or U.S. Pat. No. 9,267,213, which are incorporated herein by reference in its entirety. In some instances, the plurality of features are addressable through masking specific areas. In some examples, specific areas are chemically functionalized, such as, for example, by modifying the surface with hydrophobic or hydrophilic chemical groups. As an example, the plurality of features may be masked using methods described in U.S. Pat. Nos. 10,894,242, 10,195,580, or WO2022/047076, which are incorporated herein by reference in its entirety. In some instances, the plurality of features are addressable through electrochemical deblocking. In some instances, the plurality of features are addressable through acid-generation. In some instances, the one or more electrodes can be used to generate one or more chemical reactions (e.g., electrochemically generated acid (EGA) for nucleotide deprotection). In some instances, electrochemical deblocking comprises an organic solvent-based solution for deblocking during synthesis of any of a variety of oligomers, (e.g., oligonucleotides). In such cases, an acid-based chemical deblocking involves the removal of a blocking moiety on a molecule can allow for covalent binding of a next nucleotide. Electrochemical deblocking comprises application of a voltage or a current to one or more features via one or more electrodes on a solid support (e.g., an electrode microarray) to locally generate an acid or a base (depending on whether the electrode is an anode or a cathode), which can affect removal of acid- or base-labile protecting groups (moieties) bound to a chemical species. In some instances, masking techniques that are addressable using photogenerated acids are used in combination with photosensitizers for deblocking. In some instances, the plurality of features are addressable through metal-catalyzed deprotection (e.g., palladium-catalyzed deprotection).

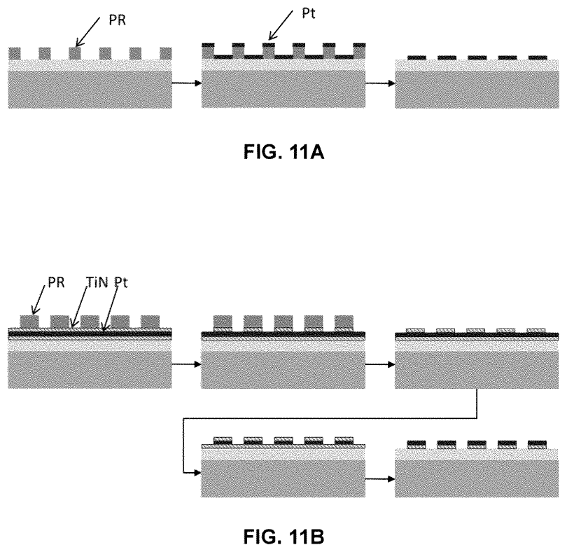

In some instances, the plurality of features can be addressable through masking methods. In some instances, a lift-off fabrication method can be used (FIG. 11A). Lift-off methods in some instances comprises addition of a sacrificial layer (e.g., photoresist or “PR”) to a base layer coated with an oxide layer, addition of a conductive layer, and removal of the sacrificial layer. In some instances, a dry-etch fabrication method can be used (FIG. 11B). Dry-etch methods in some instances comprises addition of one or more layers to a base layer, such as an oxide layer, a first intermediate layer (e.g., TiN, or other material), a conductive layer (e.g., platinum), a second intermediate layer (e.g., TiN, or other material), and a sacrificial layer (e.g., photoresist); partial removal of the second intermediate layer to expose the conductive layer; partial removal of the conductive layer to expose the first intermediate layer; partial removal of the first conductive layer to expose the first intermediate layer; and partial removal of the first intermediate layer to expose the oxide layer. As an example, a surface comprising a base layer of silicon and a top layer comprising an oxide can be patterned with a removable masking material, such as a photoresist (FIG. 11A). The entire surface including the mask can be plated with platinum, and the mask layer can then be removed. Previously masked regions are then exposed oxide, and unmasked regions comprise platinum on top of the oxide layer. As a further example, surface comprising a base layer of silicon, a first layer comprising an oxide, a second layer of titanium nitride, a third layer comprising platinum, a fourth layer comprising titanium nitride, (from bottom to top) can be patterned with a removable masking material, such as a photoresist (FIG. 11B). Unmasked fourth layer can be removed to expose the third layer, and the photoresist can be removed to expose the masked fourth layer. Removal of all remaining second and fourth layers can produce a surface comprising a base layer of silicon, and top layer of oxide, and “islands” of platinum patterned on top of titanium nitride.

In some instances, the one or more electrodes to generate an electrochemical reagent may comprise, by way of non-limiting example, metals such as iridium and/or platinum, and other metals, such as, palladium, gold, silver, copper, mercury, nickel, zinc, titanium, tungsten, aluminum, as well as alloys of various metals, and other conducting materials, such as, carbon, including glassy carbon, reticulated vitreous carbon, basal plane graphite, edge plane graphite or graphite. In some instances, doped oxides such as indium tin oxide, and semiconductors such as silicon oxide and gallium arsenide may also be used. Additionally, the electrodes may be composed of conducting polymers, metal doped polymers, conducting ceramics and conducting clays. In some instances, platinum and palladium comprise advantageous properties associated with their ability to absorb hydrogen (e.g., their ability to be “preloaded” with hydrogen before being used). In some instances, the one or more electrodes may be connected to an electric source. In some instances, the electrodes are connected to the electric source by way of CMOS (complementary metal oxide semiconductor) switching circuitry, radio and microwave frequency addressable switches, light addressable switches, direct connection from an electrode to a bond pad on the perimeter of a semiconductor chip, or any combination thereof. CMOS switching circuitry can comprise connection of each of the electrodes to a CMOS transistor switch. The switch may be accessed by sending an electronic address signal down a common bus to SRAM (static random access memory) circuitry associated with each electrode. When the switch is “on”, the electrode can be connected to an electric source. Radio and microwave frequency addressable switches can involve the electrodes being switched by a RF or microwave signal. This can allow the switches to be thrown both with and/or without using switching logic. The switches can be tuned to receive a particular frequency or modulation frequency and switch without switching logic. Light addressable switches may be switched by light. In some instances, the one or more electrodes can also be switched with and without switching logic. In some example, the light signal can be spatially localized to afford switching without switching logic, for example, by scanning a laser beam over the electrode array, where the electrode is switched each time a laser illuminates it.

Sequences of the plurality of polynucleotides may be determined. In some instances, the plurality of polynucleotides may be sequenced according to systems and methods provided herein. Sequencing may comprise, by way of non-limiting example, next generation sequencing, parallel sequencing, single-molecule real-time sequencing, nanopore sequencing, sequencing by synthesis, Sanger sequencing, or any combination thereof. In some instances, the plurality of polynucleotides may be sequenced via a sequencer. In some instances, sequencing the plurality of polynucleotides generates a plurality of output sequences. In some instances, the plurality of output sequences overlap with the plurality of polynucleotide sequences. In some instances, the overlap is about 50% to 100%. In some instances, the overlap is about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some instances, the overlap is about 50%, about 60%, about 70%, about 80%, about 90%, or about 100%. In some instances, the overlap is at least about 50%, about 60%, about 70%, about 80%, or about 90%. In some instances, the overlap is at most about 60%, about 70%, about 80%, about 90%, or about 100%. In some instances, the plurality of output sequences are decoded using methods described herein. For example, plurality of output sequences are decoded using a greedy algorithm, a maximum likelihood (ML) algorithm, or a mixed greedy ML algorithm. In some instances, plurality of output sequences are decoded based at least in part by calculating a probability of a deletion, insertion, mutation, or any combination thereof.

Further provided herein is a platform for encoding data. In some instances, the platform comprises a hybrid organic-in silico platform. In some instances, the platform comprises a computing system, a synthesizer, or a combination thereof. In some instances, a computing system comprising at least one processor and instructions executable by the at least one processor to perform operations. The computing system or the at least one processor may be those provided herein. In some instances, computing system comprises a distributed computing system. In some instances, the computing system comprises a cloud computing system. The cloud computing system can comprise a private cloud, a public cloud, a hybrid cloud, a multi-cloud, or any combination thereof. The cloud computing system can comprise an infrastructure as a service (IaaS), platform as a service (PaaS), software as a service (SaaS), or any combination thereof. In some instances, the operations comprise generating an inner codec comprising a codebook, such as those provided herein. In some instances, the codebook is optimized for one or more constraints, such as one or more constraints related to nucleic acid synthesis, post-processing, storage, or sequencing. In some instances, nucleic acid synthesis comprises electrochemical synthesis, enzymatic synthesis, phosphoramidite synthesis, inkjet printing, or any combination thereof. In some instances, the one or more constraints related to nucleic acid synthesis comprises a synthesis error, such as an insertion, deletion, or mutation. In some instances, post-processing comprises one or more of ligation, cleavage, hybridization, denaturation, fixation to a solid support, extension, error correction, enrichment, isolation, purification, and amplification. In some instances, storage comprises cold data storage. Cold data storage may generally refer to storage of data, for example, in nucleic acids, that is rarely accessed. Cold data storage may be the opposite of “hot storage” referring to data that is frequently accessed. In some examples, storage comprises hot storage, in which data stored in nucleic acids are frequently accessed. In some instances, storage comprises nucleic acid storage in a liquid phase or solid phase. In some examples, one or more constraints related to storage comprises temperature (e.g., room temperature), humidity, pressure, salinity, pH, concentration, time, light, UV, O2, or any combination thereof. In some instances, sequencing comprises next generation sequencing, parallel sequencing, single-molecule real-time sequencing, nanopore sequencing, sequencing by synthesis, Sanger sequencing, or any combination thereof.

In some instances, the codebook is generated with a base order (e.g., [A, T, C, G], etc.). In some instances, the codebook comprises codewords generated based on the base order. In some instances, the base order comprises predetermined base transitions. In some instances, the operations comprise applying the inner codec to encode the binary data as a plurality of polynucleotide sequences using methods provided herein.

In some instances, the synthesizer generates a plurality of polynucleotides comprising the plurality of polynucleotide sequences. In some instances, the synthesizer generates a plurality of polynucleotide sequences by synthesis, ligation, assembly, or any combination thereof. Methods of synthesis may be those provided herein (e.g., phosphoramidite, enzymatic, etc.). In some instances, the instructions from the computing system further cause the synthesizer to generate the plurality of polynucleotides. In some instances, the synthesizer is used for synthesis of polynucleotides. In some instances, the synthesizer is used for assembly of polynucleotides. In some instances, an alternative assembly module is used for assembly of the polynucleotides. In some examples, assembly comprises overlap-extension polymerase chain reaction (PCR), polymerase cycling assembly (PCA), sticky end ligation, biobricks assembly, golden gate assembly, gibson assembly, recombinase assembly, ligase cycling reaction, template directed ligation, or any combination thereof. In some instances, the synthesizer and the assembly module are in fluidic communication, electronic, communication, or a combination thereof.

The platform can further comprise a sequencer. The sequencer may comprise systems and devices for performing a sequencing method provided herein, or those known in the art. In some instances, the sequencer sequences the plurality of polynucleotides to generate a plurality of output sequences. Methods of sequencing may be those provided herein. In some instances, the instructions further cause the computing system to receive the plurality of output sequences. In some instances the computing system further performs operations comprising decoding the plurality of output sequences. The computing system can decode the plurality of output sequences or any other polynucleotide sequences using the decoding schemes provided herein. In some instances, the plurality of output sequences are decoded using a greedy algorithm, a maximum likelihood (ML) algorithm, or a mixed greedy ML algorithm. In some instances, the plurality of output sequences are decoded based at least in part by calculating a probability of a deletion, insertion, mutation, or any combination thereof. The platform can further comprise a storage unit. In some instances, the storage unit stores the plurality of polynucleotide. Polynucleotides may be stored in solution as a liquid, or dried as a solid. Polynucleotides may be stored on a substrate, such as those provided herein. In some instances, the instructions of the computing system cause the transfer of the plurality of polynucleotides between the synthesizer, the sequencer, the storage unit, or any combination thereof.

### De Novo Polynucleotide Synthesis

Provided herein are systems and methods for synthesis of polynucleotides on a substrate. In some instances, the final oligonucleotide pool from the inner codec is synthesized. In some instances, the library comprising a plurality of polynucleotides from the encoding scheme are synthesized 1225 (as shown in FIG. 12). In some examples, the library comprising the plurality of polynucleotides from the encoding scheme encode a pool of the plurality of pools. In some examples, the library comprising the plurality of polynucleotides from the encoding scheme encode an index pool. In some instances, methods comprise use of electrochemical deprotection. In some instances, the substrate is a flexible substrate. In some instances, at least 10¹⁰, 10¹¹, 10¹², 10¹³, 10¹⁴, or 10¹⁵ bases are synthesized in one day. In some instances, at least 10×10⁸, 10×10⁹, 10×10¹⁰, 10×10¹¹, or 10×10¹² polynucleotides are synthesized in one day. In some cases, each polynucleotide synthesized comprises at least 20, 50, 100, 200, 300, 400 or 500 nucleobases. In some cases, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized. In some instances, these at least 90%, 95%, 98%, 99%, 99.5%, or more of the polynucleotides synthesized do not differ from a predetermined sequence for which they encode. In some instances, the error rate for synthesized polynucleotides on a substrate using the methods and systems described herein is less than about 1 in 200, less than about 1 in 1,000, less than about 1 in 2,000, less than about 1 in 3,000, or less than about 1 in 5,000. Individual types of error rates include mismatches, deletions, insertions, and/or substitutions for the polynucleotides synthesized on the substrate. The term “error rate” refers to a comparison of the collective amount of synthesized polynucleotide to an aggregate of predetermined polynucleotide sequences. In some instances, the synthesis methods provided herein (e.g., inkjet based synthesis methods) have results better than about 0.1% deletion rate, 0.1% mutation rate (or substitution rate), 0.05% insertion rate, or any combination thereof. For example, the synthesized polynucleotide may have a deletion rate of less than or about 0.001%, 0.005%, 0.01%, 0.05%, 0.1%, a mutation rate of less than or about 0.001%, 0.005%, 0.01%, 0.05%, or 0.1%, an insertion rate of less than or about 0.001%, 0.005%, 0.01%, or 0.05%, or any combination thereof. In some instances, synthesized polynucleotides disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

Described herein are methods, systems, devices, and compositions wherein chemical reactions used in polynucleotide synthesis are controlled using electrochemistry. Electrochemical reactions in some instances are controlled by any source of energy, such as light, heat, radiation, or electricity. For example, electrodes are used to control chemical reactions as all or a portion of discrete loci on a surface. Electrodes in some instances are charged by applying an electrical potential to the electrode to control one or more chemical steps in polynucleotide synthesis. In some instances, these electrodes are addressable. Any number of the chemical steps described herein is in some instances controlled with one or more electrodes. Electrochemical reactions may comprise oxidations, reductions, acid/base chemistry, or other reaction that is controlled by an electrode. In some instances, electrodes generate electrons or protons that are used as reagents for chemical transformations. Electrodes in some instances directly generate a reagent such as an acid. In some instances, an acid is a proton. Electrodes in some instances directly generate a reagent such as a base. Acids or bases are often used to cleave protecting groups, or influence the kinetics of various polynucleotide synthesis reactions, for example by adjusting the pH of a reaction solution. Electrochemically controlled polynucleotide synthesis reactions in some instances comprise redox-active metals or other redox-active organic materials. In some instances, metal or organic catalysts are employed with these electrochemical reactions. In some instances, acids are generated from oxidation of quinones.

Control of chemical reactions with is not limited to the electrochemical generation of reagents; chemical reactivity may be influenced indirectly through biophysical changes to substrates or reagents through electric fields (or gradients) which are generated by electrodes. In some instances, substrates include but are not limited to nucleic acids. In some instances, electrical fields which repel or attract specific reagents or substrates towards or away from an electrode or surface are generated. Such fields in some instances are generated by application of an electrical potential to one or more electrodes. For example, negatively charged nucleic acids are repelled from negatively charged electrode surfaces. Such repulsions or attractions of polynucleotides or other reagents caused by local electric fields in some instances provides for movement of polynucleotides or other reagents in or out of region of the synthesis device or structure. In some instances, electrodes generate electric fields which repel polynucleotides away from a synthesis surface, structure, or device. In some instances, electrodes generate electric fields which attract polynucleotides towards a synthesis surface, structure, or device. In some instances, protons are repelled from a positively charged surface to limit contact of protons with substrates or portions thereof. In some instances, repulsion or attractive forces are used to allow or block entry of reagents or substrates to specific areas of the synthesis surface. In some instances, nucleoside monomers are prevented from contacting a polynucleotide chain by application of an electric field in the vicinity of one or both components. Such arrangements allow gating of specific reagents, which may obviate the need for protecting groups when the concentration or rate of contact between reagents and/or substrates is controlled. In some instances, unprotected nucleoside monomers are used for polynucleotide synthesis. Alternatively, application of the field in the vicinity of one or both components promotes contact of nucleoside monomers with a polynucleotide chain. Additionally, application of electric fields to a substrate can alter the substrates reactivity or conformation. In an exemplary application, electric fields generated by electrodes are used to prevent polynucleotides at adjacent loci from interacting. In some instances, the substrate is a polynucleotide, optionally attached to a surface. Application of an electric field in some instances alters the three-dimensional structure of a polynucleotide. Such alterations comprise folding or unfolding of various structures, such as helices, hairpins, loops, or other 3-dimensional nucleic acid structure. Such alterations are useful for manipulating nucleic acids inside of wells, channels, or other structures. In some instances, electric fields are applied to a nucleic acid substrate to prevent secondary structures. In some instances, electric fields obviate the need for linkers or attachment to a solid support during polynucleotide synthesis.

A suitable method for polynucleotide synthesis on a substrate of this disclosure is a phosphoramidite-based synthesis of DNA. In some cases, a reagent for the phosphoramidite-based synthesis comprises any one of or a combination of a nucleoside phosphoramidite, an oxidizer, an activator, or a deblocker or the solvent comprises acetonitrile. In some instances, the phosphoramidite-based synthesis method comprises the controlled addition of a phosphoramidite building block, i.e. nucleoside phosphoramidite, to a growing polynucleotide chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. Protecting groups may comprise any chemical group that prevents extension of the polynucleotide chain. In some instances, the protecting group is cleaved (or removed) in the presence of an acid. In some instances, the protecting group is cleaved in the presence of a base. In some instances, the protecting group is removed with electromagnetic radiation such as light, heat, or other energy source. In some instances, the protecting group is removed through an oxidation or reduction reaction. In some instances, a protecting group comprises a triarylmethyl group. In some instances, a protecting group comprises an aryl ether. In some instances, a protecting comprises a disulfide. In some instances a protecting group comprises an acid-labile silane. In some instances, a protecting group comprises an acetal. In some instances, a protecting group comprises a ketal. In some instances, a protecting group comprises an enol ether.

In some instances, a protecting group comprises a methoxybenzyl group. In some instances, a protecting group comprises an azide. In some instances, a protecting group is 4,4′-dimethoxytrityl (DMT). In some instances, a protecting group is a tert-butyl carbonate. In some instances, a protecting group is a tert-butyl ester. In some instances, a protecting group comprises a base-labile group.

Following coupling, phosphoramidite polynucleotide synthesis methods optionally comprise a capping step. In a capping step, the growing polynucleotide is treated with a capping agent. A capping step generally serves to block unreacted substrate-bound 5′—OH groups after coupling from further chain elongation, preventing the formation of polynucleotides with internal base deletions. Further, phosphoramidites activated with 1H-tetrazole often react, to a small extent, with the O6 position of guanosine. Without being bound by theory, upon oxidation with I2/water, this side product, possibly via O6-N7 migration, undergoes depurination. The apurinic sites can end up being cleaved in the course of the final deprotection of the polynucleotide thus reducing the yield of the full-length product. The O6 modifications may be removed by treatment with the capping reagent prior to oxidation with I2/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

Following addition of a nucleoside phosphoramidite, and optionally after capping and one or more wash steps, a substrate described herein comprises a bound growing nucleic acid that may be oxidized. The oxidation step comprises oxidizing the phosphite triester into a tetracoordinated phosphate triester, a protected precursor of the naturally occurring phosphate diester internucleoside linkage. In some instances, phosphite triesters are oxidized electrochemically. In some instances, oxidation of the growing polynucleotide is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

For a subsequent cycle of nucleoside incorporation to occur through coupling, a protected 5′ end (or 3′ end, if synthesis is conducted in a 5′ to 3′ direction) of the substrate bound growing polynucleotide is be removed so that the primary hydroxyl group can react with a next nucleoside phosphoramidite. In some instances, the protecting group is DMT and deblocking occurs with trichloroacetic acid in dichloromethane. In some instances, the protecting group is DMT and deblocking occurs with electrochemically generated protons. Conducting detritylation for an extended time or with stronger than recommended solutions of acids may lead to increased depurination of solid support-bound polynucleotide and thus reduces the yield of the desired full-length product. Methods and compositions described herein provide for controlled deblocking conditions limiting undesired depurination reactions. In some instances, the substrate bound polynucleotide is washed after deblocking. In some cases, efficient washing after deblocking contributes to synthesized polynucleotides having a low error rate.

Methods for the synthesis of polynucleotides on a substrate described herein may involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some instances, one or more wash steps precede or follow one or all of the steps.

Methods for the synthesis of polynucleotides on a substrate described herein may comprise an oxidation step. For example, methods involve an iterating sequence of the following steps: application of a protected monomer to a surface of a substrate feature to link with either the surface, a linker or with a previously deprotected monomer; deprotection of the applied monomer so that it can react with a subsequently applied protected monomer; application of another protected monomer for linking, and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

In some instances, polynucleotides are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of a substrate described herein that provides support for polynucleotide synthesis may be chemically modified to allow for the synthesized polynucleotide chain to be cleaved from the surface. In some instances, the polynucleotide chain is cleaved at the same time as the polynucleotide is deprotected. In some cases, the polynucleotide chain is cleaved after the polynucleotide is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the nucleic acid chain growth is supported. Cleavage includes gas cleavage with ammonia or methylamine. In some instances cleavage includes linker cleavage with electrically generated reagents such as acids or bases. In some instances, once released from the surface, polynucleotides are assembled into larger nucleic acids that are sequenced and decoded to extract stored information.

In some instances, synthesis comprises enzymatic synthesis. Enzymatic synthesis may be performed on a surface described herein. In some instances, enzymatic synthesis comprises a chain-elongating enzyme. In some instances, the chain-elongating enzyme is a polymerase. In some instances, the polymerase is a template-independent polymerase. In some instances, the polymerase is an RNA polymerase or DNA polymerase. In some instances, the polymerase is a DNA polymerase. In some cases, the enzymatic DNA synthesis uses water as a solvent and the reagent is an enzyme terminal deoxynucleotidyl transferase (TdT) or a deblocker. In some cases, enzymatic synthesis of DNA uses a template-independent DNA polymerase, terminal deoxynucleotidyl transferase (TdT), which is a protein that evolved to rapidly catalyze the linkage of naturally occurring dNTPs. TdT adds nucleotides indiscriminately so it is stopped from continuing unregulated synthesis by various techniques such a tethering the TDT, creating variant enzymes, and using nucleotides that include reversible terminators to prevent chain elongation. TdT activity is maximized at approximately 37° C. and performs enzymatic reactions in an aqueous environment. Examples of DNA polymerases include, but are not limited to, polA, polB, polC, polD, polY, polX, reverse transcriptases (RT), and high-fidelity polymerases. In some instances, the polymerase is a modified polymerase. In some embodiments, the polymerase comprises Φ29, B103, GA-1, PZA, Φ15, BS32, M2Y, Nf, G1, Cp-1, PRD1, PZE, SF5, Cp-5, Cp-7, PR4, PR5, PR722, L17, ThermoSequenase®, 9°Nm™, Therminator™ DNA polymerase, Tne, Tma, TfI, Tth, Tli, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1I/DP2 DNA polymerase II, 9°N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase. In some embodiments, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase. The polymerase molecules used in the methods described herein can be polymerase theta, a DNA polymerase, or any enzyme that can extend nucleotide chains. In some embodiments, the polymerase is tri29. In some embodiments, the polymerase is a protein with pockets that work around terminal phosphate groups, for example, a triphosphate group.

In some embodiments, enzymatic synthesis uses TdT with 1, 2, 3, 4, 5, 6, 7, 8, 9, or 10 amino acid mutations to synthesize defined polynucleotides. In some embodiments, the described method uses TdT with 1, 2, 3, 4, 5, 6, 7, 8, 9, or 10 amino acid mutations to a surface-accessible amino acid residue. In some embodiments, the TdT is a variant of TdT. In some embodiments, the variant of TdT comprises a cysteine mutation (e.g., NTT-1). In some embodiments, the variant of TdT is NTT-1, NTT-2, or NTT-3. In some instances, the variant TdT comprises at least 70%, 80%, 90%, or 95% sequence identity to wild-type TdT. In some embodiments, enzymatic synthesis can use polymerase theta with 1, 2, 3, 4, 5, 6, 7, 8, 9, or 10 amino acid mutations to synthesize defined polynucleotides. In some embodiments, enzymatic synthesis can use polymerase theta with 1, 2, 3, 4, 5, 6, 7, 8, 9, or 10 amino acid mutations to a surface-accessible amino acid residue. In some embodiments, the polymerase theta is a variant of polymerase theta. In some instances, the variant polymerase theta comprises at least 70%, 80%, 90%, or 95% sequence identity to wild-type polymerase theta. In some embodiments, the polymerase theta is encoded by POLQ.

Enzymes described herein (e.g., TdT), in some embodiments, comprise one or more unnatural amino acids. In some instances, the unnatural amino acid comprises: a lysine analogue; an aromatic side chain; an azido group; an alkyne group; or an aldehyde or ketone group. In some instances, the unnatural amino acid does not comprise an aromatic side chain. In some embodiments, the unnatural amino acid is selected from N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine (pAzF), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl) alanine, 2-amino-3-((2-((3-(benzyloxy)-3-oxopropyl)amino)ethyl) selanyl) propanoic acid, 2-amino-3-(phenylselanyl) propanoic, selenocysteine, N6-(((2-azidobenzyl)oxy) carbonyl)-L-lysine, N6-(((3-azidobenzyl)oxy) carbonyl)-L-lysine, and N6-(((4-azidobenzyl)oxy) carbonyl)-L-lysine. In some embodiments, the enzymes described herein are fused to one more other enzymes. For example, TdT is fused to other enzymes such as helicase.

Various linkers may be used for conjugating an enzyme or other nucleic acid (e.g., polymerase) binding moiety to one or more base-pairing moieties, e.g., a modified nucleotide during enzymatic synthesis of the polynucleotides. Conjugation of nucleotides or other base-pairing moieties to linkers may be achieved by any means known in the art of chemical conjugation methods. For example, nucleotides containing base modifications that add a free amine group are contemplated for use in conjugation to linkers as described herein. Primary amines, for example, may be linked to the base in such a manner that they can be reacted with heterobifunctional polyethylene glycol (PEG) linkers to create a nucleotide containing a variable length PEG linker that will still bind properly to the enzyme active site. Examples of such amine-containing nucleotides include 5-propargylamino-dNTPs, 5-propargylamino-NTPs, amino allyl-dNTPs, and amino allyl-NTPs. In some embodiments, amine-containing nucleotides are suitable for conjugation with PEG-based linkers. PEG linkers may vary in length, for example, from 1-1000, from 1-500, from 1-11, from 1-100, from 1-50, or from 1-10 subunits. Non-limiting examples of other suitable linkers may comprise, but are not limited to, poly-T and poly-A oligonucleotide strands (e.g., ranging from about 1 base to about 1,000 bases in length), peptide linkers (e.g., poly-glycine or poly-alanine ranging from about 1 residue to about 1,000 residues in length), or carbon-chain linkers (e.g., C6, C12, C18, C24, etc.). In some embodiments, the linker contains an N-hydroxysuccinimide ester (NHS) group. In some embodiments, the linker contains a maleimide group. Connection of the nucleotide can be achieved by the formation of a disulfide (forming a readily cleavable connection), formation of an amide, formation of an ester, protein-ligand linkage (e.g., biotin-streptavidin linkage), by alkylation (e.g., using a substituted iodoacetamide reagent) or forming adducts using aldehydes and amines or hydrazines. In some embodiments, the linker contains, e.g., a maltose group, a biotin group, an O2-benzylcytosine group or O2-benzylcytosine derivative, an O6-benzylguanine group, or an O6-benzylguanine derivative. The length of the linker may vary depending on the type of nucleotide (or other base-pairing moiety) and the enzyme (or other nucleic acid binding moiety). In some instances, a linker for connecting the nucleotide to the enzyme can have a persistence length of about 0.1-1,000 nm, 0.5-500 nm, 0.5-400 nm, 0.5-300 nm, 0.5-200 nm, 0.5-100 nm, 0.5-50 nm, 0.6-500 nm, 0.6-400 nm, 0.6-300 nm, 0.6-200 nm, 0.6-100 nm, 0.6-50 nm, 1-500 nm, 1-400 nm, 1-300 nm, 1-200 nm, 1-100 nm, 1.5-500 nm, 1.5-400 nm, 1.5-300 nm, 1.5-200 nm, 1.5-100 nm, 1.5-50 nm, 1-50 nm, 5-500 nm, 5-400 nm, 5-300 nm, 5-200 nm, 5-100 nm, or 5-50 nm. In some embodiments, the chemical linker is an acid-cleavable linker. In some embodiments, the chemical linker is an acid-cleavable linker. In some embodiments, the chemical linker is a photo-cleavable linker. In some embodiments, the chemical linker is selected from the group consisting of a silyl linker, an alkyl linker, a polyether linker, a polysulfonyl linker, a polysulfoxide linker, and any combination thereof. In some embodiments, the linker is cleaved by an enzyme. In some embodiments, the enzyme is a protease, an esterase, a glycosylase, or a peptidase. In some embodiments, the cleaving enzyme breaks bonds in the polymerase. In some embodiments, the cleaving enzyme directly cleaves the linked nucleoside.

The surfaces described herein can be reused after polynucleotide cleavage to support additional cycles of polynucleotide synthesis. For example, the linker can be reused without additional treatment/chemical modifications. In some instances, a linker is non-covalently bound to a substrate surface or a polynucleotide. In some embodiments, the linker remains attached to the polynucleotide after cleavage from the surface. Linkers in some embodiments comprise reversible covalent bonds such as esters, amides, ketals, beta substituted ketones, heterocycles, or other group that is capable of being reversibly cleaved. Such reversible cleavage reactions are in some instances controlled through the addition or removal of reagents, or by electrochemical processes controlled by electrodes. Optionally, chemical linkers or surface-bound chemical groups are regenerated after a number of cycles, to restore reactivity and remove unwanted side product formation on such linkers or surface-bound chemical groups.

### Devices for Polynucleotide Storage

The synthesized libraries of polynucleotides can be stored in a device. In some cases, the device comprises a polynucleotide data storage system. In some cases, the libraries encoding pools (e.g., a plurality of pools or index pools) are stored in compartments. In some instances, the compartments comprise, by way of non-limiting example, active surfaces (e.g., loci), tubes, or any other physical storage solutions. In some examples, the compartments are marked with a label. In some examples, the label comprises a barcode, a name (e.g., customer name, sample type, etc.), a timestamp, a list of objects stored, or any combination thereof.

In some cases, the device for storing digital information in DNA comprises one or more compartments. In some instances, each of the one or more compartments comprises a library comprising a plurality of polynucleotides. In some examples, the library encodes a pool comprising digital information corresponding to one or more objects (e.g., a pool of the plurality of pools described herein). In some examples, the pool comprises a pool descriptor, one or more pool items, an end pool descriptor, such as those described herein. In some examples, the pool comprises about 1 GB to about 1 TB of digital information, as previously described herein.

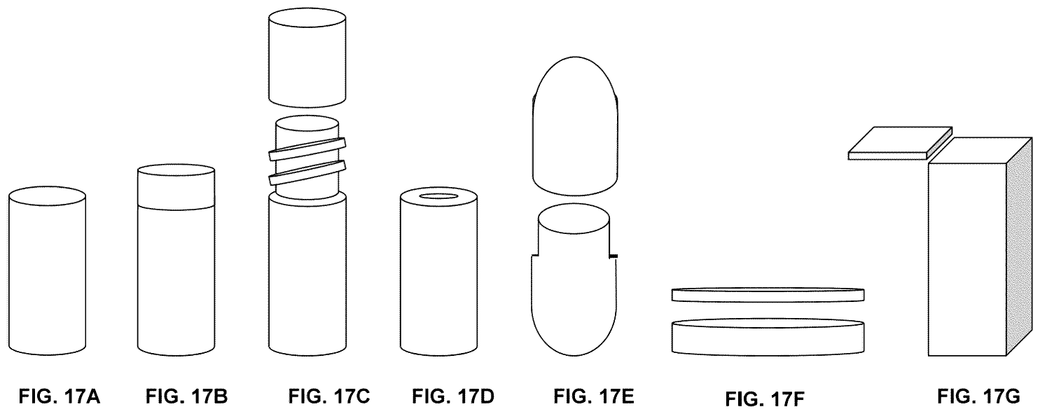

A compartment or structure for storing the plurality of polynucleotides may be any shape or size. In some instances, the compartment is substantially spherical, tubular (FIG. 17A), egg-shaped, conical, cubic, cuboid, cylindrical, wedge, hexagonal prism, square base pyramid, triangular based pyramid, triangular prism, toroid, hemisphere, helical, heart-shaped, or other shape. In some instances, shapes are configured to allow the structure to be opened or closed to the outside environment. In some instances, such closures are faciliated by welding, seals, septums, or other mechanism for restricting the movement of gases or other matter in or out of the structure. In some instances, the compartment comprises holes, slots, septum, valves, or ports for addition or removal of nucleic acids, fluids, gases, or other material into or out of the structure. In some instances a structure for storing the plurality of polynucleotides comprises a cap and a body that are flush-welded together (FIG. 17B). In some instances, a compartment for storing the plurality of polynucleotides comprises a removable screw-cap (FIG. 17C). In some instances, a structure comprises a septum (FIG. 17D). In some instances a structure comprises two rounded, pill-shaped halves that form a seal when one half is inserted into the other (FIG. 17E). In some instances, a structure comprises a substantially flat, disc container with sealable lid (FIG. 17F). In some intances, a compartment comprises a box with an optionally attached lid (FIG. 17G). In some examples, the shape is a cylinder or a disk. In some examples, a cylinder or a disk shape is preferrable for automated handling and/or filing of the compartments.

In some instances, each of the one or more compartments comprises a medium for storing the plurality of polynucleotides. In some examples, the medium comprises a solid, a liquid, a gas, or any combination thereof. In some examples, the medium comprises a salt solution. In some examples, the molar ratio of salt to DNA may range from about 20:1 to about 2:1. In some examples, the molar ratio depends on the molecular weight of the salt used and on the relative amounts of salt and DNA combined. In some examples, the molar ratio is calculated between the cation of the salt and the negatively charged phosphate groups of the DNA. In some examples, the salt solution comprises a molar ratio of less than 20:1 salt cation to phosphate groups in the DNA. In some examples, the salt solution is dried to create a dried product. In some cases, the salt solution comprises, by way of non-limiting examples, calcium chloride, calcium nitrate, calcium carbonate, calcium phosphate, magnesium chloride, magnesium sulfate, magnesium nitrate, magnesium carbonate, lanthanum chloride, lanthanum nitrate, lanthanum carbonate, lanthanum bromide, or a mixture thereof. In some instances, the salt solution comprises barium (II) chloride dihydrate, calcium chloride dihydrate, copper (II) chloride anhydrous, lanthanum trichloride, magnesium dichloride hexahydrate, sodium chloride, or strontium chloride hexahydrate. In some instances, the concentration of the salt solution is about 0.01 nM to about 0.1 nM.

In some cases, each of the one or more compartments are in communication. In some instances, each of the one or more compartments are in communication through the medium. In some cases, each of the one or more compartments are not in communication. In some instances, each of the one or more compartments are not in communication through the medium.

In some cases, the device further comprises one or more second compartments. In some instances, each of the one or more second compartments comprises a second library. In some examples, the second library encodes an index pool, such as those described herein. In some cases, the one or more second compartments comprise a medium as previously described herein. In some cases, the one or more second compartments comprise the same medium as the one or more compartments. In some cases, the one or more second compartments comprise different media as the one or more compartments. In some cases, each of the one or more second compartments are in communication with each other and/or the one or more compartments (e.g., through the medium). In some cases, each of the one or more second compartments are not in communication with each other and/or the one or more compartments.

In some cases, the device further comprises a solid support comprising a surface. A such. described herein are devices for solid support based nucleic acid synthesis and storage, wherein the solid support has varying dimensions. In some instances, a size of the solid support is between about 40 and 120 mm by between about 25 and 100 mm. In some instances, a size of the solid support is about 80 mm by about 50 mm. In some instances, a width of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, a height of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, the solid support has a planar surface area of at least or about 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness of the solid support is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness of the solid support include 275 mm, 375 mm, 525 mm, 625 mm, 675 mm, 725 mm, 775 mm and 925 mm. In some instances, the thickness of the solid support is at least or about 0.5 mm, 1.0 mm, 1.5 mm, 2.0 mm, 2.5 mm, 3.0 mm, 3.5 mm, 4.0 mm, or more than 4.0 mm.

Described herein are devices wherein two or more solid supports are assembled. In some instances, solid supports are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports. In some instances, two or more solid supports are not interfaced with each other. Nucleic acids (and the information stored in them) present on solid supports can be accessed from the rack unit. Access includes removal of polynucleotides from solid supports, direct analysis of polynucleotides on the solid support, or any other method which allows the information stored in the nucleic acids to be manipulated or identified. Information in some instances is accessed from a plurality of racks, a single rack, a single solid support in a rack, a portion of the solid support, or a single locus on a solid support. In various instances, access comprises interfacing nucleic acids with additional devices such as mass spectrometers, HPLC, sequencing instruments, PCR thermocyclers, or other device for manipulating nucleic acids. Access to nucleic acid information in some instances is achieved by cleavage of polynucleotides from all or a portion of a solid support. Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, access to nucleic acid information (including removal/addition of racks, solid supports, reagents, nucleic acids, or other component) is completely automated.

Solid supports as described herein comprise an active area. In some instances, the active area comprises regions or loci for nucleic acid synthesis. In some instances, the active area comprises regions or loci for nucleic acid storage. In some examples, the regions or loci comprise the one or more compartments. In some examples, the regions or loci comprise the second one or more compartments. In some instances, the regions are addressable. In some examples, the regions are addressable through an electrode.

The active area comprises varying dimensions. For example, the dimension of the active area is between about 1 mm to about 50 mm by about 1 mm to about 50 mm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 mm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 mm.

Described herein are devices for solid support based nucleic acid synthesis and storage, wherein the solid support has a number of sites (e.g., spots) or positions for synthesis or storage. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances the area is up to 0.25, 0.5, 0.75, 1.0, 1.25, 1.5, or 2.0 inches squared. In some instances, the solid support comprises loci having a pitch of at least or about 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 μm. In some instances, the solid support comprises loci having a pitch of about 5 μm. In some instances, the solid support comprises loci having a pitch of about 2 μm. In some instances, the solid support comprises loci having a pitch of about 1 μm. In some instances, the solid support comprises loci having a pitch of about 0.2 μm. In some instances, the solid support comprises loci having a pitch of about 0.2 μm to about 10 μm, about 0.2 to about 8 μm, about 0.5 to about 10 μm, about 1 μm to about 10 μm, about 2 μm to about 8 μm, about 3 μm to about 5 μm, about 1 μm to about 3 μm or about 0.5 μm to about 3 μm. In some instances, the solid support comprises loci having a pitch of about 0.1 μm to about 3 um.

The solid support for nucleic acid synthesis or storage as described herein comprises a high capacity for storage of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 megabytes. In some instances, the capacity of the solid support is between about 1 to 10, 1 to 50, 1 to 100, 1 to 500, 1 to 1000, 10 to 50, 10 to 100, 10 to 500, 10 to 1000, 50 to 100, 50 to 500, 50 to 1000, 100 to 500, 100 to 1000, 200 to 500, 200 to 1000, 500 to 1000, or between about 800 to 1000 megabytes. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 gigabytes. In some instances, the capacity of the solid support is between about 1 to 10, 1 to 50, 1 to 100, 1 to 500, 1 to 1000, 10 to 50, 10 to 100, 10 to 500, 10 to 1000, 50 to 100, 50 to 500, 50 to 1000, 100 to 500, 100 to 1000, 200 to 500, 200 to 1000, 500 to 1000, or between about 800 to 1000 gigabytes. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 terabytes. In some instances, the capacity of the solid support is between about 1 to 10, 1 to 50, 1 to 100, 1 to 500, 1 to 1000, 10 to 50, 10 to 100, 10 to 500, 10 to 1000, 50 to 100, 50 to 500, 50 to 1000, 100 to 500, 100 to 1000, 200 to 500, 200 to 1000, 500 to 1000, or between about 800 to 1000 terabytes. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to 10, 1 to 50, 1 to 100, 1 to 500, 1 to 1000, 10 to 50, 10 to 100, 10 to 500, 10 to 1000, 50 to 100, 50 to 500, 50 to 1000, 100 to 500, 100 to 1000, 200 to 500, 200 to 1000, 500 to 1000, or between about 800 to 1000 petabytes. In some instances, the capacity of the solid support is about 100 petabytes.

In some instances, the data is stored as arrays of packets as droplets. In some examples, the arrays of packets are addressable packets. In some examples, the packets are addressable using an electrode. In some instances, the data is stored as arrays of packets as droplets on a spot. In some instances, the data is stored as arrays of packets as dry wells. In some instances, the arrays comprise at least or about 1, 2, 3, 4, 5, 6, 7, 8, 9, 10, 20, 50, 100, 200, or more than 200 gigabytes of data. In some instances, the arrays comprise at least or about 1, 2, 3, 4, 5, 6, 7, 8, 9, 10, 20, 50, 100, 200, or more than 200 terabytes of data. In some instances, an item of information is stored in a background of data. For example, an item of information encodes for about 10 to about 100 megabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 megabytes of data and is stored in 1, 10, 20, 30, 40, 50, 60, 70, 80, 90, 100, 150, 200, 300, 400, 500, or more than 500 petabytes of background data.

Provided herein are devices for solid support based nucleic acid synthesis and storage, wherein following synthesis, the polynucleotides are collected in packets as one or more droplets. In some instances, the polynucleotides are collected in packets as one or more droplets and stored. In some instances, a number of droplets is at least or about 1, 10, 20, 50, 100, 200, 300, 500, 1000, 2500, 5000, 75000, 10,000, 25,000, 50,000, 75,000, 100,000, 1 million, 5 million, 10 million, 25 million, 50 million, 75 million, 100 million, 250 million, 500 million, 750 million, or more than 750 million droplets. In some instances, a droplet volume comprises 5, 10, 15, 20, 25, 30, 35, 40, 45, 50, 55, 60, 65, 70, 75, 80, 85, 90, 95, 100, or more than 100 um (micrometer) in diameter. In some instances, a droplet volume comprises 1-100 μm, 10-90 um, 20-80 um, 30-70 μm, or 40-50 um in diameter.

In some instances, the polynucleotides that are collected in the packets comprise a similar sequence. In some instances, the polynucleotides further comprise a non-identical sequence to be used as a tag or barcode. For example, the non-identical sequence is used to index the polynucleotides stored on the solid support and to later search for specific polynucleotides based on the non-identical sequence. Exemplary tag or barcode lengths include barcode sequences comprising, without limitation, about 1, 2, 3, 4, 5, 6, 7, 8, 9, 10, 15, 20, 25 or more bases in length. In some instances, the tag or barcode comprise at least or about 10, 50, 75, 100, 200, 300, 400, or more than 400 base pairs in length.

Provided herein are devices for solid support based nucleic acid synthesis and storage, wherein the polynucleotides are collected in packets comprising redundancy. For example, the packets comprise about 100 to about 1000 copies of each polynucleotide. In some instances, the packets comprise at least or about 50, 75, 100, 200, 300, 400, 500, 600, 700, 800, 900, 1000, 1200, 1400, 1600, 1800, 2000, or more than 2000 copies of each polynucleotide. In some instances, the packets comprise about 1000X to about 5000X synthesis redundancy. Synthesis redundancy in some instances is at least or about 500X, 1000X, 1500X, 2000X, 2500X, 3000X, 3500X, 4000X, 5000X, 6000X, 7000X, 8000X, or more than 8000X. The polynucleotides that are synthesized using solid support based methods as described herein comprise various lengths. In some instances, the polynucleotides are synthesized and further stored on the solid support. In some instances, the polynucleotide length is in between about 100 to about 1000 bases. In some instances, the polynucleotides comprise at least or about 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more than 2000 bases in length. Sequencing

Polynucleotides are extracted and/or amplified from surfaces where they are synthesized or stored. After extraction and/or amplification of polynucleotides from the surface of a structure, suitable sequencing technology may be employed to sequence the polynucleotides. In some cases, the DNA sequence is read on the substrate or within a feature of a structure. In some cases, the polynucleotides stored on the substrate are extracted, and optionally assembled into longer nucleic acids and then sequenced. The polynucleotides may be extracted from the substrate using systems and methods described herein.

Polynucleotides synthesized and stored on the structures described herein encode data that can be retrieved or interpreted by reading the sequence of the synthesized polynucleotides and converting the sequence into a representation (e.g., string of symbols such as binary code) readable by a computer. In some cases the sequences require assembly, and the assembly step may need to be at the polynucleotide sequence stage or at the digital sequence stage.

Provided herein are detection systems comprising a device capable of sequencing stored polynucleotides, either directly on the structure and/or after removal from the main structure (e.g., synthesis structure, storage structure, etc.). In cases where the structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a polynucleotide (e.g., a fluorescent signal). In some instances, information encoded within polynucleotides on a continuous tape is read by a computer as the tape is conveyed continuously through a detector operably connected to the computer. In some instances, a detection system comprises a computer system comprising a polynucleotide sequencing device, a database for storage and retrieval of data relating to polynucleotide sequence, software for converting DNA code of a polynucleotide sequence to a string of symbols, such as binary code, a computer for reading the binary code, or any combination thereof.

Provided herein are sequencing systems that can be integrated into the devices described herein. Various methods of sequencing are well known in the art, and comprise “base calling” wherein the identity of a base in the target polynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents/voltages generated by polymerase-catalyzed addition of bases to a template strand. In some instances, synthesis surfaces comprise enzymes, such as polymerases. In some instances, such enzymes are tethered to electrodes or to the synthesis surface. In some instances, enzymes comprise terminal deoxynucleotidyl transferases, or variants thereof.

In some instances, the polynucleotides cleaved from a substrate surface or the amplified polynucleotides can be processed by techniques such as conventional or massively parallel sequencing. The sequencing can be done via various methods available in the field, e.g., methods involving incorporating one or more chain-terminating nucleotides, e.g., Sanger Sequencing method that can be performed by, e.g., SeqStudio® Genetic Analyzer from Applied Biosystems. In other embodiments, the sequencing can include performing a Next Generation Sequencing (NGS) method, e.g., primer extension followed by semiconductor-based detection (e.g., Ion Torrent™ systems from Thermo Fisher Scientific) or via fluorescent detection (e.g., Illumina systems).

### Methods and Systems for Information Retrieval

Provided herein are methods and systems for retrieving information (e.g., digital information). In some instances, provided herein are methods and systems for decoding. In some cases, the methods and systems decode polynucleotide sequences (e.g., polynucleotides, oligonucleotides, plurality of polynucleotides, etc.). In some instances, the polynucleotide sequences are encoded using the methods described herein. In some instances, the methods and systems comprise an inner codec, an outer codec, or a combination thereof. In some cases, the information comprises one or more objects, as previously described herein. In some cases, each of the one or more objects is about 1 GB to about 1 TB, as previously described herein. In some cases, the one or more objects comprises an item of information, such as, but not limited to, those described herein.

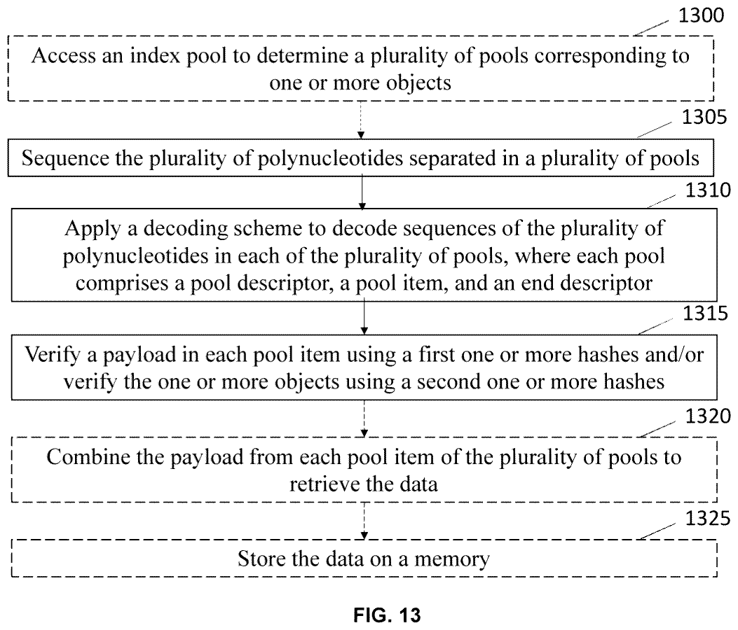

In some cases, the systems and methods decode polynucleotide sequences (e.g., polynucleotides, oligonucleotides, plurality of polynucleotides, etc.). An exemplary method for retrieving a digital information stored in a plurality of polynucleotides is illustrated in FIG. 13. In such instances, the plurality of polynucleotides may have been split into a plurality of pools following the general operations illustrated in FIG. 12. In some cases, a method for retrieving a digital information stored in a plurality of polynucleotides comprises one or more operations illustrated in FIG. 13.

In some cases, retrieving a digital information stored in a plurality of polynucleotides comprises accessing an index pool 1300. In some instances, accessing an index pool comprises fully or partially sequencing a library encoding an index pool. In some examples, the index pool is encoded in the library using the systems and methods described herein. In some examples, the polynucleotides in a library encoding an index pool are sequenced using the systems and methods described herein. In some instances, more than one index pool are accessed. In some instances, the polynucleotides in more than one library are sequenced. In some instances, the sequenced library is temporarily stored in a memory storage system (e.g. flash drives). In some instances, the sequenced library is converted to digital information to retrieve an index pool. In some instances, the index pool is temporarily stored in a memory storage system (e.g. flash drives). In some instances, the digital information in the index pool is used to search for one or more objects of interest. In some examples, the one or more objects of interest are stored in a library comprising a plurality of polynucleotides encoding the one or more objects. In some examples, the one of more objects of interest are searched using a metadata associated with the one or more object. In some instances, accessing an index pool determines a plurality of pools corresponding to one or more objects. However, in some instances, the one or more objects in one or more pools of the plurality of pools may be known, and access to an index pool may not be needed.

In some cases, the one or more objects of interest is retrieved, for example, from a compartment in a storage device. In some cases, retrieving a digital information stored in a plurality of polynucleotides comprises sequencing the plurality of polynucleotides corresponding to one or more objects in a plurality of pools 1305. In some instances, the plurality of polynucleotides are in a library. In some instances, the library is in a compartment of a device, as previously described herein. In some instances, the plurality of polynucleotides in a library encoding a pool are sequenced using the systems and methods described herein. In some cases, the pool is encoded in the library using the systems and methods described herein. In some instances, the plurality of polynucleotides in more than one compartment is sequenced to retrieve the one or more objects.

In some cases, retrieving a digital information stored in a plurality of polynucleotides further comprises applying a decoding scheme 210. In some instances, the decoding scheme decodes the digital information in the plurality of pools. In some instances, the decoding scheme is applied to the sequenced library comprising a plurality of polynucleotides. In some instances, a decoding scheme comprises an inner codec, an ECC, or a combination thereof. In some instances, the decoding scheme decodes a plurality of polynucleotide sequences to generate an output comprising digital information (e.g., an object). In some instances, the decoding scheme comprises undoing operations in the encoding scheme. In some examples, the operations comprise, splitting, shuffling, concatenating, transposing, translating, duplicating, labeling (e.g., using an index) data or a part of the data, or any combination thereof.

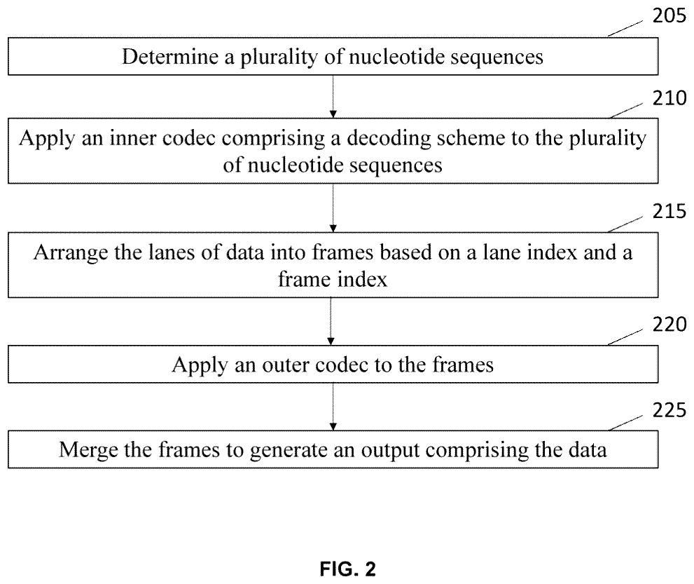

A method of decoding a plurality of polynucleotide sequences to generate an output comprising data (e.g., binary data) is schematically illustrated for example in FIG. 2. In some instances, methods for decoding the plurality of polynucleotide sequences may comprises determining the plurality of polynucleotide sequences 205. In some cases, determining the plurality of polynucleotide sequences comprises sequencing the nucleotides. In some instances, the nucleotides are sequenced using the methods described herein.

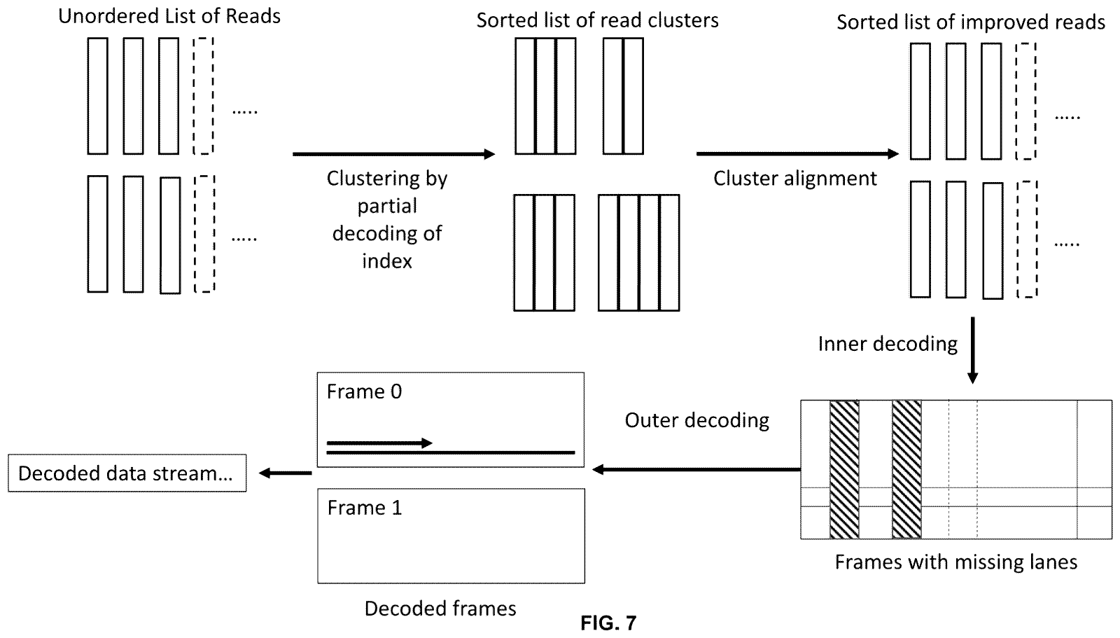

After sequencing the plurality of nucleotides, the encoded data (e.g., one or more objects) is decoded. In some instances, the plurality of nucleotides are decoded using the schematic illustrated, by way of non-limiting example, in FIG. 7. The output from sequencing comprises an unordered list of reads (e.g., polynucleotide sequences), as shown in FIG. 7.

In some instances, the sequenced and/or unordered reads are clustered after sequencing. In some cases, clustering is performed prior to applying the inner codec. In some instances, the reads are clustered based on an index, such as the frame index, the lane index, or a combination thereof. In such instances, the reads are partially decoded to obtain the frame index, the lane index, or the combination thereof. In some instances, clustering is performed using a hash function, as previously described herein. In some instances, a hash function is used if the bases in the polynucleotide sequences were determined using a hash in the encoding scheme, as previously described herein.

In some instances, the sequenced reads are aligned. In some instances, the sequenced polynucleotides are aligned after they have been clustered. In some instances, the clustered reads are aligned. In some cases, the reads are aligned prior to applying the inner codec. In some instances, aligning comprises analyzing consensus of the reads (e.g., nucleic acid or polynucleotide sequences) using an alignment algorithm. In some examples, the alignment algorithm comprises a pairwise alignment algorithm, a multi-sequence alignment algorithm, or a combination thereof.

In some instances, a pairwise alignment algorithm comprises initializing a position for each read. Initializing comprises aligning a polynucleotide sequence to a position 0. Consensus of a next one or more bases are analyzed between reads. In some instances, about 3 to about 10 reads are analyzed for consensus. In some instances, about 3 to about 4, about 3 to about 5, about 3 to about 6, about 3 to about 7, about 3 to about 8, about 3 to about 9, about 3 to about 10, about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10, or about 9 to about 10 reads are analyzed for consensus. In some instances, about 3, about 4, about 5, about 6, about 7, about 8, about 9, or about 10 reads are analyzed for consensus. In some instances, at least about 3, about 4, about 5, about 6, about 7, about 8, or about 9 reads are analyzed for consensus. In some instances, at most about 4, about 5, about 6, about 7, about 8, about 9, or about 10 reads are analyzed for consensus. In some instances, the next one or more bases comprise the next 2 to 10 bases. In some instances, the next one or more bases is about 2, 3, 4, 5, 6, 7, 8, 9, or 10 bases. In some instances, the next one or more bases is at least about 2, 3, 4, 5, 6, 7, 8, or 9 bases. In some instances, the next one or more bases is at most about 3, 4, 5, 6, 7, 8, 9, or 10 bases. In some instances, the next one or more bases is about 2, 3, 4, or 5 bases. The consensus is analyzed between the reads, and it is determined whether the next one or more bases are correct. If there is consensus between a base at a position, e.g., x, between all reads, then the subsequent base, e.g., x+1, may then be analyzed. If there is an inconsistencies in a base at a position, e.g., x, among the reads, then it is determined whether the read comprising the inconsistency has an error. In some instances, the error is an insertion, deletion, or substitution. The position is then incremented, e.g., x+1, given the decision (e.g., whether it is correct or has an error) for each read. In some instances, the steps are repeated until the end of a read is reached.

In some instances, the methods for decoding a plurality of polynucleotide sequences (or decoding schemes) comprise an inner codec. In some instances, the inner codec is applied to the plurality of nucleic acid (or polynucleotides) sequences. In some instances, the inner codec comprises a decoding scheme. The inner codec is used to transform the polynucleotide sequences into data (e.g., digital or binary data). In some instances, the inner codec is capable of correcting deletion, substitution, or insertion errors, or any combination thereof. For example, the inner codec provided herein may correct for errors up to 12% deletions, 6% mutations, or 2% insertions, or any combination thereof. In some examples, the inner codec can correct for errors up to 6% deletions, 3% mutations, or 1% insertions, or any combination thereof. In some examples, the inner codec can correct for errors of about 1%, 2%, 3%, 4%, 5%, 6%, 7%, 8%, 9%, 10%, 11%, or 12% deletions; about 1%, 2%, 3%, 4%, 5%, or 6% mutations; or about 1% or 2% insertions; or any combination thereof. In some examples, the inner codec can correct for errors of at most about 1%, 2%, 3%, 4%, 5%, 6%, 7%, 8%, 9%, 10%, 11%, or 12% deletions; about 1%, 2%, 3%, 4%, 5%, or 6% mutations; or about 1% or 2% insertions; or any combination thereof. In some further embodiments, the inner codec is used to validate oligos and discard any suspicious oligos to avoid contaminating the outer decoding. In some instances, the inner codec allows for efficient decoding using the indices (frame index and lane index).

An inner codec comprising a decoding scheme is applied to the plurality of polynucleotide sequences 210. The decoding scheme of the inner codec may transform each of the plurality of polynucleotide sequences into lanes of data. In some instances, the inner codec is applied to a plurality of nucleotides that have been sequenced. In some instances, the inner codec is applied to the unordered reads. In some instances, the inner codec is applied to the reads or the plurality of nucleotides once they have been clustered, as described herein. In some instances, the inner codec is applied to the reads or the plurality of nucleotides once they have been aligned, as described herein.

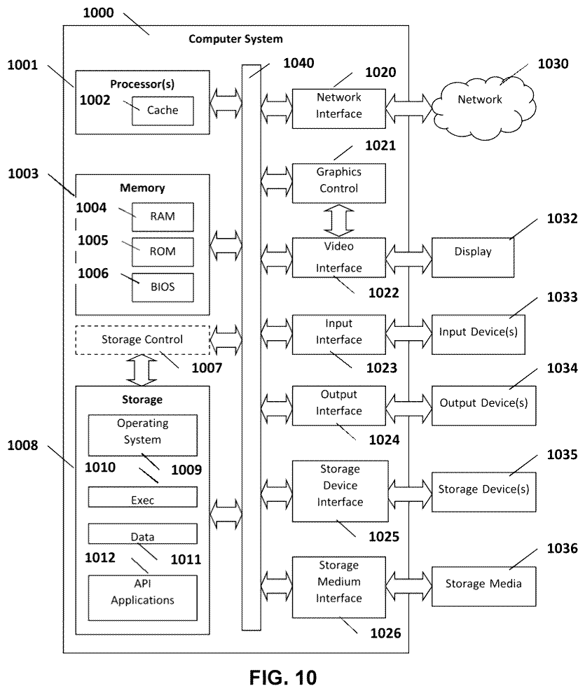

In some instances, the decoding scheme may decode the reads at a rate of at least about 50,000, 100,000, 150,000, or 200,000 reads per second provided that the software is running on an 8 core processing chip, for example, 8-core Intel® 9i. In some examples, the decoding scheme decodes the reads at a rate of at least about 100,000 reads per second (e.g., about 0.5 billion reads per hour corresponding to about 138,000 reads per second) provided that the software is running on an 8 core processing chip, (e.g., 8-core Intel® 9i). However, one of skill in the art will appreciate that the rate of decoding may be sped up by altering one or more hardware parameters, one of more software approaches, or both. In some instances, the decoding scheme may be scaled horizontally or vertically. The one or more hardware parameters may comprise, by way of non-limiting example, clock speed, cores, cache size, RAM size, CPUs, a component in FIG. 10, or any other hardware parameter known in the art, or combination of parameters. The one or more software approaches may comprise implementation using, by way of non-limiting example, concurrency, parallelism, a distributed approach, or any other approach known in the art.

In some instances, the inner codec comprises a decoding scheme comprising a greedy algorithm. In some instances, the inner codec comprises a decoding scheme comprising a maximum likelihood (ML) algorithm. In some instances, the inner codec comprises a decoding scheme comprising a mixed greedy ML algorithm.

A decoding scheme comprising a greedy algorithm (e.g., greedy decoder) is exemplary illustrated in FIG. 8. As shown, a greedy algorithm takes into account transitions from only the most probably state as it decodes each bit position in a sequence. In some instances, each bit is guessed using the greedy algorithm one at a time. In some instances, more than one bit is guessed using the greedy algorithm at a given time. In some instances, the x-axis comprises the bit position and the y-axis comprises a state. In some instances, a state comprises one or more valid encoding states S that are analyzed at each bit position. In some instances, each state S is assigned a probability. In some instances, the state S is defined as the encoded bits from each lane, a bit history, and a bit position. In some instances, the state S is defined as the bit history and the bit word. The greedy algorithm repeatedly finds the highest probable state at each position until the highest probable end state is reached. In some instances, the decoded bits are backtracked by following the highest probable states at each bit position. In some instances, this results in the fully decoded bit. In some cases, the greedy decoder finds a locally optimal solution. In some instances, the locally optional solution is an approximate of a globally optimal solution. The greedy decoder provides a solution (or end state) in a reasonable amount of time compared to other decoding schemes, such as those described herein.

In some examples, the greedy decoder can correct for errors up to 6% deletions, 4% mutations, or 1% insertions, or any combination thereof. In some examples, the greedy decoder can correct for errors up to 3% deletions, 2% mutations, or 0.5% insertions, or any combination thereof. In some examples, the greedy decoder can correct for errors up to 3% deletions, 2% mutations, or 0.5% insertions, or any combination thereof. In some examples, the greedy decoder can correct for errors of about 1%, 2%, 3%, 4%, 5%, or 6% deletions; about 1%, 2%, 3%, or 4% mutations; or about 0.5% or 1% insertions; or any combination thereof. In some examples, the greedy decoder can correct for errors of about 1%, 2%, 3%, 4%, 5%, or 6% deletions; about 1%, 2%, 3%, or 4% mutations; or about 0.5% or 1% insertions; or any combination thereof.

In some instances, performance of the decoding scheme is improved by knowing where the polynucleotide sequence ends. In some cases, the oligonucleotide lengths are determined during sequencing, for example, through pair-end sequencing. In some instances, a drift term is introduced to the greedy algorithm. The drift term comprises an integer associated with the total number of insertions and deletions. Each insertion is represented as a +1 value and each deletion is represented as a-1 value. For example, if there are no insertions and 2 deletions, the total drift is-2. In such an example, the greedy algorithm discards all end decoding states that do not match the length of oligo as being invalid. Therefore, the drift term allows the greedy algorithm to know which end decoding states are valid, and can further improve the performance. As such, in some instances, as shown in FIG. 8 and FIG. 9, the decoding scheme further comprises a z-axis corresponding to the drift.

A decoding scheme (or an inner codec) comprising a ML algorithm (or ML decoder) is exemplary illustrated in FIG. 9. As shown, a ML algorithm takes into account transitions from all states as it decodes each bit position in a sequence. The states are defined as previously described herein. In some instances, each bit is guessed using the ML algorithm one at a time. In some instances, more than one bit is guessed using the ML algorithm at a given time. In some cases, the ML algorithm repeatedly finds all transition states at each position until end candidate states are determined. In some instances, the x-axis comprises the bit position and the y-axis comprises a state, as previously described herein. In some instances, a drift term, as previously described herein, is used to filter the end candidate states. In some instances, the ML algorithm provides the globally optimal solution by tracking all state transitions. In some cases, the ML algorithm is computationally intensive compared to other decoding schemes, such as those described herein.

In some examples, the ML decoder can correct for errors up to 12% deletions, 6% mutations, or 2% insertions, or any combination thereof. In some examples, the ML decoder can correct for errors up to 6% deletions, 3% mutations, or 1% insertions, or any combination thereof. In some examples, the ML decoder can correct for errors of about 1%, 2%, 3%, 4%, 5%, 6%, 7%, 8%, 9%, 10%, 11%, or 12% deletions; about 1%, 2%, 3%, 4%, 5%, or 6% mutations; or about 0.5%, 1%, 1.5%, or 2% insertions; or any combination thereof. In some examples, the ML decoder can correct for errors of at most about 1%, 2%, 3%, 4%, 5%, 6%, 7%, 8%, 9%, 10%, 11%, or 12% deletions; about 1%, 2%, 3%, 4%, 5%, or 6% mutations; or about 0.5%, 1%, 1.5%, or 2% insertions; or any combination thereof.

In some instances, a decoding scheme of the inner codec comprises a mixed greedy ML algorithm. In some instances, the mixed greedy ML algorithm comprises a greedy algorithm and a ML algorithm. A mixed greedy ML algorithm takes into account transitions from a plurality of states as it decodes each bit position in a sequence. In some instances, the plurality of states are about 100 to about 1000 states as it decodes each bit position in a sequence. In some instances, the plurality of states are about 100 to about 200, about 100 to about 300, about 100 to about 400, about 100 to about 500, about 100 to about 600, about 100 to about 700, about 100 to about 800, about 100 to about 900, about 100 to about 1,000, about 200 to about 300, about 200 to about 400, about 200 to about 500, about 200 to about 600, about 200 to about 700, about 200 to about 800, about 200 to about 900, about 200 to about 1,000, about 300 to about 400, about 300 to about 500, about 300 to about 600, about 300 to about 700, about 300 to about 800, about 300 to about 900, about 300 to about 1,000, about 400 to about 500, about 400 to about 600, about 400 to about 700, about 400 to about 800, about 400 to about 900, about 400 to about 1,000, about 500 to about 600, about 500 to about 700, about 500 to about 800, about 500 to about 900, about 500 to about 1,000, about 600 to about 700, about 600 to about 800, about 600 to about 900, about 600 to about 1,000, about 700 to about 800, about 700 to about 900, about 700 to about 1,000, about 800 to about 900, about 800 to about 1,000, or about 900 to about 1,000 states. In some instances, the plurality of states are about 100, about 200, about 300, about 400, about 500, about 600, about 700, about 800, about 900, or about 1,000 states. In some instances, the plurality of states are at least about 100, about 200, about 300, about 400, about 500, about 600, about 700, about 800, or about 900 states. In some instances, the plurality of states are at most about 200, about 300, about 400, about 500, about 600, about 700, about 800, about 900, or about 1,000 states. The states are defined as previously described herein. In some instances, each bit is guessed using the mixed greedy ML algorithm one at a time. In some instances, more than one bit is guessed using the mixed greedy ML algorithm at a given time. In some instances, the mixed greedy ML algorithm repeatedly finds about 100 to about 1000 transition states at each position until end candidate states are determined. In some instances, a drift term, as previously described herein, is used to filter the end candidate states. In some instances, the mixed greedy ML algorithm provides the globally optimal solution, while being less computationally expensive relative to other decoding schemes, such as the ML algorithm described herein.

In some examples, the mixed greedy ML decoder can correct for errors up to 15% deletions, 10% mutations, or 5% insertions, or any combination thereof. In some examples, the mixed greedy ML decoder can correct for errors up to 12% deletions, 6% mutations, or 2% insertions, or any combination thereof. In some examples, the mixed greedy ML decoder can correct for errors up to 6% deletions, 3% mutations, or 1% insertions, or any combination thereof. In some examples, the mixed greedy ML decoder can correct for errors of about 1%, 2%, 3%, 4%, 5%, 6%, 7%, 8%, 9%, 10%, 11%, 12%, 13%, 14%, or 15% deletions; about 1%, 2%, 3%, 4%, 5%, 6%, 7%, 8%, 9%, or 10% mutations; or about 1%, 2%, 3%, or 4% insertions; or any combination thereof. In some examples, the mixed greedy ML decoder can correct for errors of at most about 1%, 2%, 3%, 4%, 5%, 6%, 7%, 8%, 9%, 10%, 11%, 12%, 13%, 14%, or 15% deletions; about 1%, 2%, 3%, 4%, 5%, 6%, 7%, 8%, 9%, or 10% mutations; or about 1%, 2%, 3%, or 4% insertions; or any combination thereof.

In some instances, the decoding scheme in the inner codec comprises a beam search decoder or a random sampling decoder (e.g., pure sampling decoder, a top-K sampling decoder, etc.). In some cases, a beam search decoder or a random sampling decoder provides a diversity of candidate states compared to a greedy decoder.

In some instances, the inner codec further comprises a checksum. In some instances, the checksum is used to verify data integrity, detect errors, or a combination thereof. In some instances, a checksum is generated using a checksum function or checksum algorithm (e.g., parity byte or parity work (longitudinal parity check), sum complement, position dependent, fuzzy checksum, etc.). Examples of checksum functions or algorithms, include, but are not limited to, BSD checksum (Unix), SYSV checksum (Unix), sum4, sum8, sum16, sum32, fletcher-4, fletcher-8, fletcher-16, fletcher-32, Adler-32, x or 8, Luhn algorithm, Verhoeff algorithm, or Damm algorithm. In some instances, instead of only taking the highest probable path, a few of the best probable paths are considered and tested against the checksum. In some instances, the checksum comprises a RS code (e.g., a small RS code). In such instances, the decoder gives a list of possibilities (e.g., “list decoding”) assuming the user can decide which one it actually is.

In some instances, methods and systems for decoding comprises arranging lanes into frames. In some instances, the decoded lanes from the inner codec are arranged into frames based on the lane index and the frame index 215. In some instances, one or more lanes are missing from a frame, as shown in FIG. 7. In some cases, the lanes are missing due to errors occurred during synthesis or sequencing of the nucleotides. In some cases, about 1% to about 10% of the lanes are missing from a frame. In some cases, about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10% of the lanes are missing from a frame. In some cases, about 1%, about 2%, about 4%, about 6%, about 8%, or about 10% of the lanes are missing from a frame. In some cases, at least about 1%, about 2%, about 4%, about 6%, or about 8% of the lanes are missing from a frame. In some cases, at most about 2%, about 4%, about 6%, about 8%, or about 10% of the lanes are missing from a frame.

In some instances, the inner codec comprises a “format”. In some cases, there is no a-priori information about the size of the data (e.g., binary data) during decoding. Therefore, in some instances, frame index 0 comprises the size of the data. In some instances, after arranging the lanes into frames and/or order the frames, frame 0 is decoded first. The data is then extracted from frame 0 to reject frames outside of the expected data size (e.g., from incorrectly decoded oligos).

In some instances, the inner codec comprises a hash (e.g., SHA-256). In some instances, the hash verifies that the data was correctly decoded. In some instances, by using a hash at the end (after the outer codec or ECC), the encoding and decoding are performed as a stream. In some instances, this can limit memory use to only temporary buffers.

Methods for decoding a plurality of polynucleotide sequences comprise an outer codec or error correction code (ECC). In some instances, the plurality of polynucleotide sequences are decoded into data (e.g., binary data). In some instances, an outer codec or ECC is applied to each of the frames 220. In some instances, the outer codec or ECC is applied to the lanes from the inner codec. In some instances, the outer codec or ECC is applied after the lanes from the inner codec are arranged into frames.

In some instances, the outer codec comprises an error correction scheme or code is based on the error correction scheme used to encode the date (e.g., binary data). In some instances, the error correction scheme comprises a Reed-Solomon (RS) code, a LDPC code, a polar code, a turbo code, or any combination thereof.

In some instances, the error correction scheme of the outer codec comprises a Reed-Solomon (RS) code. In such instances, the RS decoder receives a codeword, r (x), which is the original codeword c (x) plus errors e (x) (e.g., r (x)=c (x)+e (x)). In some cases, the errors e (x) is 0. In some instances, the RS decoder attempts to identify the position and magnitude of up to t errors (or 2t erasures). The RS code then attempts to correct these identified errors and/or erasures.

In some instances, the RS decoder comprises a syndrome calculation. In some instances, the syndrome calculation comprises receiving incoming symbols and dividing them into the generator polynomial g (x), as previously described herein. In some instances, the syndromes are calculated by substituting the 2t roots (or syndromes of the RS codeword c (x)) of the generator polynomial g (x) into r (x). In some instances, the generator polynomial g (x) is a known parameters of the decoder. In some instances, the RS codeword c (x) has 2t syndromes that depend on errors.

In some instances, the RS decoder comprises finding a symbol error location. In some instances, parity or check symbols t cause the syndrome calculation to be zero in the case of no errors. In some instances, parity or check symbols t comprise the remainder in the RS encoder. If there are errors, the resulting polynomial g (x) is passed to a Euclid algorithm. In some instances, factors of the remainder are found using the Euclid algorithm. In some instances, the results are evaluated over iterations for each of the incoming symbols. In some instances, errors are found and the errors are corrected. In some cases, the corrected code word c (x) is the outputted from the RS decoder. In some instances, there are more errors in the code word than can be corrected by the RS code (e.g., e (x)>2t). In such instances, the received codeword r (x) is outputted from the RS decoder. In some instances, the received codeword r (x) is outputted with an indication that the error correction has failed (e.g., a flag). In some instances, the received codeword r (x) (e.g., the lane or the frame comprising binary data as described herein) is discarded.

In some instances, the frames from the outer codec (or ECC) are merged to generate an output comprising the data 225. In some instances, the data comprises binary data, which may be byte streams or byte arrays, as previously described herein.

The decoding methods (e.g., inner codec, outer codec, or both) described herein can be used to recover data in the presence of an error in at least one polynucleotide sequence in the plurality of polynucleotide sequences that was stored. In some instances, the error comprises an insertion, deletion, substitution, or any combination thereof. In some instances, the data is recovered in the presence of errors (e.g., error rate) in about 0.001% to about 30% of the polynucleotide sequences in the plurality of nucleotides. In some instances, the data is recovered in the presence an error rate of about 0.001% to about 0.01%, about 0.001% to about 0.1%, about 0.001% to about 0.5%, about 0.001% to about 1%, about 0.001% to about 2%, about 0.001% to about 5%, about 0.001% to about 10%, about 0.001% to about 15%, about 0.001% to about 20%, about 0.001% to about 25%, about 0.001% to about 30%, about 0.01% to about 0.1%, about 0.01% to about 0.5%, about 0.01% to about 1%, about 0.01% to about 2%, about 0.01% to about 5%, about 0.01% to about 10%, about 0.01% to about 15%, about 0.01% to about 20%, about 0.01% to about 25%, about 0.01% to about 30%, about 0.1% to about 0.5%, about 0.1% to about 1%, about 0.1% to about 2%, about 0.1% to about 5%, about 0.1% to about 10%, about 0.1% to about 15%, about 0.1% to about 20%, about 0.1% to about 25%, about 0.1% to about 30%, about 0.5% to about 1%, about 0.5% to about 2%, about 0.5% to about 5%, about 0.5% to about 10%, about 0.5% to about 15%, about 0.5% to about 20%, about 0.5% to about 25%, about 0.5% to about 30%, about 1% to about 2%, about 1% to about 5%, about 1% to about 10%, about 1% to about 15%, about 1% to about 20%, about 1% to about 25%, about 1% to about 30%, about 2% to about 5%, about 2% to about 10%, about 2% to about 15%, about 2% to about 20%, about 2% to about 25%, about 2% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%. In some instances, the data is recovered in the presence an error rate of about 0.001%, about 0.01%, about 0.1%, about 0.5%, about 1%, about 2%, about 5%, about 10%, about 15%, about 20%, about 25%, or about 30%. In some instances, the data is recovered in the presence an error rate of at least about 0.001%, about 0.01%, about 0.1%, about 0.5%, about 1%, about 2%, about 5%, about 10%, about 15%, about 20%, or about 25%. In some instances, the data is recovered in the presence an error rate of at most about 0.01%, about 0.1%, about 0.5%, about 1%, about 2%, about 5%, about 10%, about 15%, about 20%, about 25%, or about 30%.

In some instances, the decoding scheme (e.g., the outer and inner decoding) is used with soft decoding. Soft decoding generally refers to decoding by considering a range of possible values (e.g., using probability estimates). As an example, sequencing can carry the quality for each base, which can be considered during probability calculations. In such an example, each state comprises a final probability, which can be used in the outer decoder as, for example, a log-likelihood, if that outer decoder supports soft-decoding. Further, clustering and alignment can provide soft information on the alignment confidence. As a further example, an LDPC outer codec comprises an iterative decoder. This provides possibilities to go back and forth between the inner and outer decoder in an iterative manner instead of a single pass. However, in some instances, this is accompanied by the cost of higher computing requirements. Decoding can be run on at least one logic element, programmable logic, or processors. Non-limiting examples of at least one logic element, programmable logic, or processors include a programmable logic controller (PLC), programable logic array (PLA), programmable array logic (PAL), generic logic array (GLA), complex programmable logic decide (CPLD), field programable gate array (FPGA), or application-specific integrated circuit (ASIC), GPU, CPU, AI-accelerator or any combination thereof. In some instances, an AI-accelerator comprise Google-TPU, Graphcore, Cerebras, SambaNova, or a combination thereof. In some instances, decoding is run on compute-on-memory technologies, such as, but not limited to, UpMem.

The hashes of the present disclosure can allow verification of digital information during retrieval. In some cases, retrieving a digital information stored in a plurality of polynucleotides further comprises verifying at least the one or more objects 1315. In some instances, the one or more objects are verified using a first one or more hashes in the plurality of pools. In some cases, retrieving a digital information stored in a plurality of polynucleotides further comprises verifying one or more pool items. In some instances, the one or more pool items are verified using a second one or more hashes in the plurality of pools. In some examples, if an object is stored across more than one pool of the plurality of pools, more than one pool item is assembled into this object. In such examples, the first one or more hashes of the data payload of each of the pool items, the second one or more hashes of one or more objects, or a combination thereof enables proper assembly verification.

Verifying hashes generally comprises generating hashes (e.g., cryptographic hashes). Verifying can further comprise comparing the generated hashes with the previously determined hashes. In some cases, the previously hashes and the new hashes are determined using the same hash function. In some instances, the hash function comprises a cryptographic hash function. In some cases, the hash function comprises MD-5, SHA-1, SHA-2, SHA-3, RIPEMD-160, Whirlpool, BLAKE, BLAKE2, BLAKE3, or a variation thereof. In some instances, the hash function comprises SHA-2. In some examples, SHA-2 comprises SHA-224, SHA-256, SHA-384, SHA-512, SHA-512/224, or SHA-512/256. In some cases, if the new and previous hashes match, the integrity of the item of information (e.g., an object) is verified. In some cases, if the new and previous hashes do not match, verification fails. In some instances, if verification fails, the integrity of the item of information is not verified. In some instances, if verification fails, the item of information has been modified and/or corrupted.

Retrieving digital information can comprise combining the information stored across pools items and/or the plurality of pools. In some cases, retrieving a digital information stored in a plurality of polynucleotides further comprises combining the digital information in the plurality of pools 1320. In some instances, the data payload in the one or more pool items are combined. In some instances, the data payload in the one or more pool items across the plurality of pools are combined. In some instances, the combined data payloads comprise the digital information. In some cases, the retrieved data or digital information is stored on a memory 1325.

In some cases, the retrieved digital information is presented to a user. In some instances, the information is presented to a user on an interface. In some instances, the interface is an interface of an electronic device (e.g., personal electronic device). In some instances, the electronic device comprises an application configured to communicate with the systems described herein via a computer network to access the information.

In some instances, the methods to decode a plurality of polynucleotide sequences to generate an output comprising digital data (e.g., binary data), as described herein, are performed on a system. In some instances, the system performs the operations generally illustrated in FIG. 1, FIG. 2, or both. In some instances, such a system comprises an apparatus comprising a memory, a sequencing device, a processing device operatively coupled to the memory, or a combination thereof. In some instances the sequencing device is operatively coupled to the memory, the processing device, or the combination thereof. In some instances, the memory is used to store information of the binary data, the polynucleotide sequences, or the combination thereof. In some instances, the information of the binary data, the polynucleotide sequences, or the combination thereof is from one or more step in the encoding methods described herein. In some instances, the memory is used to store information related to the algorithms described herein (e.g., software code, parameters, executable instructions, etc.). In some examples, the memory can comprise any suitable memory described herein. In some examples, the memory can be configured according to embodiments described herein. In some examples, the sequencing device is configured to determine the plurality of polynucleotide sequences using the methods described herein.

In some instances, the processing device is configured to perform one or more decoding steps. In some instances, the processing device is configured to perform one or more steps comprising: apply an inner codec comprising a decoding scheme to the plurality of polynucleotide sequences; arrange the lanes of binary data into frames based on a lane index and a frame index in each of the lanes of binary data; and apply an outer codec to the frames. In some instances, the decoding scheme transforms each of the plurality of polynucleotide sequences into lanes of binary data. In some instances, the decoding scheme comprises a mixed decoding algorithm comprising a greedy algorithm and a maximum likelihood (ML) algorithm. In some instances, the outer codec comprises an error correction scheme. In some instances, the frames from the outer codec are merged to generate an output comprising the binary data.

The methods for retrieving digital information in DNA (or polynucleotides) can be carried out on a system. In some instances, the system performs the operations generally illustrated in FIG. 12, FIG. 13, or both. In some cases, such a system comprises an apparatus comprising one or more processing units, a memory, instructions, a sequencing device, or a combination thereof. In some instances, the memory is in communication with the one or more processing units. In some instances, the instructions are stored on the memory. In some instances the sequencing device in communication with the memory, the one or more processing units, or the combination thereof. In some cases, the one or more processing units and memory are distributed across one or more physical or logical locations.

In some instances, the memory is used to store the data or digital information, the polynucleotides sequences (e.g., partially or fully decoded sequences), or the combination thereof. In some instances, the memory is used to store information related to the algorithms described herein (e.g., software code, parameters, executable instructions, etc.). In some examples, the memory can comprise any suitable memory described herein. In some examples, the memory can be configured according to embodiments described herein. In some examples, the sequencing device is configured to determining the plurality of polynucleotide sequences using the methods described herein.

In some cases, the one or more processing units include any combination of central processing units (CPUs), graphical processing units (GPUs), single core processors, multi-core processors, processor clusters, application-specific integrated circuits (ASICs), programmable circuits such as Field Programmable Gate Arrays (FPGA), an AI-accelerator and variations thereof. In some cases, the one or more of the processing units comprise a Single Instruction Multiple Data (SIMD) or Single Program Multiple Data (SPMD) parallel architectures. As an example, the one or more processing units include one or more GPUs or CPUs that implement SIMD or SPMD. In some instances, an AI-accelerator comprise Google-TPU, Graphcore, Cerebras, SambaNova, or a combination thereof. In some embodiments, one or more of the processing units is implemented in software and/or firmware, in addition to hardware implementations. Software or firmware implementations of the processing units can include computer- or machine-executable instructions written in any suitable programming language to perform the various functions described herein. Software implementations of the one or more processing units can be stored in whole or part in the memory. Alternatively or additionally, the system can comprise one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. In some instances, decoding is run on compute-on-memory technologies, such as, but not limited to, UpMem.

In some instances, the one or more processing units is configured to perform one or more decoding steps. In some instances, the processing device is configured to perform one or more steps comprising: applying a decoding scheme to decode the digital information in the plurality of pools; verifying at least the data payload in a pool item using a first one or more hashes in the plurality of pools; combining the digital information in the plurality of pools to retrieve the one or more objects; and storing the digital information on a memory. In some instances, the one or more processing units is configured to perform one or more steps comprising: apply an inner codec to the plurality of polynucleotides; or apply an ECC to the plurality of polynucleotides. In some instances, the inner codec transforms each of the plurality of polynucleotides into digital information. In some instances, the inner codec comprises a mixed decoding algorithm comprising a greedy algorithm and a maximum likelihood (ML) algorithm. In some instances, the output from an ECC are merged to generate an output comprising the digital information.

### DNA Data Storage

Polynucleotides encoding information described herein may be stored in a data storage system. A system for data storage can comprise one or more modules. In some instances, the some or all of the one or more modules are in communication. In some examples, some or all of the one or more modules are in communication to allow transferring of polynucleotides between them. In some examples, some or all of the one or more modules are fluidically coupled. In some examples, some or all of the one or more modules are fluidically coupled with one or more tubes. A fluid may generally refer to one or more liquids used in various processes involved in handling polynucleotides, including, without limitation, synthesis, amplification, preparation for sequencing, and sequencing. In some examples, some or all of the modules are in communication to allow transferring of control commands between modules of the system. In some examples, some or all of the one or more modules are electronically coupled. A module in the system can comprise, without limitation, a synthesizer unit, an amplification chamber, a sequencer unit, a storage unit, a controller, a robotic system, or any combination thereof. In some examples, a module can further comprise a fluid source, a database or a file system, or both. In some examples, the database or file system keeps track of the storage capacity of the system. For example, the database or file system can keep track of available racks (or trays), slots (for capsules), or both. In some examples, the database or the file system is used to determine the disposition of the rack within the storage system. In some instances, movement of polynucleotides between one or more modules of a system is accomplished by one or more tubes or a robotic system. In some examples, the database or the file system is used to direct the robotic system to the correct position in the storage system. In some instances, the system is autonomous.

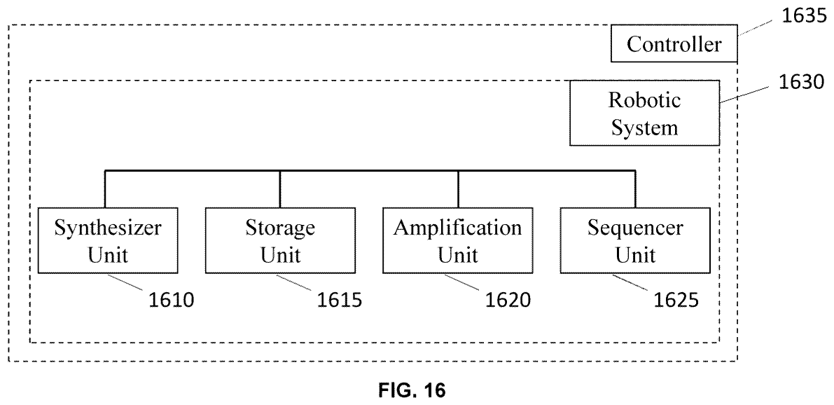

A non-limiting example of a system for data storage is illustrated in FIG. 16. A system for data storage may comprise a synthesizer unit 1610. A synthesizer unit can be used to synthesize a plurality of polynucleotides encoding digital information. In some instances, the system comprises more than one synthesizer units 1610. Polynucleotides may be synthesized using a method provided herein or any other suitable synthesis method known in the art. The fluidic and/or electronic control of polynucleotide synthesis in the synthesizer unit 1610 may be performed by a controller 1635. In some instances, the electronics in the synthesizer unit 1610 are in communication with the controller 1635. In some instances, the synthesizer unit 1610 has an input for receiving DNA sequences. In some instances, the synthesizer unit 1610 has an input for receiving fluids for polynucleotide synthesis. In some instances, the synthesizer unit 1610 has an output for eluting synthesized polynucleotides. In some instances, the synthesized polynucleotides are transferred to another component of the system, such as, by way of non-limiting example, a storage unit, an amplification chamber, or a sequencing unit.

A synthesizer unit may comprise a solid support. The solid support may comprise a device for polynucleotide storage described herein. The solid support may comprise a surface for polynucleotide synthesis. In some instances, the solid support, the surface, or both comprise a material described herein. In some instances, the material comprises a metal or organic polymer. In some instances, the material comprises steel (e.g., stainless steel) or other metal alloy. In some instances, the material comprises polyethylene, polypropylene, or other polymer. In some instances, the struture comprises a flexible material, such as those provided herein. Exemplary flexible materials include, without limitation, modified nylon, unmodified nylon, nitrocellulose, and polypropylene. In some instances, the materials comprise a rigid material, such as those provided herein. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, steel, gold, platinum). In some instances, materials disclosed herein may be fabricated from a material comprising silicon, polystyrene, agarose, dextran, cellulosic polymers, polyacrylamides, polydimethylsiloxane (PDMS), glass, or any combination thereof. In some examples, materials disclosed herein are manufactured with a combination of materials listed herein or any other suitable material known in the art.

In some instances, the polynucleotides are deprotected, cleaved, and/or eluted from the synthesizer unit 1610 and transferred to another module in the system. In some instances, a robotic system 1630 or fluidic tube is used to transports the polynucleotides to another module in the system. A robotic system 1630 may be controlled by a controller 1635. A robotic system generally comprises a system for manipulation of a plurality of polynucleotides. In some instances, the robotic system is used to manipulate a structure comprising a plurality of polynucleotides, such as those described herein. Manipulation can comprise, by way of non-limiting example, moving, storing, retrieving, handling, transferring, or any combination thereof. The robotic system may be similar to those used in semiconductor processing to move trays of wafers and chips between processing devices. A robotic system 1630 may be used to select and transfer polynucleotides between modules of the system. For example, a robotic system 1635 may include a tag reader to verify a structure in a storage unit 1615. In some instances, the robotic system 1635 comprises a tag reader and the structure in the storage unit 1615 comprises a tag (e.g., barcode or RFID tag). Once verified, the robotic system 1630 may transfer the structure to a component of the system. Additionally, the robotic system 1630 may transfer the structure to a precise location in a component of the system. In some instances, the robotic system can allow for polynucleotides to be added and/or removed from modules in the data storage system. In some instances, the robotic system allows for a structure comprising a plurality of polynucleotides to be placed and/or retrieved from a location in an identifiable layout in the storage unit 1615. The robotic system 1630 may be controlled using a controller 1635 as further described herein.

In some instances, one or more droplets comprising polynucleotides are transferred from a synthesizer unit 1610 to a storage unit 1615. In some instances, some or all of the polynucleotides synthesized on a solid support are transferred to a structure for storage. The structure or compartments may have a variety of shapes and sizes. The structure may further comprise a tag (e.g., barcode or an RFID tag). In some instances, a plurality of polynucleotides are transferred to a structure in the synthesizer unit 1610. In some instances, a plurality of polynucleotides are transferred to a structure in the storage unit 1615. The fluidic and/or electronic control of polynucleotide synthesis in the storage unit 1615 may be performed by a controller 1635. In some instances, the electronics in the storage unit 1615 are in communication with the controller 1635. In some instances, the polynucleotides are stored at room temperature in the storage unit 1615. In some instances, the system comprises a database or a file system for keeping track of the storage capacity in the storage unit 1615. In some examples, the database comprises a control application database. In some instances, the database or the file system is part of the controller 1635.

A structure comprising a plurality of polynucleotides can be stored in an identifiable layout in storage unit 1615. The identifiable layout may comprise a rack or a plurality of racks, or a variation thereof. The rack may be used to hold one or more structures comprising the plurality of polynucleotides. In some instances, each structure is stored at a fixed location in the identifiable layout. In some instances, the tag comprises information about a location of the structure in the identifiable layout. As an example, a tag can encode metadata comprising a location of the structure in the identifiable layout. In some instances, the rack may be located in a data center. In some instances, the rack uses mechanical structures commonly used for mounting conventional computing and data storage resources in rack units. For example, a rack may comprise openings adapted to support disk drives, processing blades, and/or other computer equipment. In some instances, a rack comprises a tag. In some examples, the tag comprises information of the structures stored in/on the rack. In some examples, the tag comprises a list of the structures stored in/on the rack.

In some instances, the storage unit 1615 may be accessed using a robotic system 1630. In some instances, the identifiable layout in the storage unit 1615 comprises robotically addressable slots. Each slot may hold a structure comprising a plurality of polynucleotides. In some instances, each slot comprises a width, depth, length, or any combination thereof for accommodating a structure comprising the plurality of polynucleotides. In some instances, a rack comprises a plurality of slots, where each slot holds a structure comprising the plurality of polynucleotides.

The system for storing polynucleotides may further comprise an amplification chamber 1620. The amplification unit may be used to amplify the plurality of polynucleotides. In some instances, the system comprises more than one amplification chamber 1620. In some instances, a structure is selected from a storage unit 1615 and the polynucleotides from the structure are transferred to the amplification chamber 1620. In some instances, the polynucleotides from a synthesizer unit 1610 are transferred to the amplification chamber 1620 for size selection, PCR, or other type of amplification or preparation for storage. Size selection generally involves selecting DNA in the target size and rejecting strands that are much shorter or much longer. In some instances, filters are tuned to capture DNA of a particular size range. In some instances, other methods include PCR, electrophoresis, capture by solid phase bound primers, which are complementary to the end sequences of synthesized oligonucleotides, or the use of an isothermal polymerase. The fluidic and/or electronic control of polynucleotide synthesis in the amplification chamber 1620 may be performed by a controller 1635. In some instances, the electronics in the amplification chamber 1620 are in communication with the controller 1635.

The system for storing polynucleotides may further comprise a sequencing unit 1625. The sequencing unit 1625 may be used to sequence a plurality of polynucleotides. In some instances, the plurality of polynucleotides are transferred from the amplification chamber 1620 to the sequencing unit 1625. In some instances, the system may comprise additional modules for performing additional sequencing preparation steps. In some examples, the plurality of polynucleotides are transferred from the amplification chamber 1620 to the sequencing unit 1625 using one or more tubes or the robotic system 1630. In some instances, the amplification chamber 1620 and the sequencing unit 1625 are fluidically coupled. The fluidic and/or electronic control of polynucleotide synthesis in the sequencing unit 1625 may be performed by a controller 1635. In some instances, the electronics in the sequencing unit 1625 are in communication with the controller 1635.

In some instances, the system comprises large-scale sequencing of polynucleotides. In some instances, large-scale sequencing comprises dense and highly parallel sequencers. In some instances, the system comprises more than one sequencing unit 1625. In some instances, the sequencing unit 1625 use centrifugal forces and/or vacuum/pressure to add or evacuate reagents from the sequencing unit 1625. In some instances, the sequencing unit 1625 is light-based (e.g., with light sources and sensors on chip), nanopore-based (e.g., Oxford Nanopore Technologies (ONT)), or involve other operations (e.g., a light-based method such as PacBio or other sequencing technologies). In some instances, the sequencing unit 1625 employs sequencing methods provided herein. In some instances, the sequencing unit 1625 uses of nanopores or other electrical sequencing technology that benefits from the bulk fluidics provided by semiconductor fabrication equipment. In some instances, the one or more modules described herein comprises a camera. A camera may be used to capture one or more optical features of polynucleotides in a module. As an example, a camera may be used in a synthesizer unit, a sequencing unit, or both, to capture an optical feature of polynucleotides attached to a surface on a solid support as described herein.

The system for storing polynucleotides can comprise a robotic system 1630 as described herein. The robotic system may generally be used to manipulate the polynucleotides in a system. Manipulation can comprise, without limitation, moving, storing, retrieving, handling, transferring, or any combination thereof. In some instances, the robotic system transfers the plurality of polynucleotides between modules in the system. In some examples, the robotic system manipulates (e.g., transfers) the plurality of polynucleotides in structure for storage as described herein. In some instances, the robotic system manipulates (e.g., transfers) the plurality of polynucleotides in a rack. In some examples, the rack comprises a plurality of structures each comprising a tag. In some examples, the rack comprises a plurality of solid supports for synthesis and/or sequencing. In some instances, the robotic system comprises a robotic hand or a robotic picker. In some instances, the robotic system 1630 is fully integrated with the storage system control software and/or firmware in the controller 1635. In some instances, the robotic system 1630 is fully integrated with an external host application. In some instances, the robotic system 1630 is fully automated.

The system for storing polynucleotides can comprise a controller 1635. The controller may generally be used for controlling modules, components, fluidics, robots, or any combination thereof. The modules, components, fluidics, electronics, robots, or any combination thereof may be used for synthesizing, storing, retrieving, sequencing, and/or amplifying polynucleotides. In some instances, the controller 1635 is capable of cataloguing all storage structures loaded, unloaded, and/or stored within a rack. The polynucleotides can encode digital information as described herein. The modules, components, fluidics, electronics, robots, or any combination thereof may be used for performing methods, models, or algorithms, such as encoding or decoding the polynucleotides.

In some instances, the controller 1635 controls the physical location of the plurality of polynucleotides. In some instances, the controller 1635 provides commands to one or more modules of the system. In some examples, the controller 1635 controls robotics (e.g., robotic system 1630), actuators, and fluidic valves, or any other equipment of the system. In some instances, the controller 1635 allows for synchronizing and controlling the modules for processing and/or transferring polynucleotides. In some examples, the polynucleotides are processed and/or transferred via fluidics. In some examples, the polynucleotides are processed and/or transferred via electronics. In some instances, the controller 1635 controls physical parameters in one or more modules, such as, without limitation, pressure, vacuum, temperature, volume (e.g., of fluids), or any combination thereof.

In some instances, the controller 1635 invokes an encoder module or a decoder module. In some instances, the encoder module encodes the digital information as a plurality of polynucleotides. In some instances, the encoder module applies one or more codecs, such as those described herein (e.g., FIG. 1, FIGS. 3-6, FIG. 12, FIGS. 13-14), to the digital information. In some instances, the decoder module decodes the sequences of the plurality of polynucleotides to retrieve the digital information. In some instances, the decoder module applies one or more codecs, such as those described herein (e.g., FIG. 2, FIGS. 7-9, FIG. 13), to the sequences of the plurality of polynucleotides. In some instances, the decode module performs reassembly, error correction, and outputs digital information (e.g., binary data). In some instances, the output comprising digital information is transferred to an operating system and/or a file system. The output may be provided on a display, such as a graphical user interface (GUI), or any other suitable display such as those described herein, for providing the digital information. In some instances, the controller 1635 is implemented on one or more software modules, such as those described herein. In some instances, the controller 1635 responds to commands from an operating system, such as those described herein.

### Certain Definitions

Unless otherwise defined, all technical terms used herein have the same meaning as commonly understood by one of ordinary skill in the art to which the present subject matter belongs.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Reference throughout this specification to “some instances,” “further instances,” or “a particular instance,” means that a particular feature, structure, or characteristic described in connection with the instance is included in at least one instance. Thus, the appearances of the phrase “in some instances,” or “in further instances,” or “in a particular instance” in various places throughout this specification are not necessarily all referring to the same instance. Furthermore, the particular features, structures, or characteristics may be combined in any suitable manner in one or more instances.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the polynucleotide being known and chosen before the synthesis or assembly of the nucleic acid molecules.

As used herein, the term “hash” or “hashes” may generally refer to a string of fixed length that is outputted from a hash function. A hash function may generally comprise a function that receives an input of arbitrary length into an output with a fixed length. In some instances, the input may be one or more bits, which may be passed through hash function to generate a hash. In some instances, the hash function may be deterministic, and it may be infeasible to reverse-engineer the input from the hashed output. The act of feeding an input into a hash function may be referred to as “hashing”.

The term “symbol,” as used herein, generally refers to a representation of a unit of digital information. Digital information may be divided or translated into one or more symbols. In an example, a symbol may be a bit and the bit may have a numerical value. In some examples, a symbol may have a value of ‘0’ or ‘1’. In some examples, digital information may be represented as a sequence of symbols or a string of symbols. In some examples, the sequence of symbols or the string of symbols may comprise binary data.

Polynucleotide sequences described herein may be, unless stated otherwise, comprise DNA or RNA or an analog or derivative thereof. As used herein, the terms nucleic acids, nucleotides polynucleotides, oligonucleotides, oligos, oligonucleic acids are used synonymously throughout to represent a polymer of nucleoside monomers. As used herein, the terms nucleic acid sequences, polynucleotide sequences, polynucleotide sequences, oligonucleotides sequences, oligo sequences or oligonucleic acid sequences are also used synonymously throughout to represent the sequences of a polymer of nucleoside monomers. In some instances, nucleic acids are connected via phosphate or sulfur-containing linkages. Nucleic acids in some instances comprise DNA, RNA, non-canonical nucleic acids, unnatural nucleic acids, or other nucleoside. In some instances, nucleotides comprise non-canonical bases, sugars, or other moiety. In some instances, nucleotides comprise terminators which are configured to prevent extension reactions. In some instances, such terminators are removed before addition of subsequent nucleotides to the growing chain.

### Computing System

Referring to FIG. 10, a block diagram is shown depicting an exemplary machine that includes a computer system 1000 (e.g., a processing or computing system) within which a set of instructions can execute for causing a device to perform or execute any one or more of the aspects and/or methodologies for static code scheduling of the present disclosure. The components in FIG. 10 are examples only and do not limit the scope of use or functionality of any hardware, software, embedded logic component, or a combination of two or more such components implementing particular embodiments.

A platform comprising a computer system as shown in FIG. 10 may be used for encoding data represented as a set of symbols to another set of symbols. In some instances, the computer system converts a first string of symbols to a second string of symbols using the program. In some instances, the computer system executes a program to convert the data to a plurality of polynucleotide sequences, convert a plurality of polynucleotide sequences to data, or both. As an example, a computing system as generally illustrated in FIG. 10 may be used to execute one or more software programs for encoding a string of symbols (representing an item of information) into polynucleotide sequences (e.g., one or more of the methods illustrated in FIG. 1, 3-6, 12, or 14-15), decoding the polynucleotide sequences back to the string of symbols (e.g., one or more of the methods illustrated in FIG. 2, 7-9, or 13), or both. More specifically, the data may be represented as numerical symbols, such as binary values of “0”s and “1”'s and the computer system 1000 may execute a computer program (e.g., inner codec, outer codec, or both) to convert the data to a plurality of polynucleotide sequences. In some instances, the computer system executes a program to convert a first one or more polynucleotide sequence to a second one or more polynucleotide sequences.

A platform for encoding data can further comprise one or more components, such as a synthesizer, a sequencer, a storage unit, or any combination thereof. In such a platform, in some instances, the computer system 1000 is in electronic communication with any one of the one or more components, such as the synthesizer, the sequencer, the storage unit, or any combination thereof. In some instances, the one or more components are operably linked to a computer system and are optionally automated through a computer either locally or remotely. In various instances, the methods and systems described herein further comprise software programs for the operations of one or more components of the platform on computer systems and use thereof. Accordingly, computerized control for the synchronization of the dispense/vacuum/refill functions such as orchestrating and synchronizing the material deposition device movement, dispense action and vacuum actuation are within the bounds of the disclosure provided herein. In some instances, the computer systems are programmed to interface between the user specified base sequence and the position of a material deposition device to deliver the correct building blocks and/or reagents to specified regions of the substrate (e.g., specific loci). Further, a computer system, such as the system shown in FIG. 10, may be used for monitoring one or more components in platform. For example, the computer system may be used to monitor one or more sensor data from a sensor integrated in or connected to a component. In some instances, the computer system employs a program to monitor and detect irregularities in one or more parameters, such as pressure, volume, flow rate, temperature, vacuum, angles of orientation, humidity, or any other physical parameters that can be measured in the systems and platforms described herein. The computer system comprising the program may analyze patterns in one or more sensor data and optionally alert a user through an HMI if any irregularities are detected or if any data or combination of data fall outside of a threshold (e.g., predetermined or dynamic thresholds).

A program may be executed on a computer system provided herein. In some instances, a program comprises a statistical algorithm or a machine learning algorithm. In some instances, an algorithm comprising machine learning (ML) is trained to perform the functions or operations described herein. In some cases, the algorithm comprises classical ML algorithms for classification and/or clustering (e.g., K-means clustering, mean-shift clustering, density-based spatial clustering of applications with noise (DBSCAN), expectation-maximization (EM) clustering, agglomerative hierarchical clustering, logistic regression, naïve Bayes, K-nearest neighbors, random forests or decision trees, gradient boosting, support vector machines (SVMs), or a combination thereof).

In some cases, the algorithm comprises a learning algorithm comprising layers, such as one or more neural networks. Neural networks may comprise connected nodes in a network, which may perform functions, such as transforming or translating input data. In some examples, the output from a given node may be passed on as input to another node. In some embodiments, the nodes in the network may comprise input units, hidden units, output units, or a combination thereof. In some cases, an input node may be connected to one or more hidden units. In some cases, one or more hidden units may be connected to an output unit. The nodes may take in input and may generate an output based on an activation function. In some embodiments, the input or output may be a tensor, a matrix, a vector, an array, or a scalar. In some embodiments, the activation function may be a Rectified Linear Unit (ReLU) activation function, a sigmoid activation function, or a hyperbolic tangent activation function. In some embodiments, the activation function may be a Softmax activation function. The connections between nodes may further comprise weights for adjusting input data to a given node (e.g., to activate input data or deactivate input data). In some embodiments, the weights may be learned by the neural network. In some embodiments, the neural network may be trained using gradient-based optimizations. In some cases, the gradient-based optimization may comprise of one or more loss functions. In some examples, the gradient-based optimization may be conjugate gradient descent, stochastic gradient descent, or a variation thereof (e.g., adaptive moment estimation (Adam)). In further examples, the gradient in the gradient-based optimization may be computed using backpropagation. In some embodiments, the nodes may be organized into graphs to generate a network (e.g., graph neural networks). In some embodiments, the nodes may be organized into one or more layers to generate a network (e.g., feed forward neural networks, convolutional neural networks (CNNs), recurrent neural networks (RNNs), etc.). In some cases, the neural network may be a deep neural network comprising of more than one layer.

In some cases, the neural network may comprise one or more recurrent layer. In some examples, the one or more recurrent layer may be one or more long short-term memory (LSTM) layers or gated recurrent unit (GRU), which may perform sequential data classification and clustering. In some embodiments, the neural network may comprise one or more convolutional layers. The input and output may be a tensor representing of variables or attributes in a data set (e.g., features), which may be referred to as a feature map (or activation map). In some cases, the convolutions may be one dimensional (1D) convolutions, two dimensional (2D) convolutions, three dimensional (3D) convolutions, or any combination thereof. In further cases, the convolutions may be 1D transpose convolutions, 2D transpose convolutions, 3D transpose convolutions, or any combination thereof. In some examples, one-dimensional convolutional layers may be suited for time series data since it may classify time series through parallel convolutions. In some examples, convolutional layers may be used for analyzing a signal (e.g., sensor data) from one or more components of a system described herein.

The layers in a neural network may further comprise one or more pooling layers before or after a convolutional layer. The one or more pooling layers may reduce the dimensionality of the feature map using filters that summarize regions of a matrix. This may down sample the number of outputs, and thus reduce the parameters and computational resources needed for the neural network. In some embodiments, the one or more pooling layers may be max pooling, min pooling, average pooling, global pooling, norm pooling, or a combination thereof. Max pooling may reduce the dimensionality of the data by taking only the maximums values in the region of the matrix, which helps capture the significant feature. In some embodiments, the one or more pooling layers may be one dimensional (1D), two dimensional (2D), three dimensional (3D), or any combination thereof. The neural network may further comprise of one or more flattening layers, which may flatten the input to be passed on to the next layer. In some cases, the input may be flattened by reducing it to a one-dimensional array. The flattened inputs may be used to output a classification of an object (e.g., classification of signals (e.g., sensor data) in a system described herein). The neural networks may further comprise one or more dropout layers. Dropout layers may be used during training of the neural network (e.g., to perform binary or multi-class classifications). The one or more dropout layers may randomly set certain weights as 0, which may set corresponding elements in the feature map as 0, so the neural network may avoid overfitting. The neural network may further comprise one or more dense layers, which comprise a fully connected network. In the dense layer, information may be passed through the fully connected network to generate a predicted classification of an object, and the error may be calculated. In some embodiments, the error may be backpropagated to improve the prediction. The one or more dense layers may comprise a Softmax activation function, which may convert a vector of numbers to a vector of probabilities. These probabilities may be subsequently used in classifications, such as classifications of signal (e.g., sensor data) from a system described herein, or probable nucleobases during decoding (e.g., as part of a codec).

Computer system 1000 may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 1035, and various tangible storage media 1036. All of these elements may interface directly or via one or more interfaces or adaptors to the bus 1040. For instance, the various tangible storage media 1036 can interface with the bus 1040 via storage medium interface 1026. Computer system 1000 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1000 includes one or more processor(s) 1001 (e.g., central processing units (CPUs), general purpose graphics processing units (GPGPUs), or quantum processing units (QPUs)) that carry out functions. Processor(s) 1001 optionally contains a cache memory unit 1002 for temporary local storage of instructions, data, or computer addresses. Processor(s) 1001 are configured to assist in execution of computer readable instructions. Computer system 1000 may provide functionality for the components depicted in FIG. 10 as a result of the processor(s) 1001 executing non-transitory, processor-executable instructions embodied in one or more tangible computer-readable storage media, such as memory 1003, storage 1008, storage devices 1035, and/or storage medium 1036. The computer-readable media may store software that implements particular embodiments, and processor(s) 1001 may execute the software. Memory 1003 may read the software from one or more other computer-readable media (such as mass storage device(s) 1035, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random access memory component (e.g., RAM 1004) (e.g., static RAM (SRAM), dynamic RAM (DRAM), ferroelectric random access memory (FRAM), phase-change random access memory (PRAM), etc.), a read-only memory component (e.g., ROM 1005), and any combinations thereof. ROM 1005 may act to communicate data and instructions unidirectionally to processor(s) 1001, and RAM 1004 may act to communicate data and instructions bidirectionally with processor(s) 1001. ROM 1005 and RAM 1004 may include any suitable tangible computer-readable media described below. In one example, a basic input/output system 1006 (BIOS), including basic routines that help to transfer information between elements within computer system 1000, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1008 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 1035 may be removably interfaced with computer system 1000 (e.g., via an external port connector (not shown)) via a storage device interface 1025. Particularly, storage device(s) 1035 and an associated machine-readable medium may provide non-volatile and/or volatile storage of machine-readable instructions, data structures, program modules, and/or other data for the computer system 1000. In one example, software may reside, completely or partially, within a machine-readable medium on storage device(s) 1035. In another example, software may reside, completely or partially, within processor(s) 1001.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1000 may also include an input device 1033. In one example, a user of computer system 1000 may enter commands and/or other information into computer system 1000 via input device(s) 1033. Examples of an input device(s) 1033 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1033 may be interfaced to bus 1040 via any of a variety of input interfaces 1023 (e.g., input interface 1023) including, but not limited to, serial, parallel, game port, USB, FIREWIRE, THUNDERBOLT, or any combination of the above.

In particular embodiments, when computer system 1000 is connected to network 1030, computer system 1000 may communicate with other devices, specifically mobile devices and enterprise systems, distributed computing systems, cloud storage systems, cloud computing systems, and the like, connected to network 1030. The cloud computing systems can comprise a private cloud, a public cloud, a hybrid cloud, a multicloud, or any combination thereof. The cloud computing systems can comprise an infrastructure as a service (IaaS), platform as a service (PaaS), software as a service (SaaS), or any combination thereof. Communications to and from computer system 1000 may be sent through network interface 1020. For example, network interface 1020 may receive incoming communications (such as requests or responses from other devices) in the form of one or more packets (such as Internet Protocol (IP) packets) from network 1030, and computer system 1000 may store the incoming communications in memory 1003 for processing. Computer system 1000 may similarly store outgoing communications (such as requests or responses to other devices) in the form of one or more packets in memory 1003 and communicated to network 1030 from network interface 1020. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

Information and data can be displayed through a display 1032. Examples of a display 1032 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1032 can interface to the processor(s) 1001, memory 1003, and fixed storage 1008, as well as other devices, such as input device(s) 1033, via the bus 1040. The display 1032 is linked to the bus 1040 via a video interface 1022, and transport of data between the display 1032 and the bus 1040 can be controlled via the graphics control 1021. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1032, computer system 1000 may include one or more other peripheral output devices 134 including, but not limited to, an audio speaker, a printer, a storage device, and any combinations thereof. Such peripheral output devices may be connected to the bus 1040 via an output interface 1024. Examples of an output interface 1024 include, but are not limited to, a serial port, a parallel connection, a USB port, a FIREWIRE port, a THUNDERBOLT port, and any combinations thereof.

In addition or as an alternative, computer system 1000 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. In some instances, the computer program is scaled vertically or horizontally. For example, the computer program may be scaled up or down using one or more hardware parameters (e.g., clock speed, cores, cache size, RAM size, CPUs, a component in FIG. 10, etc.), one or more software approaches (e.g., concurrency, parallelism, a distributed approach, etc.), or both.

In some embodiments, instructions executable by one or more processor(s) comprise an encoding or decoding method described herein. In some instances, the encoding or decoding method comprise one or more operations or the general approaches provided in FIGS. 1-9 or FIGS. 12-15. For example, instructions executable by one or more processor(s) may comprise generating an inner codec comprising a codebook. In some instances, the codebook is generated with a base order. In some instances, the base order in selected by the user, the computer program, or both. In some instances, instructions executable by one or more processor(s) comprise applying an inner codec to encode data represented as a set of symbols to another set of symbols. As an example, the data may be represented as numerical symbols, such as binary values of “0”s and “1”'s and the computer program may apply the inner codec to convert the data to a plurality of polynucleotide sequences.

The computer system comprising the computer program may be in electronic communication with one or more components of a platform, such as for example, a synthesizer, a sequencer, or a storage unit. In such instances, the computer program may further execute instructions that cause the system to perform one or more operations. The operation can comprise having the synthesizer generate the plurality of polynucleotides, having the sequencer sequence the plurality of polynucleotides, or transferring the plurality of polynucleotides between the synthesizer, the sequencer, the storage unit, or any combination thereof. In some instances, the computer system receives a plurality of output sequences. In such instances, instructions executable by one or more processor(s) comprise decoding a plurality of output sequences.

Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing system. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### EXAMPLES

The following illustrative examples are representative of embodiments of the software applications, systems, and methods described herein and are not meant to be limiting in any way.

### Example 1-Encoding Binary Data in Oligonucleotides Using Lane Index Shuffling

A 4.5 GB data stream is divided into 100,000 frames, where each frame has 45 KB per frame. Each frame is divided into 2500 lanes and each lane has 144 bits. An outer Reed-Solomon GF (212) code is applied to each frame, which increases the size of the frame and generates 4095 lanes per frame.

Each lane is then shuffled using a rotation scheme. The rotation scheme is based on the lane index. For example, if the lane index is 0, there is no rotation of that lane. If the lane index is 1, then the bits in the lane are each shifted by 1. If the lane index is 2, then the bits in the lane are each shifted by 2, and so on. Further, a lane index and a frame index is prepended to each lane. The lane index is 12 bits and the frame index is 20 bits.

Once the lanes are shifted, an inner encoding scheme is applied to each lane to encode the binary data as a polynucleotide sequence. The bit to encode from the data (e.g., 144 bits, plus the lane and frame index) are combined with an 8 bit history and a 4 least significant bit (LSB) index. The bits are encoded using a look up table. The bits are encoded at a rate of 1 bit per base. The length of each of the sequences generated is therefore 188 bases in length. A base candidate is generated using the look up table. A base repetition check is further performed to avoid the same bases encoded next to one another. For example, if two bases (e.g., “AA”) are next to one another, then one of the bases is updated (e.g., “AT”). The bit history is then updated and the lane and/or the frame index is incremented. The new bit history is added to the subsequent bit to encode.

GC filtering is subsequently performed on the bases of the oligo nucleotide sequence. About 5% to about 10% of the oligonucleotides are removed during GC filtering. The base content in the final pool is oligonucleotides is about 45% to about 55% GC content.

The final oligonucleotide pool is then synthesized and stored.

### Example 2-Decoding Binary Data from Oligonucleotides Using Mixed Greedy-Maximum Likelihood (ML) Algorithm

Oligonucleotides that are stored on an array according to the general methods of Example 1 are sequenced through pair-end sequencing. The sequenced oligonucleotides are partially decoded to recover a lane index and/or a frame index. The oligonucleotides are clustered according to their lane index.

The clustered oligonucleotides are then aligned. During alignment, the consensus of the oligonucleotides are analyzed using an alignment algorithm. A first position of each of oligonucleotide sequences are initialized to 0, and the consensus of a next two or three bases are analyzed between about 5 oligonucleotide sequence reads. For each oligonucleotides sequence, a decision is made whether the next two or three bases is correct, or whether there is a deletion, an insertion, or a substitution. The position, given the decision, is incremented for each read. These steps are repeated until the end of the read is reached.

An inner codec comprising a decoding scheme is applied to the clustered and aligned oligonucleotide sequences. The decoding scheme comprises a mixed greedy maximum likelihood (ML) algorithm. In this mixed greedy ML algorithm, decoding is performed based on transition probabilities from about 100 most probable states. The decoding transforms the bases in the oligonucleotide sequences into lanes of binary data. Since the bits are encoded at a rate of 1 bit per base in Example 1, each bases is decoded as a bit in the greedy ML algorithm.

Performance of the decoding scheme is improved by knowing where the oligonucleotide sequence ends. The oligonucleotide lengths are determined through pair-end sequencing. Each sequence is a 188-mer oligonucleotides. A drift term is introduced to the greedy ML algorithm, which is an integer associated with the total number of insertions and deletions. Each insertion represents a +1 value and each deletion represents a −1 value. For example, if there are no insertions and 2 deletions, the total drift will be −2. In such an example, the greedy ML algorithm discards all end decoding states other than 186-mer sequences as being invalid. Therefore, the drift term allows the mixed greedy ML algorithm to know which end decoding states are valid, and further improves the performance.

The output from the mixed greedy ML algorithm are lanes of binary data. The lanes include 144 bits of data, 12 bits encoding the lane index, and 20 bits encoding the frame index.

Once the lanes of binary data are obtained, the lanes are arranged into frames based on the lane index and the frame index. The lanes are deshuffled and grouped according to the frame index. A total of 100,000 frames are obtained with each frame containing 4095 lanes.

An outer codec with an error correction scheme is applied to each frame. An outer Reed-Solomon GF (212) code is applied to each frame, which decreases the size of the frame and generates 2500 lanes per frame.

The frames are then merged to generate an output comprising the binary data. Each frame contains a 45 KB of data, and the total recovered binary data is a 4.5 GB data stream.

### Example 3-Encoding and Decoding Binary Data to and from Oligonucleotides Using Codebooks

An LDPC outer codec is applied to data comprising 20,000 bits using the procedure generally described herein. The output from the LDPC outer codec comprises 35,000 bits.

An inner codec using 4 different codebooks of 8 base symbols (per 8 bits) is applied to the output from the outer codec. The codebooks are created to best maximize edit distance between symbols. All possible symbols are first generated from all combinations of bases, and then filtered by maximum repeats and GC content. An initial symbol is selected, followed by a next symbol with maximum edit distance from the initial symbol. Each subsequent symbol is selected based on the previously symbols. Each subsequent symbol is minimized with respect to the maximum average edit distance to all previously selected symbols. This process is repeated until the codebook is full.

The next codebook is generated by starting from a different symbol and minimizing common symbols from the previous codebook. During inner encoding, when encoding i^{th} 8 bit word, the (i modulo 4) codebook is used to find the assigned 8 bases symbol.

During decoding, for each symbol, a likelihood probability for each possible received bases (with a set limit of mutations (or substitutions), deletions and insertions) is pre-computed. The likelihood probability for each possible base is also saved in a file so it can be loaded later as this pre-computation can be expensive. A decoding scheme comprising maximum likelihood (ML) is then performed to recover bits. An outer LDPC code is subsequently applied to recover the data.

### Example 4-Synthesis Optimized Codec for Storing Binary Data

A specific synthesis order is selected to allow for specific base transitions. For example, the synthesis order is A, G, C, T. This can be used to generate an inner codec comprising a codebook. The resulting codebook according to the synthesis order includes the following codewords: [A, G, C, T, AG, AC, AT, GC, GT, AGC, ACT, AGCT]. These 12 codewords can be synthesized with 4 cycles.

Binary data is processed by dividing the data, applying an outer codec, and shuffling the data according to the general methods of Example 1. The inner codec comprising the codebook is applied to the binary data to encode the binary data as a plurality of polynucleotide sequences. Specifically the binary data is mapped onto a plurality of polynucleotide sequences based on the codebook. The mapping can be further optimized based on edit distance, base repeats, or both.

The codecs are applied such that about 50% of the polynucleotide sequences encode for redundancy (e.g., 2× redundancy). Therefore, 6 values of the binary data are mapped to the 12 codewords. These 6 values are equivalent to log 2 (12)=3.58 bits of information. Therefore, 3.58/2=1.79 bits of information are encoded per codeword. In this example, the payload in each of the plurality of polynucleotide sequences is about 100 bits. This requires about 100 bits/1.79 bits per codeword=55.8 codewords. Therefore, the number of synthesis cycles required using the optimized codec is about 55.8 codewords×4 cycles per codeword=224 cycles of synthesis. Without the codec, the number of synthesis cycles required is about 400 cycles (assuming 4 cycles per addition of a base). The implementation of the inner codec allows for at least about half of the features on a surface for polynucleotide synthesis to be deblocked during each synthesis cycle.

### Example 5-Synthesis Optimized Codec with Codebooks

A specific synthesis order is selected to allow for specific base transitions for each layer. A layer comprises an extension of each polynucleotide by at least one base using a device described herein. For example, the synthesis order at each given consecutive layer may comprise repeats of (1) [A, G, C, T], (2) [C, A, T, G], and (3) [T, G, A, C]. This synthesis order can be used to generate an inner codec comprising three codebooks. The first resulting codebook according to the synthesis order includes the following codewords: (1) [A, G, C, T, AG, AC, AT, GC, GT, AGC, ACT, AGCT]. The second codebook includes the following codewords: (2) [C, A, T, G, CA, CT, CG, AT, AG, CAT, CTG, CATG]. The third codebook includes the following codewords: (3) [T, G, A, C, TG, TA, TC, TGA, TAC, TGAC]. Each of these 12 codewords can be synthesized with 4 cycles.

Binary data is processed by dividing the data, applying an outer codec, and shuffling the data according to the general methods of Example 1. The inner codec comprising the codebook is applied to the binary data to encode the binary data as a plurality of polynucleotide sequences. Specifically the binary data is mapped onto a plurality of polynucleotide sequences based on the codebook. The mapping can be further optimized based on edit distance, base repeats, or both.

The codecs are applied such that about 50% of the polynucleotide sequences encode for redundancy (e.g., 2× redundancy). Therefore, 6 values of the binary data are mapped to the 12 codewords. These 6 values are equivalent to log 2 (12)=3.58 bits of information. Therefore, 3.58/2=1.79 bits of information are encoded per codeword. In this example, the payload in each of the plurality of polynucleotide sequences is about 100 bits. This requires about 100 bits/1.79 bits per codeword=55.8 codewords. Therefore, the number of synthesis cycles required using the optimized codec is about 55.8 codewords×4 cycles per codeword=224 cycles of synthesis. Without the codec, the number of synthesis cycles required is about 400 cycles (assuming 4 cycles per addition of a base). The implementation of the inner codec allows for at least about half of the features on a surface for polynucleotide synthesis to be deblocked during each synthesis cycle.

### Example 6-Storage of Digital Information in DNA

Digital information is stored in DNA using a system comprising one or more processing units; a memory in communication with the one or more processing units, and instructions stored in the memory. The instructions are executed on one or more processing units to store the digital information in DNA.

The digital information comprises 1000 objects that are each about 1 MB. The objects include files with text, audio and/or visual information. The 1000 objects are split into 2 pools, where each pool includes 500 objects that are collectively about 500 MB.

In each of the two pools, a pool descriptor, one or more pool items, and an end pool descriptor is generated. Additionally, a first one or more hashes of the data payload of each of the pool items and a second one or more hashes of each of the one or more objects are determined.

The 500 objects in each pool are stored as data payloads in the one or more pool items. Each data payload in a pool item is about 200 to about 400 bits, and about 2×10⁴ to about 4×10⁴ pool items are generated for each object. A hash of each of the data payloads is generated using a hashing module using SHA-256, and is 256 bits. The hash is appended to each pool item.

The pool descriptor includes a version, a pool ID, and a list of pool item descriptors. The version is saved as a first version of the pool (e.g., “001”). The pool ID is a UUID that is a 128-bit random label specific to the pool. The list of pool items descriptors can include a path of an object, a size of an object, a range of the pool item within an object, and an offset of the pool item in a pool. The path of the object includes where an object is located within a plurality of pools (e.g.,/home/pool1). The range of the pool item within an object includes the range of bits of the object in each pool item (e.g., first 0 to 200 bits in pool item 1). The offset of the pool item in a pool includes the payload location of the first byte of each of the one or more pool items in the payload of a pool. For example, the offset of the first pool item in pool 1 is 0 bytes. The range of the first pool item is the first 50 bytes and the offset of the next pool item will be 50 bytes.

The end pool descriptor includes a list of object descriptors. The end pool descriptor includes a path of an object, as well as a hash of an object. The hash of the object is generated using SHA-256, and the hash of the 1 MB object is 256 bits.

An encoding scheme is applied to encode the 2 pools as 2 libraries comprising a plurality of polynucleotides. Each of the pool descriptor, one or more pool items, and end pool descriptor in a pool are encoded as a polynucleotide in the plurality of polynucleotides.

The bits to encode from the pool descriptor, one or more pool items, or end pool descriptor are combined with an 8 bit history and a 4 least significant bit (LSB) index. The bits are encoded using a look up table. The bits are encoded at a rate of 1 to 2 bits per base. The length of each of the sequences generated is about 200 to about 500 bases in length. A base candidate is generated using the look up table. A base repetition check is further performed to avoid the same bases encoded next to one another. For example, if two bases (e.g., “AA”) are next to one another, then one of the bases is updated (e.g., “AT”). The bit history is then updated and the lane and/or the frame index is incremented. The new bit history is added to the subsequent bit to encode.

GC filtering is subsequently performed on the bases of the oligonucleotide sequence. About 5% to about 10% of the oligonucleotides are removed during GC filtering. The base content in the final pool is oligonucleotides is about 45% to about 55% GC content.

The final oligonucleotide pool is then synthesized and stored.

### Example 7-Encoding and Decoding Digital Information in DNA

Two 1 TB objects are stored in 1000 pools, where each pool has a 2 GB maximum payload size. Both pool layouts are first calculated and verified by splitting each object in 2 GB pool items, and ranges and offsets of the pool items are assigned. Each object is processed in 2 GB segments through the low-level codec. At the same time, the hash of each segment and each object is simultaneously calculated and appended to the pool end descriptors.

During decoding, the low-level codec streams out each segment. Because the pool's identity is not necessarily known, the segments are streamed to a destination file representing each object while keeping track of the sections of the object already decoded. When the high-level codec detects that all segments of an object has been decoded, the overall object hash is compared to the stored hash to confirm decoding completeness. Then the object can be flagged as completed and ready for the end user.

### Example 8-Storage and Retrieval of Images in DNA

Images were collected and organized into a directory. The total size of the collected images was around 50 Megabytes. The encoding software with a low level codec (e.g., encoding scheme following the general procedure of Example 1 and as generally illustrated in FIG. 1 and FIGS. 3-6) and a high level codec (e.g., encoding scheme following the general procedure of Example 6 and as generally illustrated in FIG. 12 and FIGS. 14-15) was executed, targeting 198-mer oligo length, including 22-mer forward primer and 18-mer reverse primer. The oligos were synthesized using an inkjet based synthesis method on a chip comprising a solid support. The chip included more than 1.6 million unique sites for synthesis. Therefore, the amount of data per chip was about 10 Megabytes for the selected codec parameters. The codec software automatically divided the data into 5 chips and generated up to 1.6 million oligos per chip to be synthesized.

Five runs on inkjet based synthesizers were executed using standard protocols and resulted in 5 oligo pools. Each pool was sequenced on Illumina sequencers, and the resulting reads were randomly down-selected to get an average of 5× sequencing coverage. The decoding software with a low level codec (e.g., decoding scheme following the general procedure of Example 2 or as generally illustrated in FIG. 2 and FIGS. 8-9) and a high level codec (e.g., decoding scheme following the general procedure of Example 7 or as generally illustrated in FIG. 13) was ran on all 5 sequencing runs, automatically decoding each pool and restitching the data into all the images. All images were successfully recovered with the original directory structure. The codec used multiple levels of SHA256 hashing to verify the recovered data and data structure is 100% accurate.

### Example 9-Storage and Retrieval of PDFs in DNA

Five PDFs of scientific publications were provided, having a total size of about 1 Megabyte. The encoding software with a low level codec (e.g., encoding scheme following the general procedure of Example 1 and as generally illustrated in FIG. 1 and FIGS. 3-6) and a high level codec (e.g., encoding scheme following the general procedure of Example 6 and as generally illustrated in FIG. 12 and FIGS. 14-15) was executed on the provided files as is, targeting 198-mer oligo length, including 22-mer forward primer and 18-mer reverse primer. The oligos were synthesized using an inkjet based synthesis method on a chip comprising a solid support, resulting in one pool of around 100000 oligos.

The pool was amplified, sequenced and decoded (with a low level codec (e.g., decoding scheme following the general procedure of Example 2 or as generally illustrated in FIG. 2 and FIGS. 8-9) and a high level codec (e.g., decoding scheme following the general procedure of Example 7 or as generally illustrated in FIG. 13) to perform a full quality control (QC). The pool was split into multiple copies and stored in individual capsules. The capsules were sent to user with sequencing instructions and a dockerized decoder. The capsules were opened by the user, sequencing was performed, and the decoder was run on their computer. The original PDF data was successfully recovered, showing the reading process could be done on user's computers.

### Example 10-Storage and Retrieval of Payloads in DNA

A 1 Gigabyte payload was built using various downloaded files randomly selected to create representative content, and stored in a directory. The encoding software with a low level codec (e.g., encoding scheme following the general procedure of Example 1 and as generally illustrated in FIG. 1 and FIGS. 3-6) and a high level codec (e.g., encoding scheme following the general procedure of Example 6 and as generally illustrated in FIG. 12 and FIGS. 14-15) was executed on the directory, resulting in roughly 100 million oligos. A simulation software was executed to simulate synthesis, storage and sequencing of this pool using 2% deletion rate, 1% mutation rate and 0.5% insertion rate, and 5× sequencing coverage, resulting in 1 billion reads.

The decoding software with a low level codec (e.g., decoding scheme following the general procedure of Example 2 or as generally illustrated in FIG. 2 and FIGS. 8-9) and a high level codec (e.g., decoding scheme following the general procedure of Example 7 or as generally illustrated in FIG. 13) was executed and successfully recovered the original 1 gigabyte payload. The process took 2 hours on an 8-core Intel® 9i running Ubuntu Linux.

### Example 11-Storage and Retrieval of Payloads with Errors

A 1 Megabyte payload was built using a random generator. The encoding software with a low level codec (e.g., encoding scheme following the general procedure of Example 1 and as illustrated in FIG. 1 and FIGS. 3-6) was executed repeatedly on the resulting payload with varying parameters of redundancy and codeword tables. A simulation software was executed repeatedly to simulate synthesis, storage and sequencing, for parameters ranging from no errors, up to 6% deletion rate, 3% mutation rate and 1% insertion rate, as well as from no oligo loss to 20% oligo loss.

The decoding software (e.g., decoding scheme following the general procedure of Example 2 or as generally illustrated in FIG. 2 and FIGS. 8-9) was executed on each simulation results to verify full payload recovery using different decoding strategies, from pure inner greedy decoder (e.g., FIG. 8) to pure maximum likelihood decoder (e.g., FIG. 9). The greedy decoder started failing at 3% deletion, 2% mutation and 0.5% insertion rates. The maximum likelihood decoder worked until 6% deletion, 3% mutation and 1% insertion rates, which represented a pretty low quality synthesis and sequencing quality. For reference, the general synthesis methods described herein, for example, those used in Examples 8 and 9, have results better than 0.1% deletion, 0.1% mutation and 0.05% insertion rates.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. A method for encoding data, the method comprising:
(a) splitting data into a plurality of frames, wherein each frame in the plurality of frames comprises a frame index;
(b) applying an outer codec to each frame in the plurality of frames, wherein the outer codec comprises an error correction scheme;
(c) dividing each frame into a plurality of lanes, wherein each lane in the plurality of lanes comprises a lane index;
(d) shuffling each lane based at least in part on the lane index; and
(e) applying an inner codec to encode each lane in a corresponding polynucleotide sequence, thereby generating a plurality of corresponding polynucleotide sequences.

2. The method of claim 1, wherein the data comprises binary data, including a byte stream or a byte array.

3. The method of claim 1, wherein the shuffling in (d) comprises a rotation scheme within each lane, comprises a pseudorandom process within each lane, and/or provides resistance against errors.

4. (canceled)

5. (canceled)

6. The method of claim 3, wherein the shuffling in (d) provides resistance against errors, and the errors are nucleotide synthesis errors or sequencing errors.

7. The method of claim 6, wherein the errors comprise a deletion, an insertion, or a substitution.

8. The method of claim 1, wherein the error correction scheme comprises a Reed-Solomon (RS) code, a low-density parity-check (LDPC) code, a Turbo-code, a polar code, or any combination thereof.

9. (canceled)

10. The method of claim 1, wherein:
the plurality of frames comprises about 100 to about 10,000 frames;
each frame comprises less than or equal to about 5000 lanes;
each lane comprises about 100 to about 300 bits;
each frame index comprises about 16 to about 20 bits;
the lane index comprises about 12 bits or about 16 bits; and/or
each corresponding polynucleotide sequence of the plurality of corresponding polynucleotide sequences has a length of about 100 to about 300 bases.

11-15. (canceled)

16. The method of claim 1, wherein the frame index and/or the lane index are prepended to each lane prior to (d).

17. The method of claim 1, wherein applying the inner codec comprises adding redundancy across the plurality of corresponding polynucleotide sequences, wherein the redundancy is about 5% to about 10%.

18-20. (canceled)

21. The method of claim 1, wherein applying the inner codec comprises:
(a) combining symbols from a lane, a symbol history, and a symbol position; and
(b) generating a base candidate using a lookup table, a hash, or both.

22. (canceled)

23. The method of claim 21, further comprising performing a base repetition check, updating the symbol history, incrementing the lane index, incrementing the frame index, or any combination thereof.

24-28. (canceled)

29. The method of claim 1, wherein each lane comprises a plurality of symbols, and applying the inner codec comprises:
(a) generating a base candidate for each symbol within a lane using a lookup table; and
(b) selecting a next lookup table based at least in part on the previously encoded symbol.

30-54. (canceled)

55. A method for encoding data, the method comprising:
(a) generating an inner codec comprising a codebook, wherein the codebook is optimized for one or more constraints; and
(b) applying the inner codec to encode data as a plurality of polynucleotide sequences.

56. The method of claim 55, wherein the one or more constraints are related to nucleic acid synthesis, post-processing, storage, sequencing, or any combination thereof.

57-65. (canceled)

66. The method of claim 55, further comprising synthesizing a plurality of polynucleotides comprising the plurality of polynucleotide sequences.

67. The method of claim 55, wherein the codebook comprises codewords that are generated based at least in part on a base order.

68. The method of claim 67, wherein the base order comprises predetermined base transitions.

69. The method of claim 55, wherein the inner codec comprises two or more codebooks, and
wherein each of the two or more codebooks encodes a layer during synthesis of the plurality of polynucleotides.

70. (canceled)

71. The method of claim 69, wherein the layer comprises extension of each polynucleotide of the plurality of polynucleotides by at least one base.

72-86. (canceled)

87. The method of claim 66, wherein (c) comprises synthesizing the plurality of polynucleotides on a solid support.

88-268. (canceled)
